(19) United States
(12) Patent Application Publication
(10) Pub. No.: US 2006/0276485 A1
(43) Pub. Date: Dec. 7, 2006

(54) Substituted piperidine and piperazine derivatives as melanocortin-4 receptor modulators

(76) Inventors: Michael Soeberdt, Rheinfelden (DE); Philip Weyermann, Sissach (CH); Andreas Von Sprecher, Oberwil (CH)

Correspondence Address: MICHAEL BEST & FRIEDRICH, LLP, 100 E WISCONSIN AVENUE, MILWAUKEE, WI, 53202, US

(21) Appl. No.: 10/550,222

(22) PCT Filed: Mar. 19, 2004
(86) PCT No.: PCT/EP04/02908

§ 371 Date: Jun. 5, 2006

(30) Foreign Application Priority Data
Mar. 20, 2003 (EP) 03006253.3

(51) Int. Cl. A61K 31/496; A61K 31/4709; A61K 31/454; C07D 405/14; C07D 403/14

(52) U.S. Cl. 514/253.11; 514/254.01; 514/320; 514/312; 544/364; 544/373; 546/153; 546/207; 514/253.07; 544/363

(57) ABSTRACT

The present invention relates to novel substituted piperidine and piperazine derivatives as melanocortin-4 receptor (MC-4R) modulators. MC-4R agonists of the invention can be used for the treatment of disorders and diseases such as obesity, diabetes, and sexual dysfunction, whereas the MC-4R antagonists are useful for the treatment of disorders and diseases such as cancer cachexia, muscle wasting, anorexia, anxiety and depression. All diseases and disorders where the regulation of the MC-4R is involved can be treated with the compounds of the invention.

### FIELD OF THE INVENTION

The present invention relates to novel substituted piperidine and piperazine derivatives as melanocortin-4 receptor modulators. Depending on the structure and the stereochemistry the compounds of the invention are either selective agonists or selective antagonists of the human melanocortin-4 receptor (MC-4R). The agonists can be used for the treatment of disorders and diseases such as obesity, diabetes and sexual dysfunction, whereas the antagonists are useful for the treatment of disorders and diseases such as cancer cachexia, muscle wasting, anorexia, anxiety and depression. Generally all diseases and disorders where the regulation of the MC-4R is involved can be treated with the compounds of the invention.

### BACKGROUND OF THE INVENTION

Melanocortins (MCs) stem from pro-opiomelanocortin (POMC) via proteolytic cleavage. These peptides, adrenocorticotropic hormone (ACTH), α-melanocyte-stimulating hormone (α-MSH), β-MSH and γ-MSH, range in size from 12 to 39 amino acids. The most important endogenous agonist for central MC-4R activation appears to be the tridecapeptide α-MSH. Among MCs, it was reported that α-MSH acts as a neurotransmitter or neuromodulator in the brain. MC peptides, particularly α-MSH, have a wide range of effects on biological functions including feeding behavior, pigmentation, and exocrine function. The biological effects of α-MSH are mediated by a subfamily of 7-transmembrane G-protein-coupled receptors, termed melanocortin receptors (MC-Rs). Activation of any of these MC-Rs results in stimulation of CAMP formation.

To date, five distinct types of receptor subtype for MC (MC-1R to MC-5R) have been identified, and these are expressed in different tissues.

MC-1R was first found in melanocytes. Naturally occurring inactive variants of MC-1R in animals were shown to lead to alterations in pigmentation and a subsequent lighter coat color by controlling the conversion of phaeomelanin to eumelanin through the control of tyrosinase. From these, and other studies, it is evident that MC-1R is an important regulator of melanin production and coat color in animals and skin color in humans.

The MC-2R is expressed in the adrenal gland representing the ACTH receptor. The MC-2R is not a receptor for α-MSH but is the receptor for the adrenocorticotropic hormone I (ACTH I).

The MC-3R is expressed in the brain (predominately located in the hypothalamus) and peripheral tissues like gut and placenta, and knock-out studies have revealed that the MC-3R may be responsible for alterations in feeding behavior, body weight and thermogenesis.

The MC-4R is primarily expressed in the brain. Overwhelming data support the role of MC-4R in energy homeostasis. Genetic knock-outs and pharmacologic manipulation of MC-4R in animals have shown that agonizing the MC-4R causes weight loss and antagonizing the MC-4R produces weight gain. (A. Kask, et al., “Selective antagonist for the melanocortin-4 receptor (HS014) increases food intake in free-feeding rats,” Biochem. Biophys. Res. Commun., 245:90-93 (1998)).

MC-5R is ubiquitously expressed in many peripheral tissues including white fat, placenta and a low level of expression is also observed in the brain. However its expression is greatest in exocrine glands. Genetic knock-out of this receptor in mice results in altered regulation of exocrine gland function, leading to changes in water repulsion and thermoregulation. MC-5R knockout mice also reveal reduced sebaceous gland lipid production (Chen et al., Cell, 91: 789-798 (1997)).

Attention has been focused on the study of MC-3R and MC-4R modulators and their use in treating body weight disorders, such as obesity and anorexia. However, evidence has shown that the MC peptides have potent physiological effects besides their role in regulating pigmentation, feeding behavior and exocrine function. In particular, α-MSH recently has been shown to induce a potent anti-inflammatory effect in both acute and chronic models of inflammation including inflammatory bowel-disease, renal ischemia/reperfusion injury and endotoxin-induced hepatitis. Administration of α-MSH in these models results in substantial reduction of inflammation-mediated tissue damage, a significant decrease in leukocyte infiltration, and a dramatic reduction in elevated levels of cytokines and other mediators to near baseline levels. Recent studies have demonstrated that the anti-inflammatory actions of α-MSH are mediated by MC-1R. The mechanism by which agonism of MC-1R results in an anti-inflammatory response is likely through inhibition of the pro-inflammatory transcription activator, NF-κB. NF-κB is a pivotal component of the pro-inflammatory cascade, and its activation is a central event in initiating many inflammatory diseases. Additionally, anti-inflammatory actions of α-MSH may be in part mediated by agonism of MC-3R and/or MC-5R.

A specific single MC-R that may be targeted for the control of obesity has not yet been identified, although evidence has been presented that MC-4R signaling is important in mediating feeding behavior (S. Q. Giraudo et al., “Feeding effects of hypothalamic injection of melanocortin-4 receptor ligands”, Brain Research, 80: 302-306 (1998)). Further evidence for the involvement of MC-Rs in obesity includes: a) the agouti (A^{vy}) mouse which ectopically expresses an antagonist of the MC-1R, MC-3R and MC-4R is obese, indicating that blocking the action of these three MC-Rs can lead to hyperphagia and metabolic disorders; 2) MC-4R knockout mice (D. Huszar et al., Cell, 88: 131-141 (1997)) recapitulate the phenotype of the agouti mouse and these mice are obese; 3) the cyclic heptapeptide melanotanin 11 (MT-II) (a non-selective MC-1R, -3R, 4R and -5R agonist) injected intracerebroventricularly (ICV) in rodents, reduces food intake in several animal feeding models (NPY, ob/ob, agouti, fasted), while ICV injected SHU-9119 (MC-3R and -4R antagonist; MC-1R, and -5R agonist) reverses this effect and can induce hyperphagia; 4) chronic intraperitoneal treatment of Zucker fatty rats with an α-NDP-MSH derivative (HP-228) has been reported to activate MC1-R, -3R, -4R and -5R and to attenuate food intake and body weight gain over a 12 week period (I. Corcos et al., “HP-228 is a potent agonist of melanocortin receptor-4 and significantly attenuates obesity and diabetes in Zucker fatty rats”, Society for Neuroscience Abstracts, 23: 673 (1997)).

MC-4R appears to play a role in other physiological functions as well, namely controlling grooming behavior, erection and blood pressure. Erectile dysfunction denotes the medical condition of inability to achieve penile erection sufficient for successful intercourse. The term “impotence” is often employed to describe this prevalent condition. Synthetic melanocortin receptor agonists have been found to initiate erections in men with psychogenic erectile dysfunction (H. Wessells et al., “Synthetic Melanotropic Peptide Initiates Erections in Men With Psychogenic Erectile Dysfunction: Double-Blind, Placebo Controlled Crossover Study”, J. Urol., 160: 389-393, 1998). Activation of melanocortin receptors of the brain appears to cause normal stimulation of sexual arousal. Evidence for the involvement of MC-R in male and/or female sexual dysfunction is detailed in WO/0074679.

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II diabetes” or “Non-insulin Dependent Diabetes Mellitus” (NIDDM) is the form of diabetes, which is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. When these cells become desensitized to insulin, the body tries to compensate by producing abnormally high levels of insulin and hyperinsulemia results. Hyperinsulemia is associated with hypertension and elevated body weight. Since insulin is involved in promoting the cellular uptake of glucose, amino acids and triglycerides from the blood by insulin sensitive cells, insulin insensitivity can result in elevated levels of triglycerides and LDL which are risk factors in cardiovascular diseases. The constellation of symptoms which includes hyperinsulemia combined with hypertension, elevated body weight, elevated triglycerides and elevated LDL is known as Syndrome X. MC-4R agonists might be useful in the treatment of NIDDM and Syndrome X.

Among MC receptor subtypes, the MC4 receptor is also of interest in terms of the relationship to stress and the regulation of emotional behavior, as based on the following findings. Stress initiates a complex cascade of responses that include endocrine, biochemical and behavioral events. Many of these responses are initiated by release of corticotropin-releasing factor (CRF), (Owen M J and Nemeroff C B (1991). Physiology and pharmacology of corticotrophin releasing factor. Pharmacol Rev 43: 425-473). In addition to activation of the brain CRF system, there are several lines of evidence that melanocortins (MCs), which stem from proopiomelanocortin by enzymatic processing, mediate important behavioral and biochemical responses to stress and, consequently, stress-induced disorders like anxiety and depression (Anxiolytic-Like and Antidepressant-Like Activities of MCL0129 (1-[(S)-2-(4-Fluorophenyl)-2-(4-isopropylpiperadin-1-yl)ethyl]-4-[4-(2-methoxynaphthalen-1-yl)butyl]piperazine), a Novel and Potent Nonpeptide Antagonist of the Melanocortin-4 Receptor; Shigeyuki Chaki et al, J. Pharm. Exp. Ther. (2003) 304(2), 818-26).

Chronic diseases such as malignant tumors or infections are frequently associated with cachexia resulting from a combination of a decrease in appetite and a loss of lean body mass. Extensive loss of lean body mass is often triggered by an inflammatory process and is usually associated with increased plasma levels of cytokines (e.g. TNF-α), which increase the production of α-MSH in the brain. Activation of MC4 receptors in the hypothalamus by α-MSH reduces appetite and increases energy expenditure. Experimental evidence in tumor bearing mice suggests that cachexia can be prevented or reversed by genetic MC4 receptor knockout or MC4 receptor blockade. The increased body weight in the treated mice is attributable to a larger amount of lean body mass, which mainly consists of skeletal muscle (Marks D. L. et al. Role of the central melanocortin system in cachexia. Cancer Res. (2001) 61: 1432-1438).

WO03009847A1 describes phenylpiperidinyl-phenylalanine derivatives and WO03009850A1 describes phenylpiperazinyl-phenylalanine derivatives for the treatment of obesity. Most of the compounds in both patents contain a N-(2-piperidin-4-yl-phenyl)-alkyl, benzyl or aryl sulfonamide group and N-(2-piperazin 4-yl-phenyl)-alkyl, benzyl or aryl sulfonamide group, respectively. In WO030009847A1 four out of 429 described examples bear the 1-(2-piperidin-4-yl-benzyl)-pyrrolidin-2-one-4-yl group and one example bears the 1-(2-piperidin-4-yl-benzyl)-piperidin-2-one-4-yl group, in WO030009850A1 four out of 456 described examples bear the 1-(2-piperazin-1-yl-benzyl)-pyrrolidin-2-one-4-yl group and one example bears the 1-(2-piperazin-1-yl-benzyl)-piperidin-2-one-4-yl group. However, neither the synthesis of the intermediates 1-(2-piperidin-4-yl-benzyl)-pyrrolidin-2-one and 1-(2-piperidin-4-yl-benzyl)-piperidin-2-one nor the synthesis of the intermediates 1-(2-piperazin-1-yl-benzyl)-pyrrolidin-2-one and 1-(2-piperazin-1-yl-benzyl)-piperidin-2-one nor the synthesis of the corresponding final products are described. All of the ten compounds have in common the p-chlorophenylalanine moiety which is acylated with unsubstituted and substituted azetidine-3-carboxylic acids. Other amino acids were not used to acylate the p-chlorophenylalanine. Biological data (e.g. binding IC₅₀ or functional activity) are not provided. WO02070511A1 describes phenylpiperazinyl-phenylalanine amides, phenylpiperidinyl-phenylalanine amides and cyclohexyl-phenylalanine amides as modulators of melanocortin receptors 1 and 4. The phenylalanine amino group is in the most cases acylated with a second amino acid. For amino acids with a basic side chain the amino group can be acylated. Biological data for the compounds are not given

In view of the unresolved deficiencies in treatment of various diseases and disorders as discussed above, it is an object of the present invention to provide novel substituted piperidine and piperazine derivatives with improved ability to cross the blood brain barrier, which are useful as melanocortin-4 receptor modulators to treat cancer cachexia, muscle wasting, anorexia, anxiety, depression, obesity, diabetes, sexual dysfunction, and other diseases with MC-4R involvement.

### SUMMARY OF THE INVENTION

The present invention relates to novel substituted piperidine and piperazine derivatives of the following general structural formula.

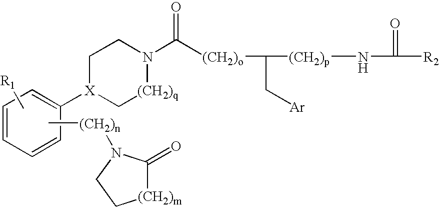

These piperidine and piperazine derivatives are effective as melanocortin receptor modulators and are particularly effective as selective melanocortin-4 receptor (MC-4R) modulators. They are therefore useful for the treatment of disorders where the activation or inactivation of the MC-4R are involved. Agonists can be used for the treatment of disorders and diseases such as obesity, diabetes, and sexual dysfunction, whereas the antagonists are useful for the treatment of disorders and diseases such as cancer cachexia, muscle wasting, anorexia, anxiety and depression.

The present invention also relates to pharmaceutical compositions comprising the compounds of the present invention and a pharmaceutically acceptable carrier.

### DETAILED DESCRIPTION OF THE INVENTION

The present invention relates to novel substituted piperidine and piperazine derivatives useful as melanocortin receptor modulators, in particular, selective MC-4R agonists and MC-4R antagonists.

The compounds of the present invention are represented by structural formula (I). 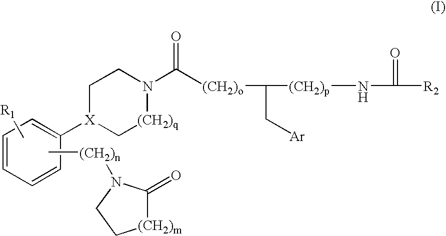
or a pharmaceutically acceptable salt or solvate thereof, wherein

Ar is:
aryl or heteroaryl which may both be substituted or unsubstituted;

R₁ is independently:
hydrogen,
hydroxy,
cyano,
nitro,
halo,
alkyl,
alkoxy or
haloalkyl;

R₂ is:

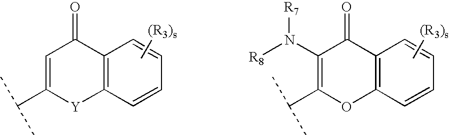

each R₃ is independently:
hydrogen,
halo,
alkyl,
haloalkyl,
hydroxy,
alkoxy,
S-alkyl,
SO₂-alkyl,
O-alkenyl,
S-alkenyl,
NR₇C(O)R₇,
NR₇SO₂R₇,
N(R₇)₂
(D)-cycloalkyl,
(D)-aryl,
(D)-heteroaryl or
(D)-heterocyclyl,
wherein heterocyclyl excludes a heterocyclyl containing a single nitrogen, wherein aryl, heteroaryl, heterocyclyl, alkyl and/or cycloalkyl may be substituted or unsubstituted, and two adjacent R₃ may form a 4- to 7-membered ring;

R₇ and R₈ are each independently:
hydrogen,
alkyl or
cycloalkyl, or
R₇ and R₈ together with the nitrogen to which they are attached form a 5- to 8-membered ring,
wherein alkyl and cycloalkyl are both unsubstituted or substituted;

D is a bond or alkyl;

X is CH or N;

Y is O or NR₇;

n is 1-4;

m is 0-3;

o is 0-2;

p is 0-2;

q is 1 or 2;

s is 0-4.

In preferred embodiments, the variants of formula (I) have the following meanings:

Ar is as defined above, and is preferably aryl, more preferably phenyl or naphthyl. If aryl or heteroaryl are substituted, it is preferably substituted with one to three, more preferably one or two, most preferably one, substituents. The substituents are preferably independently selected from the group consisting of: cyano, nitro, perfluoroalkoxy, halo, alkyl, (D)-cycloalkyl, alkoxy and haloalkyl, more preferably perfluoroalkoxy, halo, alkyl, alkoxy or haloalkyl, even more preferably halo, alkyl, alkoxy and haloalkyl, in particular halo.

Most preferably, Ar is phenyl or naphthyl which both, preferably phenyl, may be substituted with one to three, in particular one, halo, e.g. Cl. The substitution can be in any position, preferably in the 4-position.

R₁ is as defined above, preferably hydrogen, hydroxy, halo, alkyl, alkoxy or haloalkyl, more preferably hydrogen, alkoxy, halo or alkyl, most preferably hydrogen.

R₂ is each of the rings as defined above.

In formula (I), R₂ is most preferably

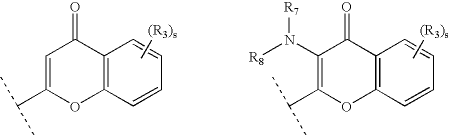

R₃ is as defined above. If aryl, heteroaryl, heterocyclyl, alkyl and/or cycloalkyl are substituted, they are independently preferably substituted with one to three, more preferably one substituent selected from the group consisting of oxo, halo, alkyl, N(R₄)₂, OR₄, SR₄ and CO₂R₄.

R₃ is preferably hydrogen, halo, unsubstituted alkyl, substituted alkyl, haloalkyl, hydroxyl, alkoxy, S-alkyl, SO₂-alkyl, O-alkenyl, S-alkenyl, more preferably hydrogen, iso-propyl, hydroxyl, alkoxy, S-alkyl, and, SO₂-alkyl. In one embodiment R₃ is hydrogen, halo, alkyl, haloalkyl, alkoxy, (D)-cycloalkyl, (D)-aryl, (D)-heteroaryl or (D)-heterocyclyl, wherein heterocyclyl excludes a heterocyclyl containing a single nitrogen, wherein aryl, heteroaryl, heterocyclyl, alkyl and/or cycloalkyl may be substituted or unsubstituted; preferably hydrogen, halo, unsubstituted alkyl, substituted alkyl, haloalkyl, alkoxy, unsubstituted (D)-cycloalkyl or substituted (D)-cycloalkyl; more preferably hydrogen.

R₄ is independently hydrogen, alkyl, C(O)alkyl, SO₂alkyl, SO₂aryl, (D)-aryl or cycloalkyl. Preferably, R₄ is hydrogen or alkyl, more preferably hydrogen.

R₇ and R₈ are each independently as defined above. When R₇ and R₈ form a ring, said ring may contain an additional heteroatom, preferably selected from O, S and NR₄ in the ring. Moreover, if alkyl and cycloalkyl are substituted, they are preferably substituted with one to three, more preferably one or two groups independently selected from R₉ and oxo.

R₇ and R₈ are each independently preferably selected from the group consisting of hydrogen, alkyl and cycloalkyl; or R₇ and R₈ together with the nitrogen to which they are attached form a 5- to 7-membered ring. More preferably R₇ and R₈ are each independently selected from the group consisting of hydrogen and alkyl; or R₇ and R₈ together with the nitrogen to which they are attached form a 5- to 6-membered ring optionally containing an additional oxygen atom.

R₉ is alkyl, (D)-aryl, (D)-cycloalkyl, (D)-heteroaryl, halo, OR₁₀, NHSO₂R₁₀, N(R₁₀)₂, C≡N, CO₂R₇, C(R₁₀)(R₁₀)N(R₁₀)₂, nitro, SO₂N(R₁₀)₂, S(O)ₙR₁₀, CF₃ or OCF₃, and preferably selected from the group consisting of alkyl, OR₁₀, (D)-aryl, (D)-cycloalkyl, (D)-heteroaryl and halo.

R₁₀ is independently hydrogen, alkyl, (D)-aryl or cycloalkyl, preferably hydrogen or alkyl, more preferably alkyl.

D is as defined above, preferably a bond or CH₂, most preferably a bond.

X is as defined above. In one embodiment, X is CH.

Y is as defined above, preferably O. In one embodiment Y is NR₇, more preferably N-alkyl. Alkyl is as defined below, preferably C₁-C₄ alkyl. In one embodiment, Y is N-propyl.

n is as defined above, preferably 1 or 2, more preferably 1.

m is as defined above, preferably 1, 2 or 3, most preferably 1 or 2.

o is as defined above, preferably 0 or 1 most preferably o is 0.

p is as defined above, preferably 0 or 1 most preferably p is 0.

q is as defined above, preferably 1.

s is as defined above, i.e. 0, 1, 2, 3 or 4, preferably 1, 2 or 3, most preferably 1 or 2.

u is 0, 1 or 2.

In the above, any of the preferred definitions for each variant can be combined with the preferred definition of the other variants.

In the above and the following, the employed terms have the meaning as described below:

Aryl is an aromatic mono- or polycyclic moiety with 6 to 20 carbon atoms which is preferably selected from phenyl, biphenyl, naphthyl, tetrahydronaphthyl, fluorenyl, indenyl and phenanthrenyl, more preferably from phenyl and naphthyl.

Heteroaryl is an aromatic moiety having 6 to 20 carbon atoms with at least one heterocycle and is preferably selected from thienyl, benzothienyl, naphthothienyl, furanyl, benzofuranyl, chromenyl, indolyl, isoindolyl, indazolyl, quinolyl, isoquinolyl, phthalazinyl, quinoxalinyl, cinnolinyl and quinazolinyl, more preferably from thienyl, furanyl, benzothienyl, benzofuranyl and indolyl.

Heterocyclyl is a saturated, unsaturated or aromatic ring containing at least one heteroatom selected from O, N and/or S and 1 to 6 carbon atoms and is preferably selected from thienyl, furyl, piperidinyl, pyranyl, pyrrolyl, imidazolyl, pyrazolyl, pyridyl, pyrazinyl, pyrimidinyl, pyridazinyl, isothiazolyl and isoxazyl, more preferably from pyridyl, piperidinyl, imidazolyl and pyrazinyl.

Carbocyclyl is a monocyclic or polycyclic ring system of 3 to 20 carbon atoms which may be saturated, unsaturated or aromatic.

Alkyl is straight chain or branched alkyl having preferably 1 to 8 carbon atoms, such as methyl, ethyl, n-propyl, isopropyl, n-butyl, isobutyl, tert-butyl, n-pentyl, isopentyl, neopentyl, hexyl or heptyl, more preferably 1 to 4 carbon atoms.

Cycloalkyl is an alkyl ring having preferably 3 to 8 carbon atoms, such as cyclopropyl, cyclobutyl, cyclopentyl, cyclohexyl or cycloheptyl, more preferably 3 to 6 carbon atoms.

Alkenyl is straight chain or branched alkenyl having preferably 2 to 8 carbon atoms such as vinyl, allyl, methallyl, buten-2-yl, buten-3-yl, penten-2-yl, penten-3yl, penten-4-yl, 3-methyl-but-3-enyl, 2-methyl-but-3-enyl, 1-methyl-but-3-enyl, hexenyl or heptenyl, more preferably 2 to 4 atoms.

Alkoxy is O-alkyl wherein alkyl is as defined above and has preferably 1 to 4 carbon atoms, more preferably 1 or 3 carbon atoms.

Halo or halogen is a halogen atom preferably selected from F, Cl, Br and I, more preferably from F, Cl and Br.

Haloalkyl is an alkyl moiety as defined above having preferably 1 to 4 carbon atoms, more preferably 1 or 2 carbon atoms, wherein at least one, preferably 1, 2 or 3 hydrogen atoms have been replaced by a halogen atom. Preferred examples are —CF₃—CH₂CF₃ and —CF₂CF₃.

The compounds of structural formula (I) are effective as melanocortin receptor modulators and are particularly effective as selective modulators of MC-4R. They are therefore useful for the treatment and/or prevention of disorders responsive to the activation and inactivation of MC-4R, such as cancer cachexia, muscle wasting, anorexia, anxiety, depression, obesity, diabetes, sexual dysfunction and other diseases with MC-4R involvement.

Optical Isomers13 Diastereomers—Geometric Isomers—Tautomers

The compounds of structural formula (I) contain one or more asymmetric centers and can occur as racemates and racemic mixtures, single enantiomers, diastereomeric mixtures and individual diastereomers. The present invention is meant to comprehend all such isomeric forms of the compounds of structural formula (I).

Some of the compounds described herein may exist as tautomers such as keto-enol tautomers. The individual tautomers, as well as mixtures thereof, are encompassed within the compounds of structural formula (I).

The compounds of structural formula (I) may be separated into their individual diastereoisomers by, for example, fractional crystallization from a suitable solvent, for example, methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration. Alternatively, any stereoisomer of a compound of the general formula (I) may be obtained by stereospecific synthesis using optically pure starting materials or reagents of known absolute configuration.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic, manganous, potassium, sodium, zinc, salts and the like. Particularly preferred are the ammonium, calcium, lithium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, ptoluenesulfonic, trifluoroacetic acid and the like. Particularly preferred are citric, fumaric, hydrobromic, hydrochloric, maleic, phosphoric, sulfuric and tartaric acids.

It will be understood that, as used herein, references to the compounds of formula (I) are meant to also include the pharmaceutically acceptable salts.

Utility

The compounds of formula (I) are melanocortin receptor modulators and, as such, are useful in the treatment, contro or prevention of diseases, disorders or conditions responsive to the activation or inactivation of one or more of the melanocortin receptors including, but not limited to, MC-1R, MC-2R, MC-3R, MC-4R and MC-5R. Such diseases, disorders or conditions include, but are not limited to, cancer cachexia, muscle wasting, anorexia, anxiety, depression, obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction (including impotence, loss of libido and erectile dysfunction), fever, inflammation, immune-modulation, rheumatoid arthritis, skin tanning, acne and other skin disorders, neuroprotective and cognitive and memory enhancement, including the treatment of Alzheimer's disease.

Some compounds encompassed by formula (I) show highly selective affinity for the melanocortin-4 receptor relative to MC-1R, MC-2R, MC-3R and MC-5R, which makes them especially useful in the prevention and treatment of cancer cachexia, muscle wasting, anorexia, anxiety, depression, obesity, as well as male and/or female sexual dysfunction, including erectile dysfunction. “Male sexual dysfunction” includes impotence, loss of libido and erectile dysfunction. “Female sexual dysfunction” can be seen as resulting from multiple components, including dysfunction in desire, sexual arousal, sexual receptivity and orgasm.

Administration and Dose Ranges

Any suitable route of administration may be employed for providing a mammal, especially a human with an effective dosage of a compound of the present invention. For example, oral, rectal, topical, parenteral, ocular, pulmonary, nasal and the like may be employed. Dosage forms include tablets, troches, dispersions, suspensions, solutions, capsules, creams, ointments, aerosols and the like. Preferably the compounds of formula (I) are administered orally or topically.

The effective dosage of active ingredient employed may vary depending on the particular compound employed, the mode of administration, the condition being treated and the severity of the condition being treated. Such dosage may be ascertained readily by a person skilled in the art.

When treating cancer cachexia, muscle wasting or anorexia, generally satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating obesity, in conjunction with diabetes and/or hyperglycemia, or alone, generally satisfactory results are obtained when the compounds of the present invention are administered at a daily dosage of from about 0.001 milligram to about 100 milligrams per kilogram of body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating diabetes mellitus and/or hyperglycemia as well as other diseases or disorders for which the compounds of formula (I) are useful, generally satisfactory results are obtained when the compounds of the present invention are administered at a daily dosage of from about 0.001 milligram to about 100 milligram per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

For the treatment of sexual dysfunction, the compounds of the present invention are given in a dose range of 0.001 milligram to about 100 milligram per kilogram of body weight, preferably as a single dose orally, or as a nasal spray.

Formulation

The compound of formula (I) is preferably formulated into a dosage form prior to administration. Accordingly, the present invention also includes a pharmaceutical. composition comprising a compound of formula (I) and a suitable pharmaceutical carrier.

The present pharmaceutical compositions are prepared by known procedures using well-known and readily available ingredients. In making the compositions of the present invention, the active ingredient (a compound of formula (I)) is usually mixed with a carrier or diluted by a carrier or enclosed within a carrier, which may be in the form of a capsule, sachet, paper or other container. When the carrier serves as a diluent, it may be a solid, semisolid, or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions or sterile packaged powders.

Some examples of suitable carriers, excipients and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

Preparation of Compounds of the Invention

When describing the preparation of the compounds of formula (I), the terms “A moiety”, “B moiety”, and “C moiety” are used below. This moiety concept is illustrated below:

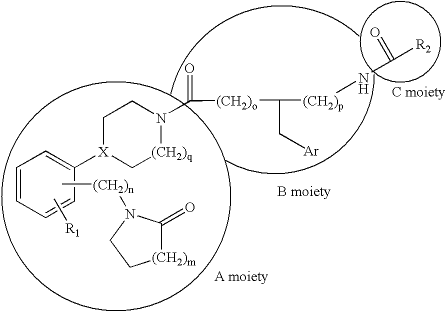

The preparation of the compounds of the present invention may be carried out via sequential or convergent synthetic routes. The skilled artisan will recognize that, in general, the three moieties of a compound of formula (I) are connected via amide bonds. The skilled artisan can, therefore, readily envision numerous routes and methods of connecting the three moieties via standard peptide coupling reaction conditions.

The phrase “standard peptide coupling reaction conditions” means coupling a carboxylic acid with an amine using an acid activating agent such as EDC, dicyclohexylcarbodiimide or benzotriazol-1-yloxytris(dimethylamino)-phosphonium hexafluorophosphate, in an inert solvent such as DCM, in the presence of a catalyst such as HOBt. The uses of protective groups for amine and carboxylic acids to facilitate the desired reaction and minimize undesired reactions are well documented. Conditions required to remove protecting groups which may be present can be found in Greene et al., Protective Groups in Organic Synthesis, John Wiley & Sons, Inc., New York, N.Y. 1991.

Protecting groups like Z, Boc or Fmoc are used extensively in the synthesis, and their removal conditions are well known to those skilled in the art. For example, removal of Z groups can be achieved by catalytic hydrogenation with hydrogen in the presence of a noble metal or its oxide, such as palladium on activated carbon in a protic solvent such as ethanol. In cases where catalytic hydrogenation is contraindicated by the presence of other potentially reactive functionality, removal of Z can also be achieved by treatment with a solution of hydrogen bromide in acetic acid or by treatment with a mixture of TFA and dimethylsulfide. Removal of Boc protecting groups is carried out in a solvent, such as methylene chloride, methanol or ethyl acetate, with a strong acid, such as TFA, HCl or hydrogen chloride gas.

The compounds of formula (I), when existing as a diastereomeric mixture, may be separated into diastereomeric pairs of enantiomers by fractional crystallization from a suitable solvent such as methanol, ethyl acetate or a mixture thereof. The pair of enantiomers, thus obtained, may be separated into individual stereoisomers by conventional means using an optically active acid as a resolving agent. Alternatively, any enantiomer of a compound of formula (I) may be obtained by stereospecific synthesis using optically pure starting materials or reagents of known configuration.

The compounds of formula (I) of the present invention can be prepared according to the procedures of the following schemes and examples using appropriate materials and are further exemplified by the following specific examples. Moreover, by utilizing the procedures described herein, in conjunction with ordinary skills in the art, additional compounds of the present invention can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously. The free amine bases corresponding to the isolated salts can be generated by neutralization with a suitable base, such as aqueous sodium hydrogencarbonate, sodium carbonate, sodium hydroxide or potassium hydroxide, and extraction of the liberated amine free base into an organic solvent, followed by evaporation. The amine free base, isolated in this manner, can be further converted into another pharmaceutically acceptable salt by dissolution in an organic solvent, followed by addition of the appropriate acid and subsequent evaporation, precipitation or crystallization. All temperatures are degrees Celsius. Mass spectra (MS) were measured by electron-spray ion-mass spectroscopy.

In the schemes, preparations and examples below, the various reagent symbols and abbreviations have the following meanings:

BINAP 2,2′-Bis(diphenylphosphino)-1,1′-binaphtyl

Boc t-butoxycarbonyl

Bz₂O₂ dibenzoylperoxide

DCM dichloromethane

DIPEA diisopropylethylamine

DMAP 4-dimethylaminopyridine

DME dimethoxyethane

DMF N,N-dimethylformamide

EDC 1-(3-dimethylaminopropyl)-3-ethylcarbodiimide hydrochloride

Et ethyl

EtOAc ethyl acetate

Fmoc 9-fluorenylmethyl-carbamate

HOAc acetic acid

HOAt 1-hydroxy-7-azabenzotriazole

HOBt 1-hydroxybenzotriazole

h hour(s)

NBS N-bromosuccinimide

NMM N-methylmorpholine

Me methyl

Ms methanesulfonyl

Pd₂(dba)₃ tris(dibenzylideneacetone) dipalladium(0)

Phe phenylalanine

TFA trifluoroacetic acid

TEA triethylamine

Tic 1,2,3,4-tetrahydroisoquinoline-3-carboxylic acid

TMOF trimethylorthoformate

z benzyloxycarbonyl

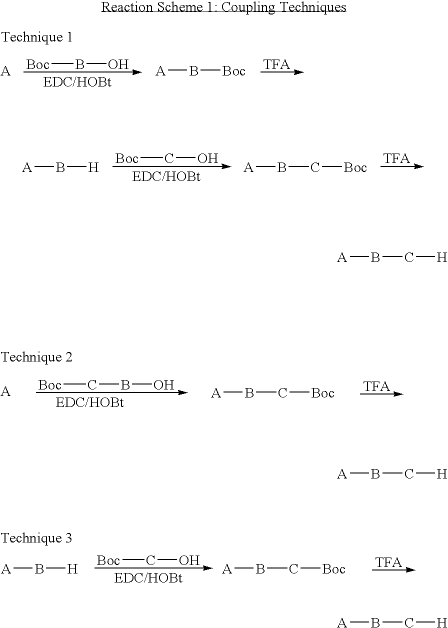

In coupling technique 1, an appropriate “A moiety” (e.g., 1-(2-piperazin-1-yl-benzyl)-pyrrolidin-2-one) is coupled to “B moiety” (e.g., L-Boc-p-Cl-Phe-OH) in the presence of EDC/HOBt followed by Boc deprotection. The coupled AB compound is then coupled to an appropriate “C moiety”, followed by deprotection of Boc group and salt formation. Alternatively, when “C moiety” is not protected with Boc group, the final compound can be obtained without the deprotection step.

In coupling technique 2, an appropriate “AB moiety” is coupled to an appropriate “C moiety” in the presence of EDC/HOBt, followed by deprotection of Boc group and salt formation. Alternatively, when “C moiety” is not protected with Boc group, the final compound can be obtained without the deprotection step.

In coupling technique 3, an appropriate “BC moiety” is coupled to an appropriate “A moiety” in the presence of EDC/HOBt, followed by deprotection of Boc group and salt formation. Alternatively, when “C moiety” is not protected with Boc group, the final compound can be obtained without the deprotection step.

For coupling of A with Boc-B—OH, EDC/HOAt, EDC/HOBt or DCC/HOBt can be used.

Generally, the starting material of Boc-protected piperazine or piperidine (A moiety) can be deprotected in the presence of TFA/CH₂Cl₂, HCl/EtOAc, HCl/dioxane or HCl in MeOH/Et₂O, with or without a cation scavenger, such as dimethyl sulfide (DMS), before being subjected to the coupling procedure. It can be free-based before being subjected to the coupling procedure or, in some cases, used as the salt.

A suitable solvent such as CH₂Cl₂, DMF, THF or a mixture of the above solvents, can be used for the coupling procedure. A suitable base includes triethylamine (TEA), diisopropylethylamine (DIPEA), N-methylmorpholine (NMM), collidine and 2,6-lutidine. A base may not be needed when EDC/HOBt is used.

Generally after the reaction is completed, the reaction mixture can be diluted with an appropriate organic solvent, such as EtOAc, CH₂Cl₂ or Et₂O, which is then washed with aqueous solutions, such as water, HCl, NaHSO₄, bicarbonate, NaH₂PO₄, phosphate buffer (pH 7), brine or any combination thereof. The reaction mixture can be concentrated and then be partitioned between an appropriate organic solvent and an aqueous solution. The reaction mixture can be concentrated and subjected to chromatography without aqueous workup.

Protecting groups such as Boc, Z, Fmoc or CF₃CO, can be deprotected in the presence of H₂/Pd-C, TFA/DCM, HCl/EtOAc, HCl/dioxane, HCl in MeOH/Et₂O, NH₃/MeOH or TBAF with or without a cation scavenger, such as thioanisole, ethane thiol or dimethyl sulfide (DMS). The deprotected amines can be used as the resulting salt or are free-based by dissolving in DCM and washing with aqueous bicarbonate or aqueous NaOH. The deprotected amines can also be free-based by ion exchange chromatography.

Reaction Schemes for Preparation of “A moiety”

The “A moieties” of the present invention, in general, may be prepared from commercially available starting materials via known chemical transformations. The preparation of “A moiety” of the compound of the present invention is illustrated in the reaction scheme below.

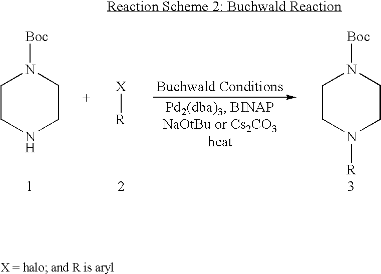

As shown in Reaction Scheme 2, the “A moiety” of the compounds of the present invention can be prepared by coupling halo-substituted aryl 2 (X—R) with 1-Boc-piperazine 1 in the presence of tri(dibenzylideneacetone) dipalladium (Pd₂(dba)₃), 2,2′-Bis(diphenylphosphino)-1,1′-binaphtyl (BINAP), and sodium-tert-butoxide (NaOtBu) or cesium carbonate (Cs₂CO₃) in an organic solvent, such as toluene, at a suitable temperature. More detailed examples of “A moiety” preparation are described below.

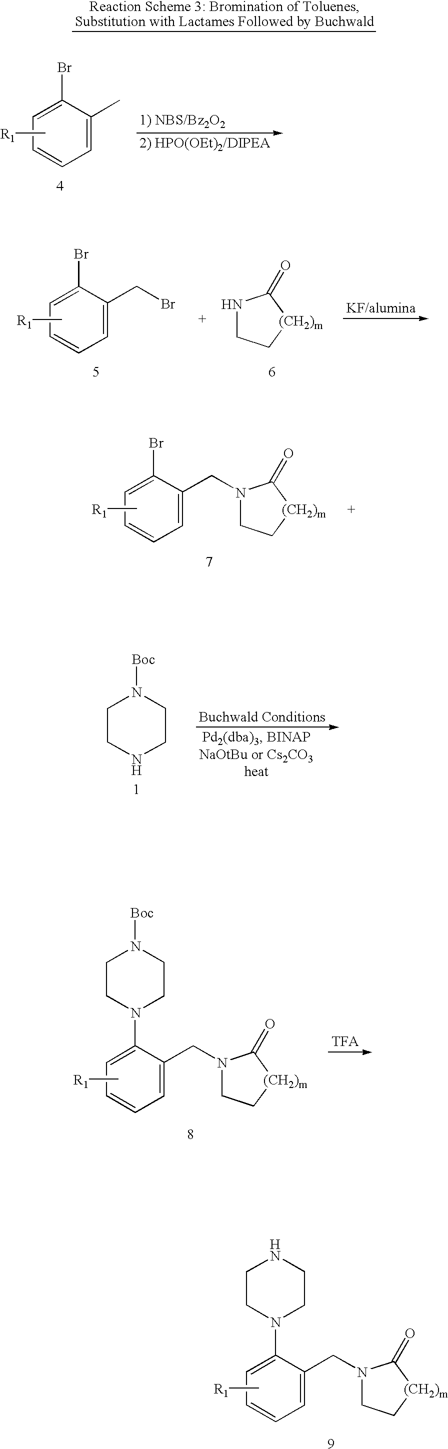

As shown in Reaction Scheme 3, the “A moiety” of the compounds of the present invention can be prepared by reacting various methyl benzenes 4 with NBS in the presence of a radical starter, such as Bz₂O₂, followed by reaction with diethyl phosphite in the presence of a base, such as DIPEA, to give benzylbromides 5, which can then be used to alkylate lactames like 6, in the presence of an appropriate base, such as KF/alumina. The substituted bromobenzenes can then be subjected to Buchwald conditions, followed by deprotection using an appropriate reactant, such as TFA.

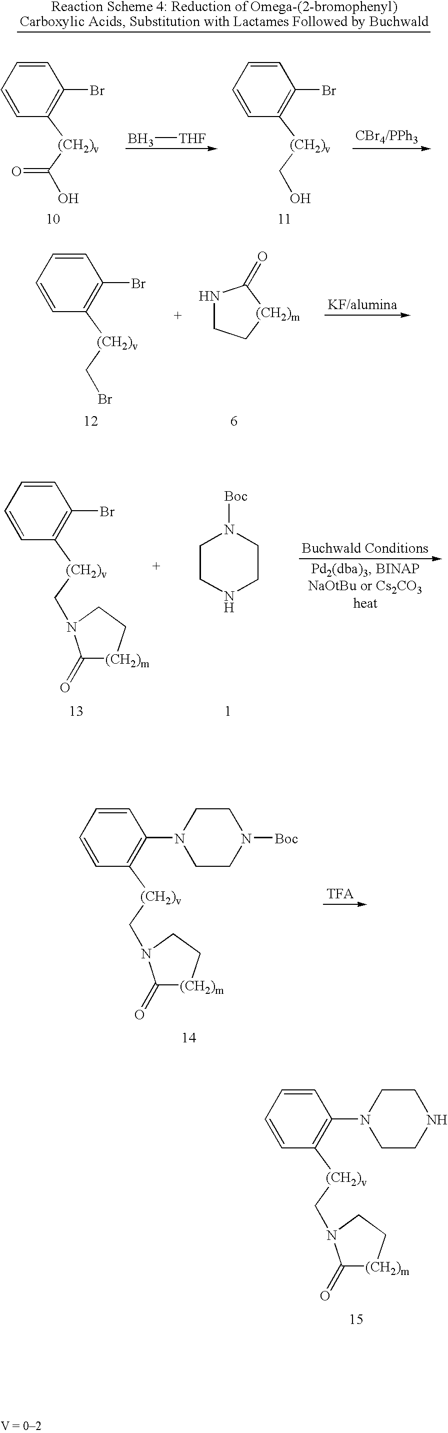

As shown in Reaction Scheme 4, carboxylic acids 10 can be reduced to the corresponding alcohols 11 using an appropriate reagent such as BH₃-THF, which are subsequently transferred to the corresponding alkyl bromides 12 with reagents such as CBr₄ or PPh₃. The alkyl bromides can then be used to alkylate lactames like 6 in the presence of an appropriate base such as KF/alumina. The substituted bromobenzenes can then be subjected to Buchwald conditions followed by deprotection using an appropriate reactant such as TFA.

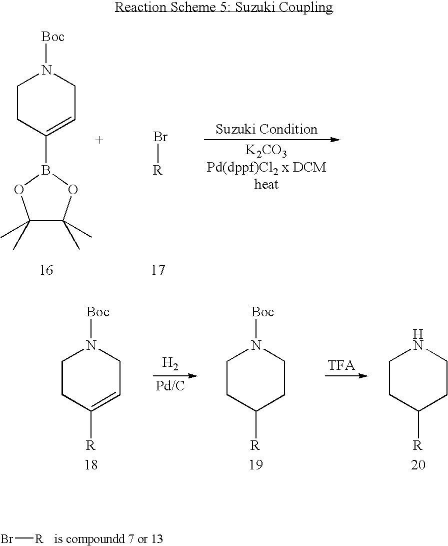

As shown in Reaction Scheme 5,1-(2(H)-pyridine-carboxylic acid-3,6-dihydro-4-(4,4,5,5-tetramethyl-1,3,2-dioxaborolan-2-yl)-1,1-dimethyl ethyl ester 16 (Tetrahedron Lett. 2000, 41, 3705-3708) can be reacted with haloaromates such as 7 or 13 in the presence of a base such as K₂CO₃ and a catalyst such as dichloro(1,1′-bis(diphenylphosphino)-ferrocene)palladium(II) DCM adduct in an organic solvent such as DMF at a suitable temperature. The tetrahydropyridines can be hydrogenated in the presence of a catalyst such as Pd/C to yield the protected piperidines 19 which can subsequently be deprotected with a reagent such as TFA to yield piperidines 20.
Reaction Schemes for Preparation of “C moiety”

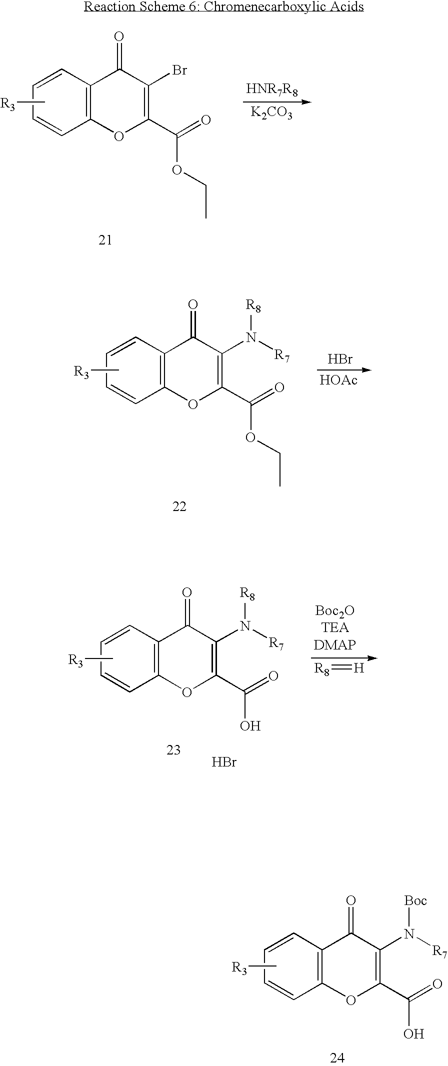

As shown in Reaction Scheme 6, ethyl 3-bromo-4-oxochromene-2-carboxylate 21 (J. Chem. Soc. Perkin Trans. I 1986, 1643-1649) can be reacted with amines with or without a base such as K₂CO₃ in an appropriate solvent such as MeCN to form products 22 which are subsequently treated with a reagent such as HBr/HOAc to form carboxylic acids 23. When R₈ is hydrogen, the free amine can be protected with a reagent such as Boc₂O in the presence of TEA and DMAP in an appropriate solvent.

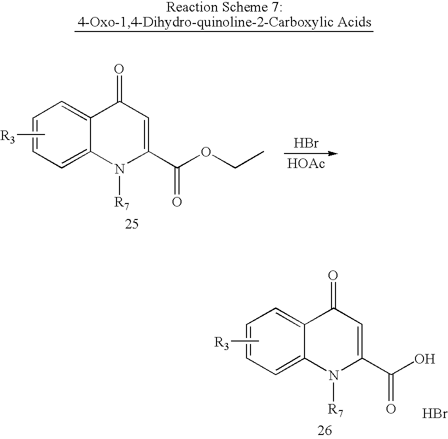

As shown in Reaction Scheme 7, ethyl 4-oxo-1,4-dihydro-quinoline-2-carboxylates 25 (Bioorg. Med. Chem. Lett. 2000, 10, 1487-1490) can be converted into the corresponding acids 26 by an appropriate reactant such as HBr/HOAc.

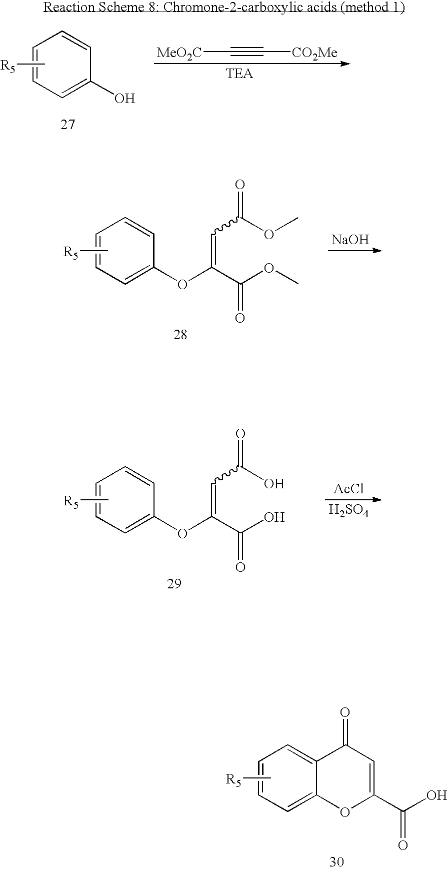

As shown in Reaction Scheme 8, substituted phenols 27 can be reacted with triethylamine followed by dimethyl acetylendicarboxylate in diethyl ether to yield compounds 28 (Aust J. Chem. 1995, 48, 677-686). Saponification of the latter with aqueous sodium hydroxide leads to acids 29 which are subsequently cyclized to the chromone-2-carboxylic acids 30 using concentrated sulfuric acid in acetyl chloride.

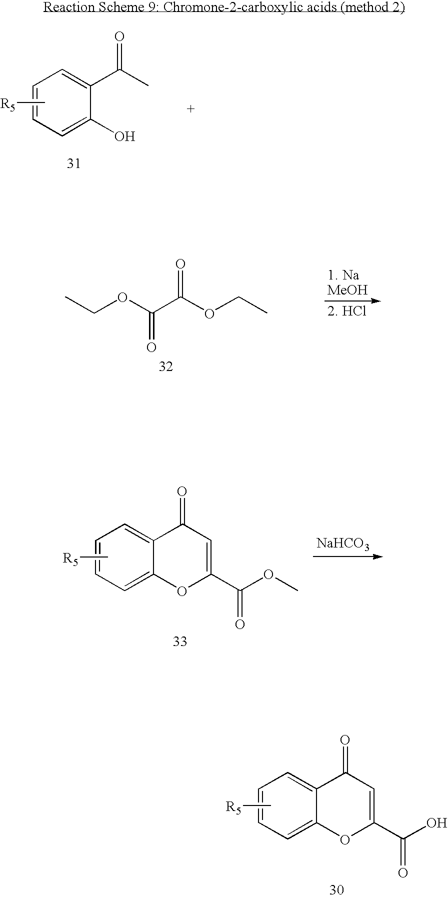

As shown in Reaction Scheme 9, 2′-hydroxyacetophenones 31 can be reacted with diethyl oxalate 32 in the presence of a base such as sodium methoxide in an appropriate solvent such as methanol or benzene followed by treatment with an acid such as hydrochloric acid to yield chromone-2-carboxylic acid esters 33 (J. Indian Chem. Soc. 1986, 63, 600-602). The esters can be cleaved using basic conditions such as sodium bicarbonate in water or acidic conditions such as polyphosphoric acid at an appropriate temperature to the corresponding acids 30.

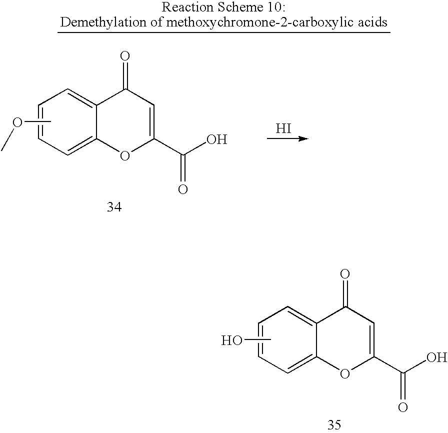

As shown in Reaction Scheme 10, methoxy-substituted chromone-2-carboxylic acids can be demethylated with reagents such as hydroiodic acid in an appropriate solvent such as glacial acetic acid to yield the corresponding hydroxy-substituted chromone-2-carboxylic acids.

5,7-Dihydroxychromone-2-carboxylic acid was prepared as described in the literature (OPPI Briefs 1991, 23, 390-392).

The following describes the detailed examples of the invention.

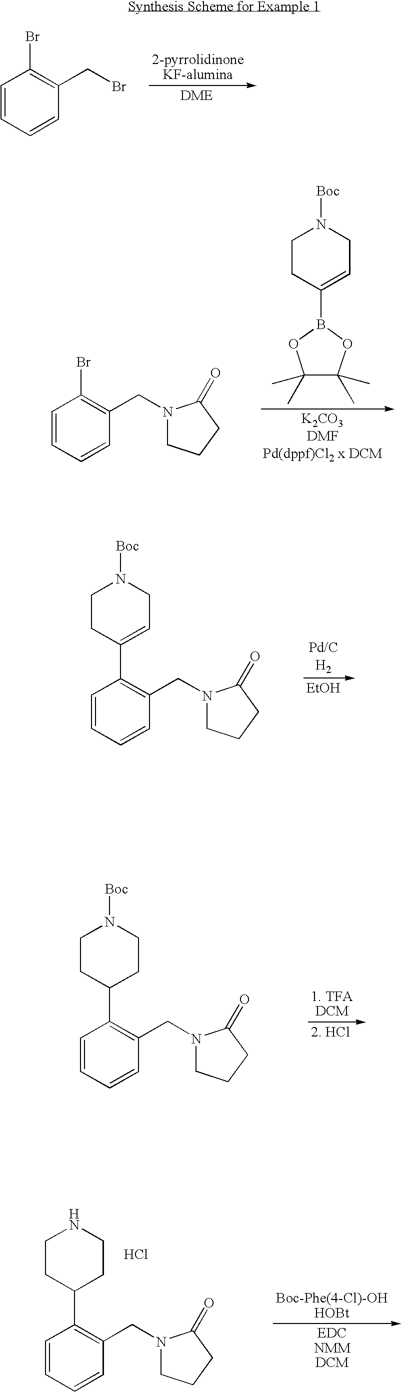

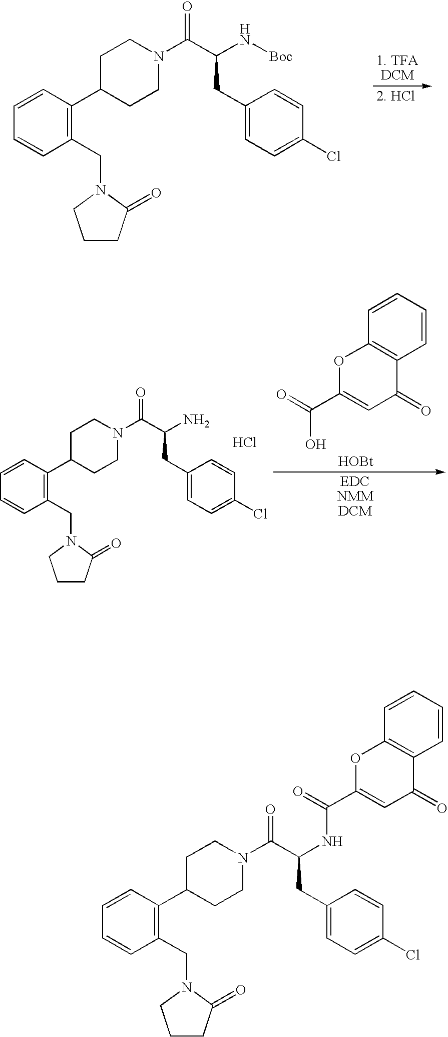

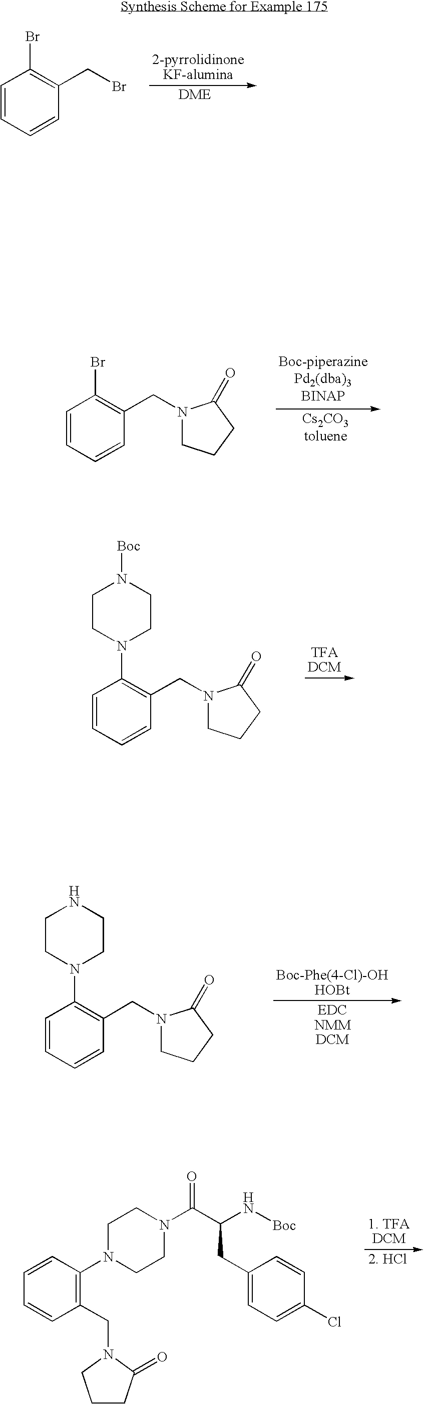

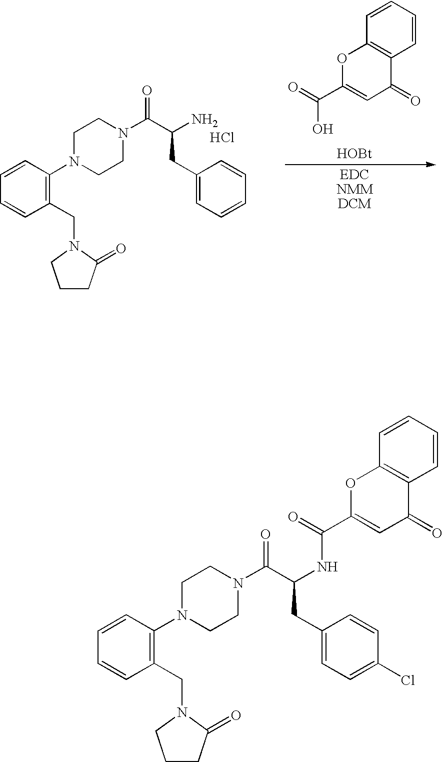

The following examples are provided to illustrate the invention and are not limiting the scope of the invention in any manner.

### EXAMPLE 1

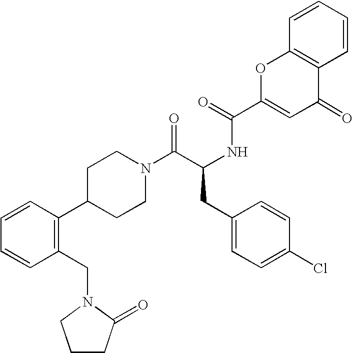

To chromone-2-carboxylic acid (16 mg) in DCM (2 ml) was added intermediate 1f) (36 mg), N-methylmorpholine (14 μl), HOBt (14 mg) and stirred for 20 min. EDC (23 mg) was added and stirring was continued for 1 h. An additional amount of N-methylmorpholine (8 μl) was added and stirred overnight. The reaction mixture was poured into water (5 ml) and the organic phase was separated. The aqueous phase was extracted two times with ethyl acetate. The combined organic phases were washed three times with 0.5 N HCl and three times with saturated sodium bicarbonate solution, dried over Na₂SO₄ and concentrated to yield the product which was purified by column chromatography.

white solid

R_{f}=0.19 (ethyl acetate); Mp. 133-139° C.

The required intermediates can be synthesized in the following way:

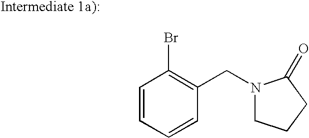

To a solution of 2-bromobenzyl bromide (3.05 g) and 2-pyrrolidinone (0.85 g) in DME (20 ml) was added KF-alumina (0.45 g) and the mixture was stirred for 48 h at room temperature. The inorganics were filtered off and the solvent was removed to afford the desired compound.

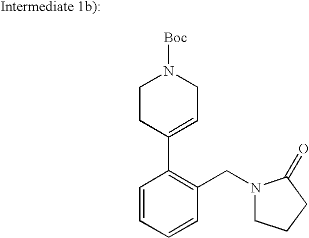

To intermediate 1a) (623 mg) in DMF (20 ml) was added 1-(2(H)-pyridine-carboxylic acid-3,6-dihydro-4-(4,4,5,5-tetramethyl-1,3,2-dioxaborolan-2-yl)-1,1-dimethyl ethyl ester (909 mg), dichloro(1,1′-bis(diphenylphosphino)-ferrocene)palladium(II) DCM adduct (108 mg) and K₂CO₃ (1002 mg). The reaction was heated to about 90° C. overnight. The mixture was cooled, diluted with DCM and filtered through Celite. The filtrate was concentrated to dryness and the resulting residue was taken up in EtOAc (50 ml). The organics were washed with water, brine and concentrated to dryness. The crude product was purified by flash chromatography.

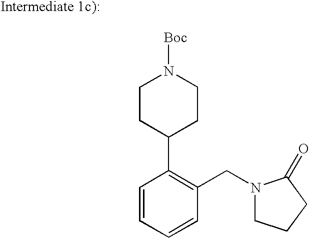

To intermediate 1b) (422 mg) in EtOH (20 ml) was added a slurry of 10% Pd/C in EtOH (20 ml). The mixture was stirred rapidly under H₂ (1 atm) for about 2 h. The reaction mixture was filtered over a pad of Celite and washed with EtOAc (100 ml). The filtrate was concentrated to dryness to yield the final compound.

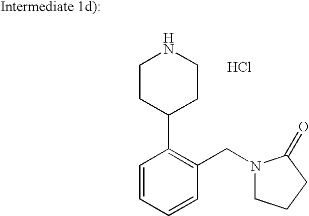

To the Boc-protected amine from 1c) (190 mg) in DCM (5 ml) was added TFA (1 ml) and stirred at room temperature for 90 min. Additional TFA (1 ml) was added and stirred for 10 min. The reaction mixture was diluted with DCM (10 ml) and carefully basified by pouring into 10% aqueous sodium carbonate solution (20 ml). The organic layer was separated and the aqueous layer was further extracted three times with DCM. The combined organics were washed with water and brine, dried over Na₂SO₄, concentrated to give a white solid.

For prolonged storage, the free base was converted into the corresponding hydrochloride. The free base was dissolved in DCM (5 ml) and app. 1 M HCl in ether (10 ml) was added. The precipitate was filtered and the residue was washed three times with ether and dried under reduced pressure to yield the desired compound.

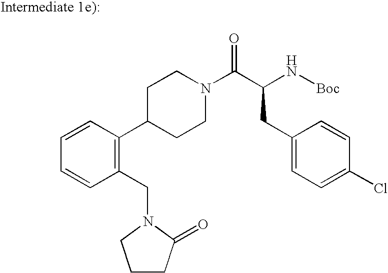

To Boc-L-4-chlorophenylalanine (82 mg) in DCM (5 ml) was added the amine hydrochloride from 1d) (42 mg), N-methylmorpholine (42 μl), HOBt (48 mg) and stirred for 20 min. EDC (72 mg) was added and stirring was continued for 1 h. An additional amount of N-methylmorpholine (20 μl) was added and stirred overnight. The reaction mixture was poured into water (5 ml) and the organic phase was separated. The aqueous phase was extracted two times with DCM. The combined organic phases were washed with 0.5 N HCl and saturated sodium bicarbonate solution, dried over Na₂SO₄ and concentrated in vacuo. Purification by column chromatography yielded the title compound.

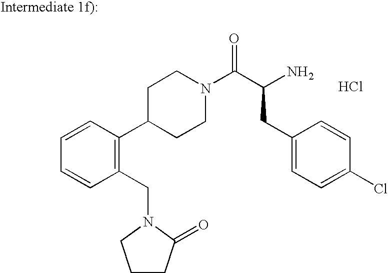

To the Boc-protected amine from 1e) (154 mg) in DCM (5 ml) was added TFA (1 ml) and stirred at room temperature for 90 min. Additional TFA (1 ml) was added and stirred for 10 min. The reaction mixture was diluted with DCM (10 ml) and carefully basified by pouring into 10% aqueous sodium carbonate solution (20 ml). The organic layer was separated and the aqueous layer was further extracted three times with DCM. The combined organics were washed with water and brine, dried over Na₂SO₄, concentrated to give a white solid.

For prolonged storage, the free base was converted into the corresponding hydrochloride. The free base was dissolved in DCM (5 ml) and app. 1 M HCl in ether (10 ml) was added. The precipitate was filtered and the residue was washed three times with ether and dried under reduced pressure to yield the desired compound.

The following examples can be prepared in a similar way:

### EXAMPLE 2

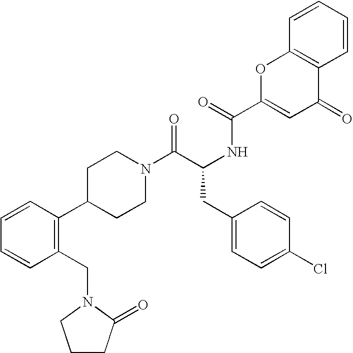

white solid

R_{f}=0.19 (ethyl acetate); Mp. 133-139° C.

### EXAMPLE 3

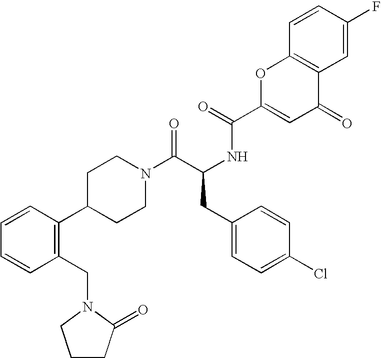

white solid

R_{f} 0.24 (ethyl acetate); Mp. 135-140° C.

### EXAMPLE 4

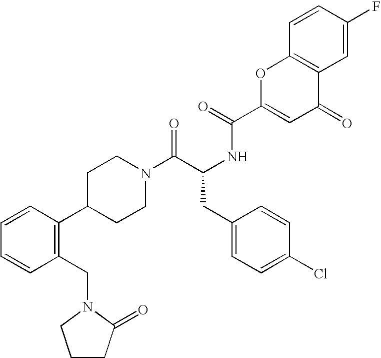

white solid

R_{f} 0.24 (ethyl acetate); Mp. 132-138° C.

### EXAMPLE 5

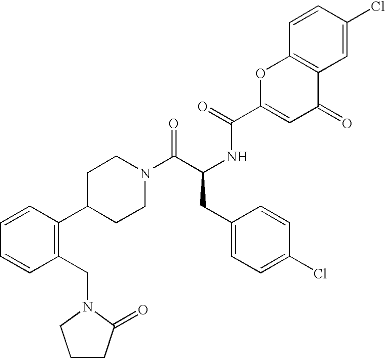

white solid

R_{f} 0.26 (ethyl acetate); Mp. 145-150° C.

### EXAMPLE 6

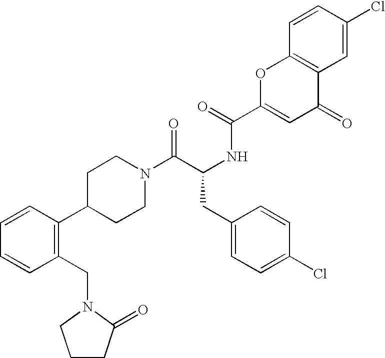

white solid

R_{f}=0.26 (ethyl acetate); Mp. 150-155° C.

### EXAMPLE 7

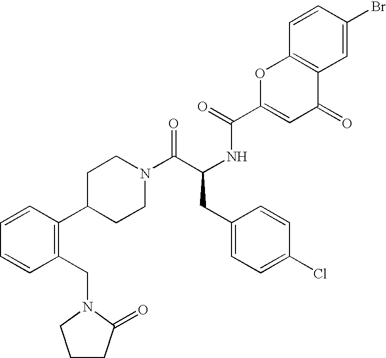

white solid

R_{f}=0.27 (ethyl acetate); Mp. 140-145° C.

### EXAMPLE 8

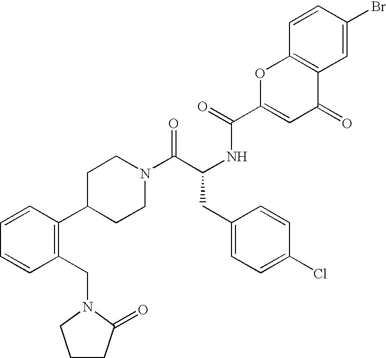

white solid

R_{f}=0.27 (ethyl acetate); Mp. 135-141° C.

### EXAMPLE 9

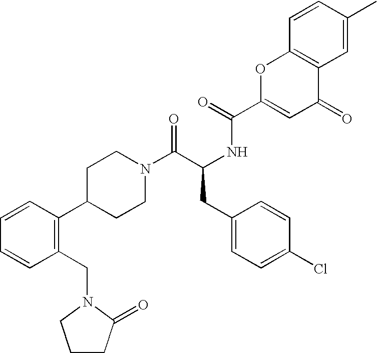

white solid

R_{f}=0.21 (ethyl acetate); Mp. 130-135° C.

### EXAMPLE 10

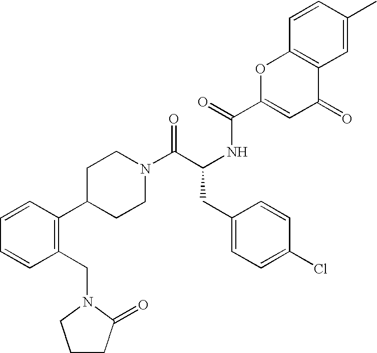

white solid

R_{f}=0.21 (ethyl acetate); Mp. 121-127° C.

### EXAMPLE 11

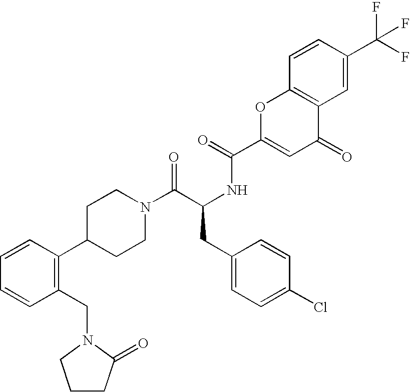

white solid

R_{f}=0.30 (ethyl acetate); Mp. 135-145° C.

### EXAMPLE 12

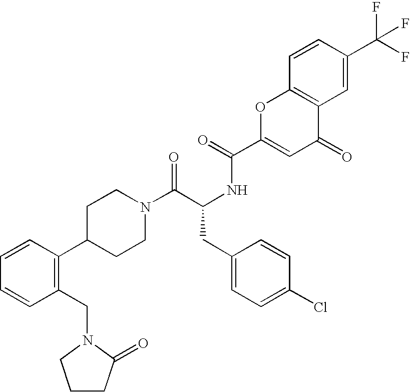

white solid

R_{f} 0.27 (ethyl acetate); Mp. 145-155° C.

### EXAMPLE 13

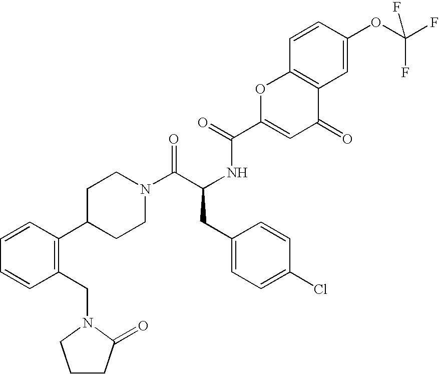

white solid

R_{f} 0.25 (ethyl acetate); Mp. 115-130° C.

### EXAMPLE 14

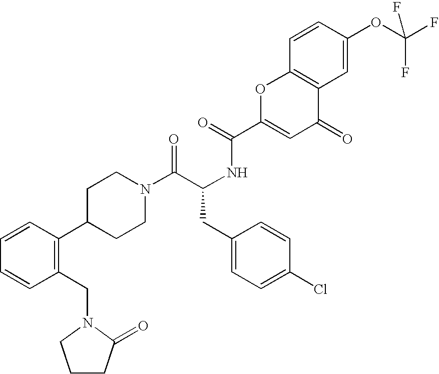

white solid

R_{f}=0.25 (ethyl acetate); Mp. 115-130.

### EXAMPLE 15

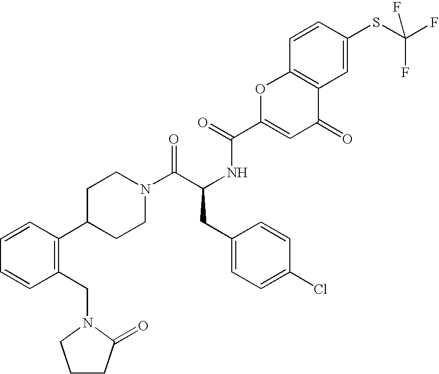

white solid

R_{f}=0.23 (ethyl acetate).

### EXAMPLE 16

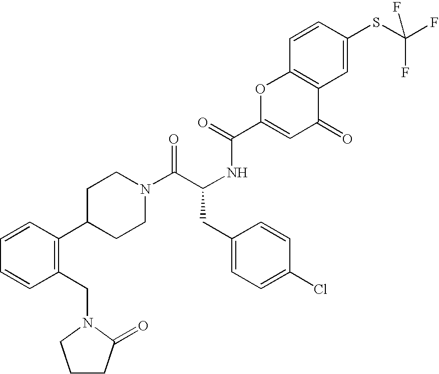

white solid

R_{f}=0.23 (ethyl acetate).

### EXAMPLE 17

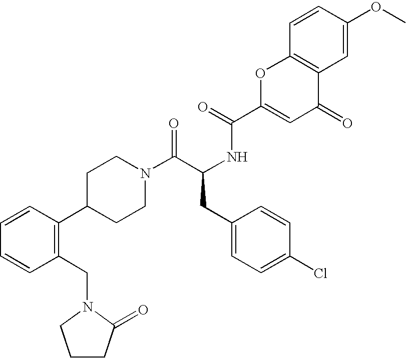

white solid

R_{f}=0.19 (ethyl acetate); Mp. 135-145° C.

### EXAMPLE 18

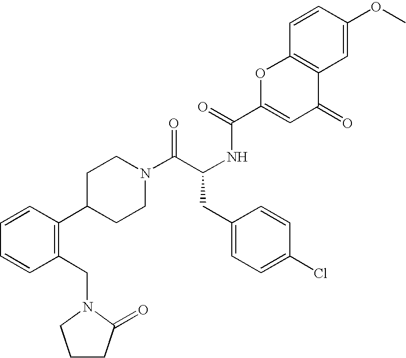

white solid

R_{f}=0.17 (ethyl acetate); Mp. 140-150° C.

### EXAMPLE 19

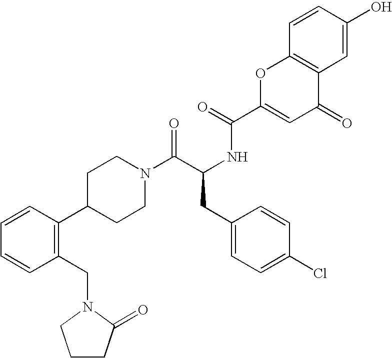

white solid

R_{f}=0.14 (DCM/methanol 95:5).

### EXAMPLE 20

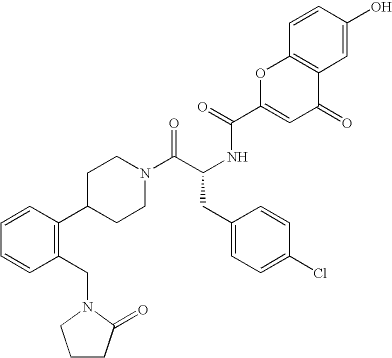

white solid

R_{f} =0.14 (DCM/methanol 95:5).

### EXAMPLE 21

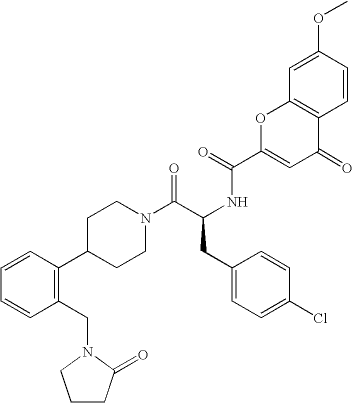

white solid

R_{f} =0.10 (ethyl acetate); Mp. 125-140° C.

### EXAMPLE 22

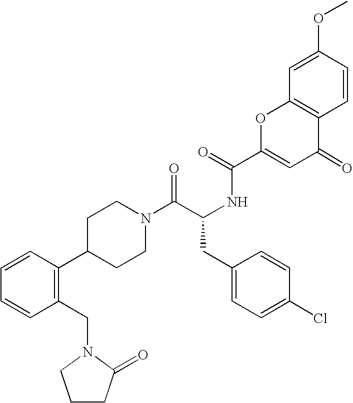

white solid

R_{f}=0.10 (ethyl acetate); Mp. 125-140° C.

### EXAMPLE 23

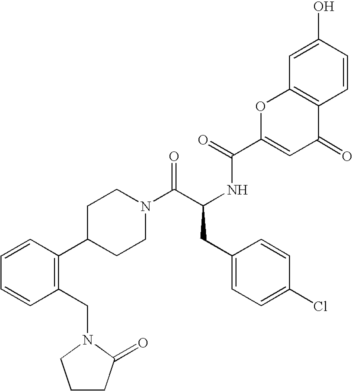

white solid

R_{f}=0.14 (DCM/methanol 95:5).

### EXAMPLE 24

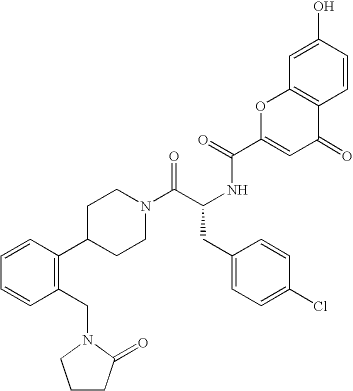

white solid

R_{f} =0.14 (DCM/methanol 95:5).

### EXAMPLE 25

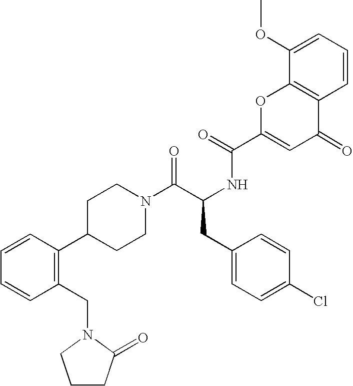

white solid

R_{f} =0.09 (ethyl acetate); Mp. 120-125.

### EXAMPLE 26

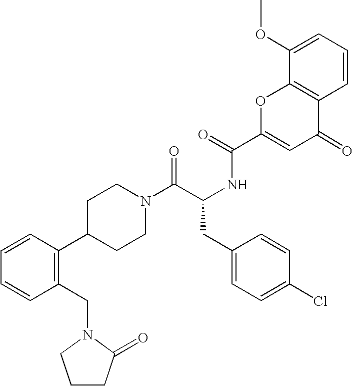

white solid

R_{f} =0.09 (ethyl acetate); Mp. 120-125.

### EXAMPLE 27

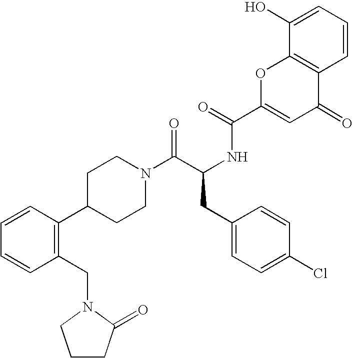

white solid

R_{f} =0.05 (ethyl acetate); Mp. 165-170° C.

### EXAMPLE 28

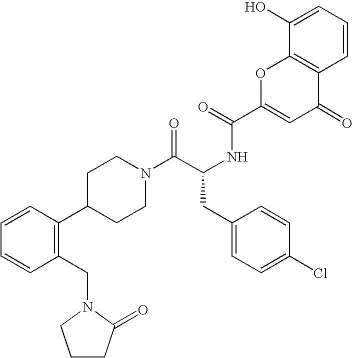

white solid

R_{f}=0.05 (ethyl acetate); Mp. 165-170° C.

### EXAMPLE 29

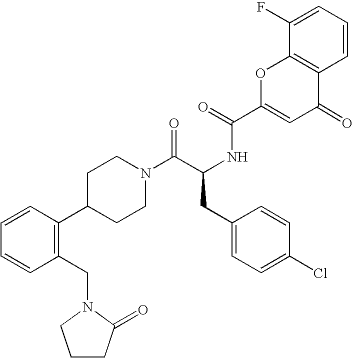

white solid

R_{f}=0.14 (ethyl acetate).

### EXAMPLE 30

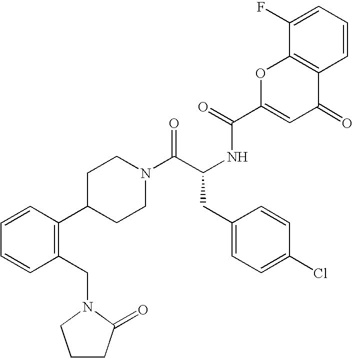

white solid

R_{f}=0.14 (ethyl acetate).

### EXAMPLE 31

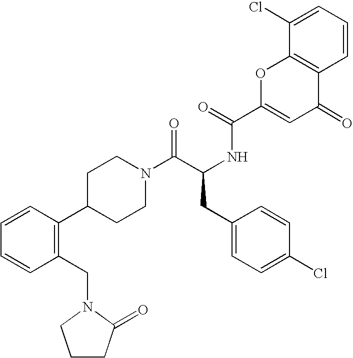

white solid

R_{f}=0.16 (ethyl acetate).

### EXAMPLE 32

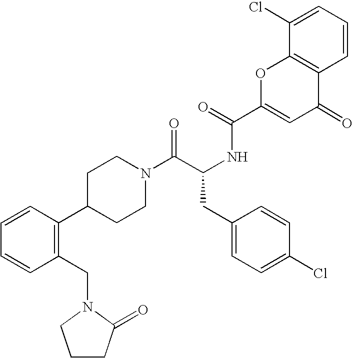

white solid

R_{f}=0.16 (ethyl acetate).

### EXAMPLE 33

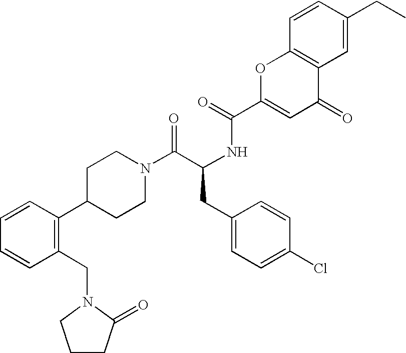

white solid

R_{f}=0.10 (ethyl acetate).

### EXAMPLE 34

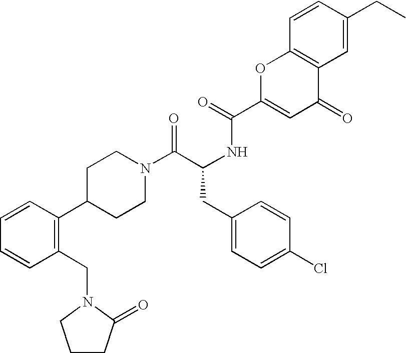

white solid

R_{f}=0.10 (ethyl acetate).

### EXAMPLE 35

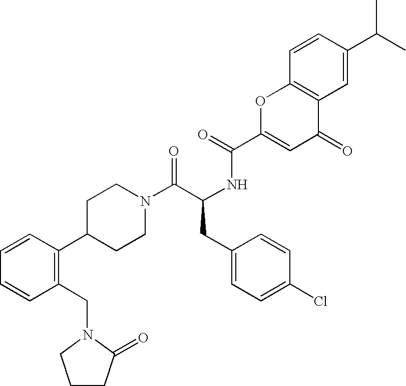

white solid

R_{f}=0.12 (ethyl acetate).

### EXAMPLE 36

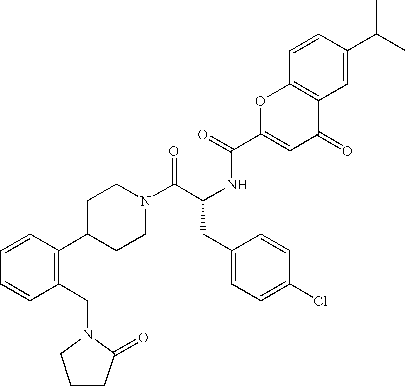

white solid

R_{f}=0.12 (ethyl acetate).

### EXAMPLE 37

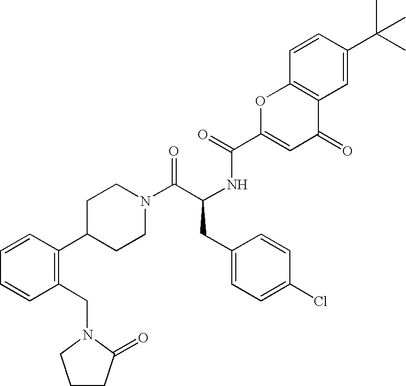

white solid

R_{f}=0.14 (ethyl acetate); Mp. 140-145° C.

### EXAMPLE 38

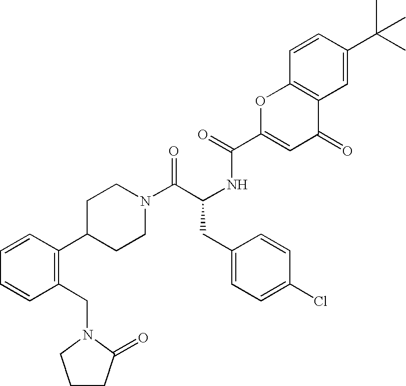

white solid

R_{f}=0.14 (ethyl acetate); Mp. 140-145° C.

### EXAMPLE 39

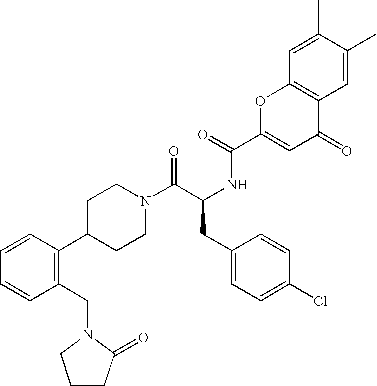

white solid

R_{f}=0.12 (ethyl acetate); Mp. 135-140° C.

### EXAMPLE 40

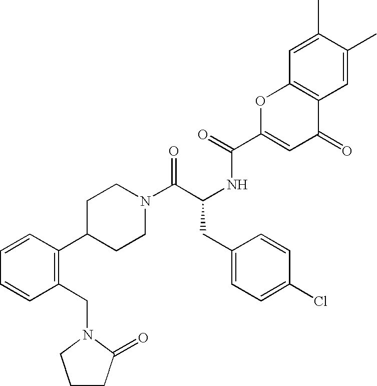

white solid

R_{f}=0.12 (ethyl acetate); Mp. 135-140° C.

### EXAMPLE 41

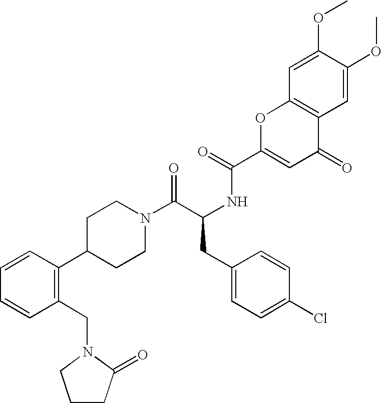

white solid

R_{f}=0.03 (ethyl acetate); Mp. 135-150° C.

### EXAMPLE 42

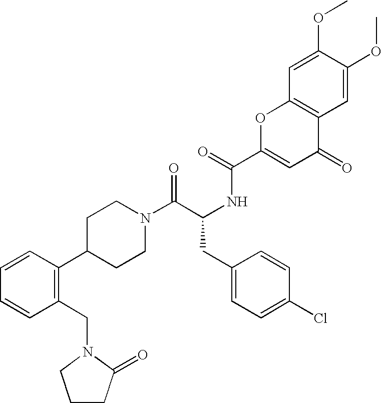

white solid

R_{f}=0.03 (ethyl acetate); Mp. 135-150° C.

### EXAMPLE 43

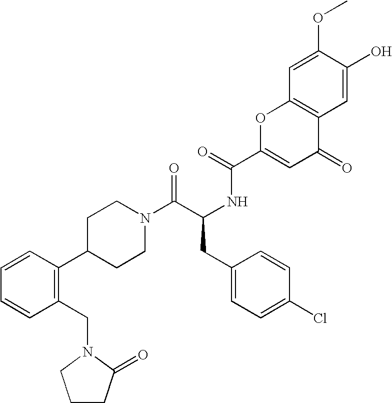

white solid

R_{f}=0.19 (DCM/methanol 95:5).

### EXAMPLE 44

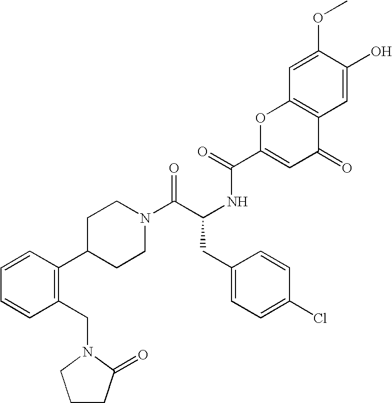

white solid

R_{f}=0.19 (DCM/methanol 95:5).

### EXAMPLE 45

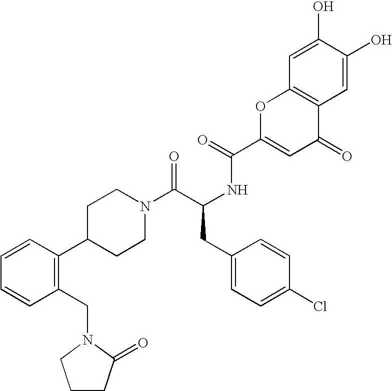

yellow solid

R_{f}=0.01 (DCM/methanol 95:5).

### EXAMPLE 46

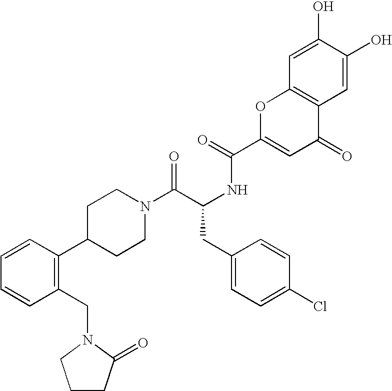

yellow solid

R_{f}=0.01 (DCM/methanol 95:5).

### EXAMPLE 47

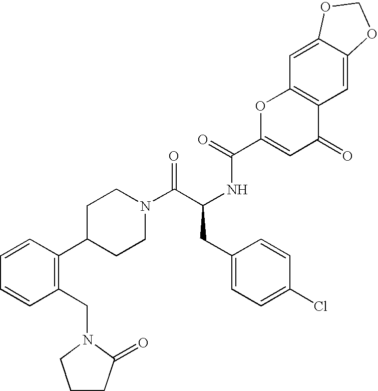

white solid

R_{f}=0.07 (ethyl acetate); Mp. 140-155° C.

### EXAMPLE 48

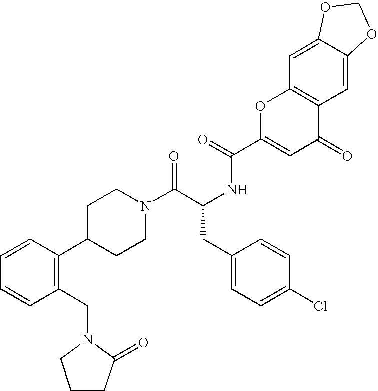

white solid

R_{f}=0.07 (ethyl acetate); Mp. 140-155° C.

### EXAMPLE 49

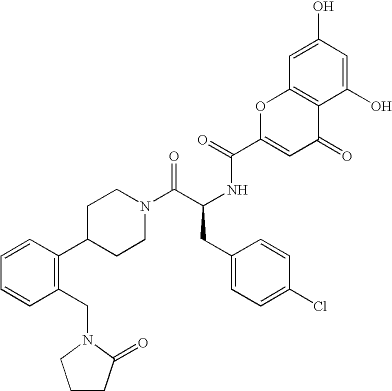

### EXAMPLE 50

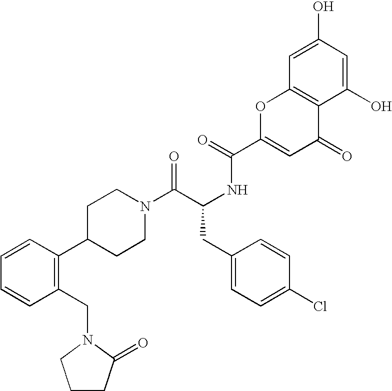

### EXAMPLE 51

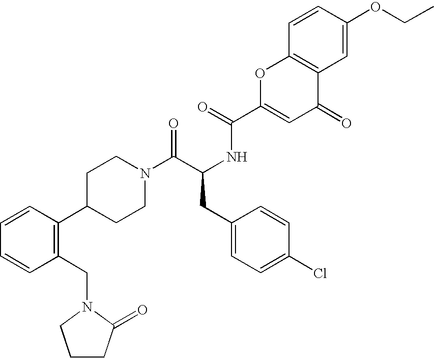

white solid

R_{f}=0.10 (ethyl acetate). Mp. 110-125° C.

### EXAMPLE 52

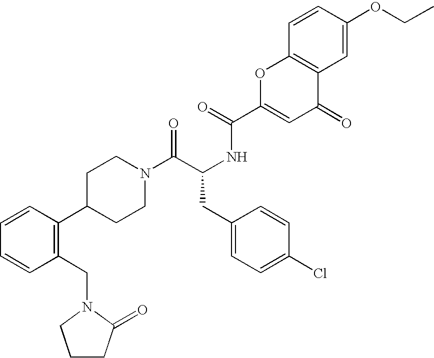

white solid

R_{f}=0.10 (ethyl acetate). Mp. 110-125° C.

### EXAMPLE 53

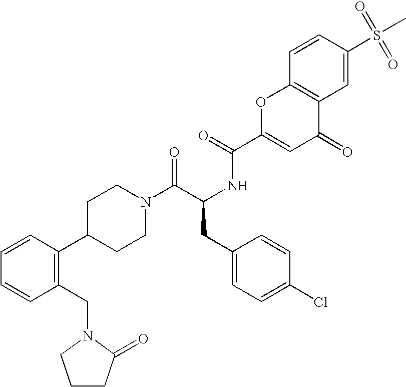

white solid

R_{f}=0.03 (ethyl acetate). Mp. 150-160° C.

### EXAMPLE 54

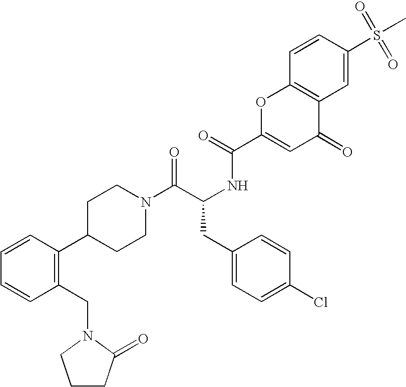

white solid

R_{f}=0.03 (ethyl acetate). Mp. 150-160° C.

### EXAMPLE 55

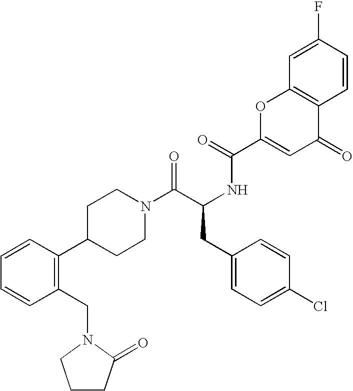

white solid

R_{f}=0.12 (ethyl acetate). Mp. 120-130° C.

### EXAMPLE 56

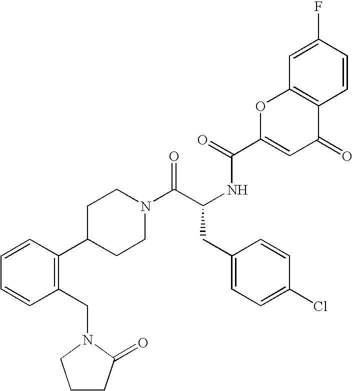

white solid

R_{f}=0.12 (ethyl acetate). Mp. 120-130° C.

### EXAMPLE 57

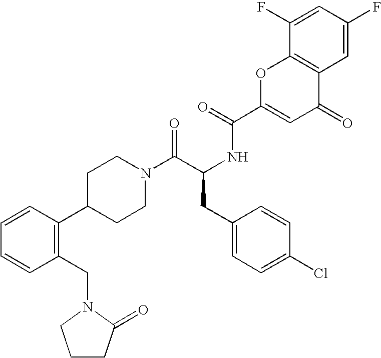

### EXAMPLE 58

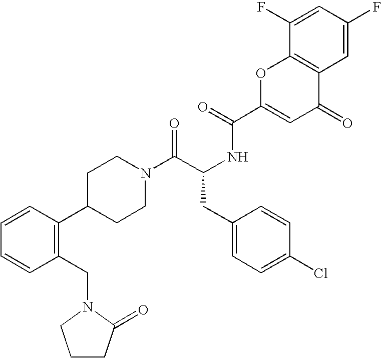

### EXAMPLE 59

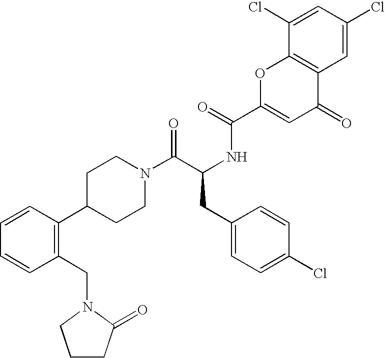

### EXAMPLE 60

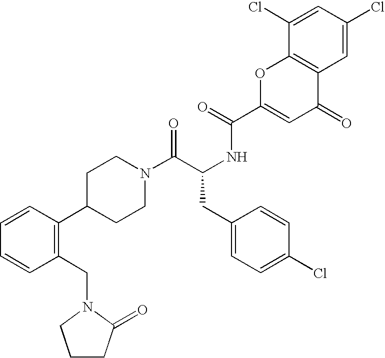

### EXAMPLE 61

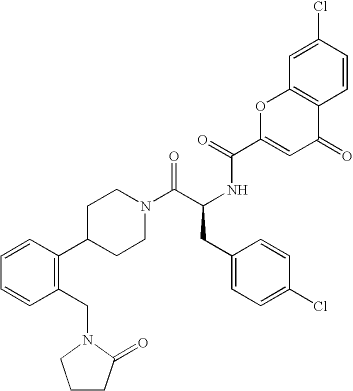

### EXAMPLE 62

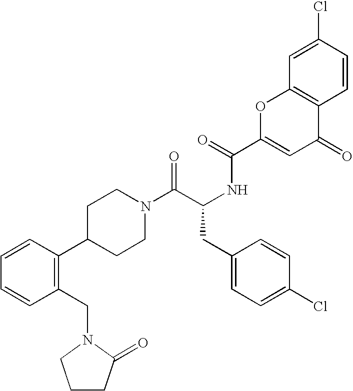

### EXAMPLE 63

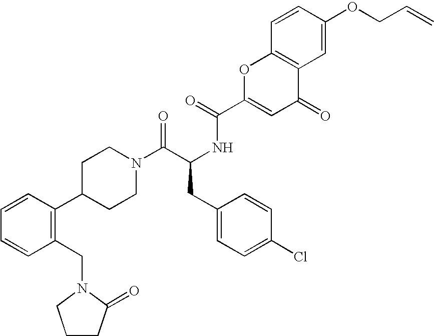

### EXAMPLE 64

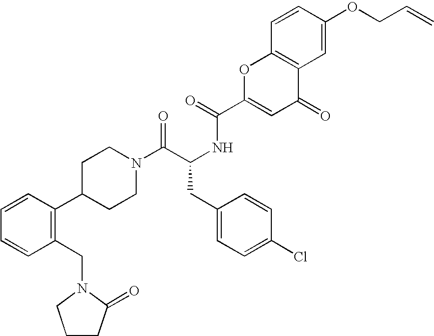

### EXAMPLE 65

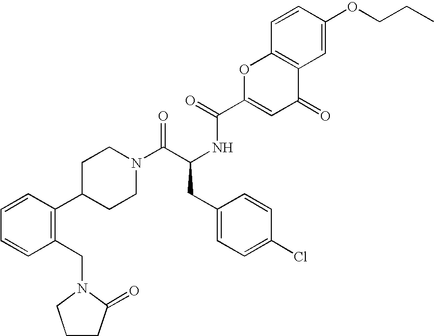

### EXAMPLE 66

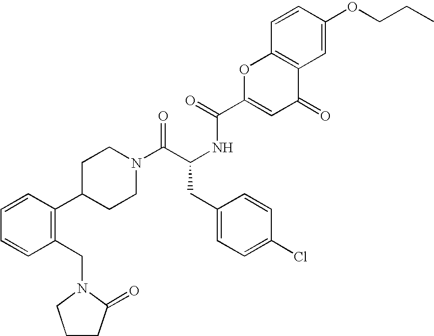

### EXAMPLE 67

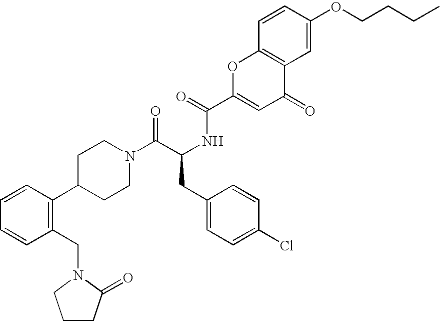

### EXAMPLE 68

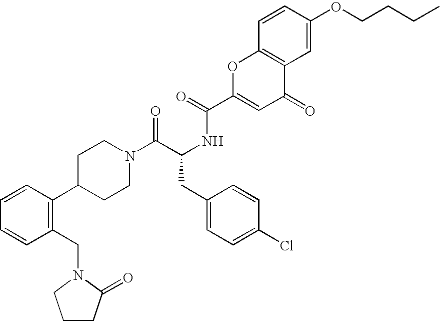

### EXAMPLE 69

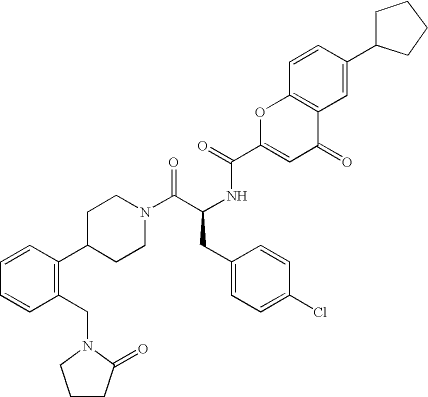

### EXAMPLE 70

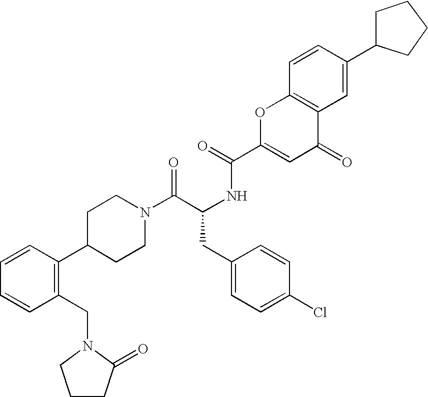

### EXAMPLE 71

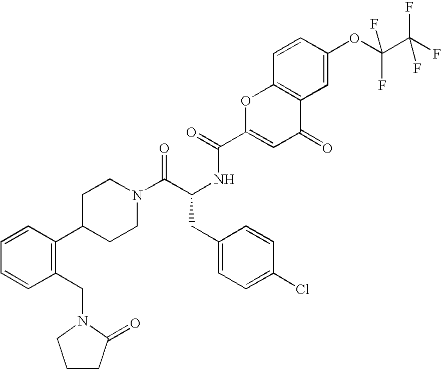

### EXAMPLE 72

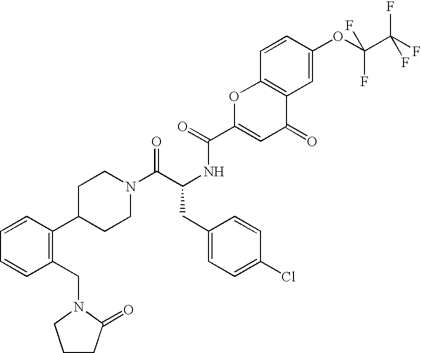

### EXAMPLE 73

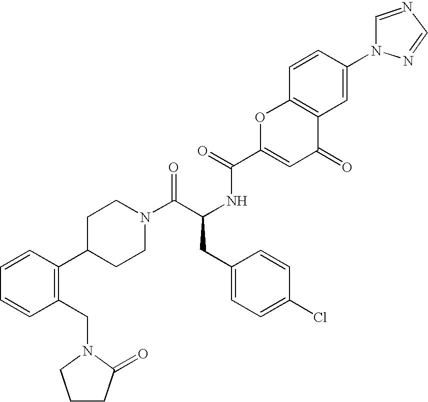

### EXAMPLE 74

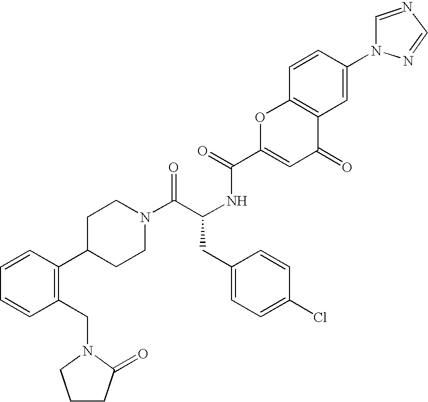

### EXAMPLE 75

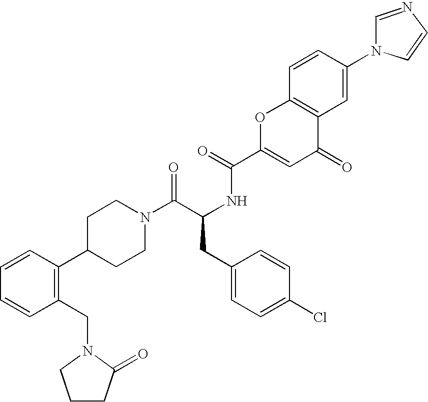

### EXAMPLE 76

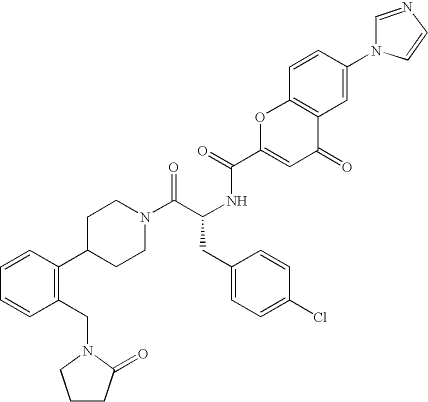

### EXAMPLE 77

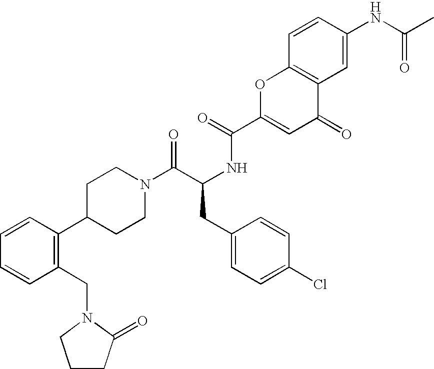

### EXAMPLE 78

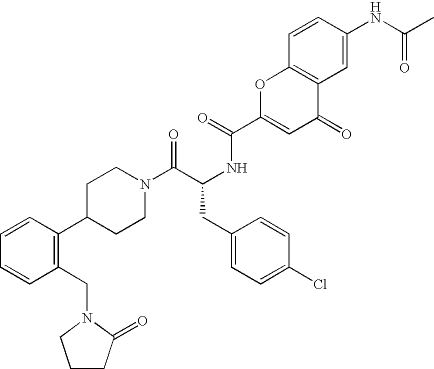

### EXAMPLE 79

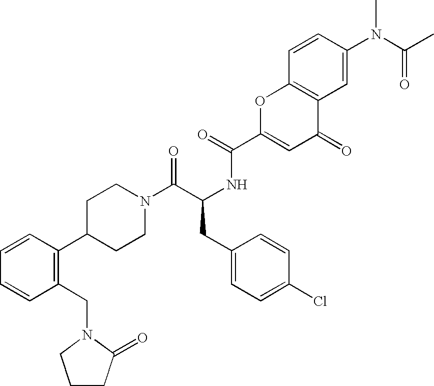

### EXAMPLE 80

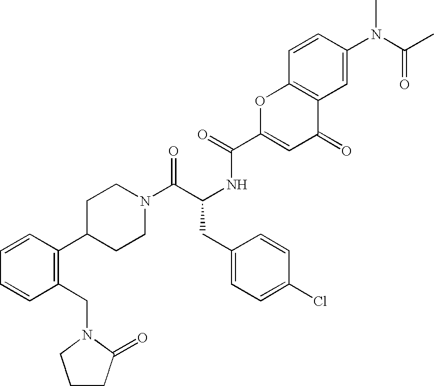

### EXAMPLE 81

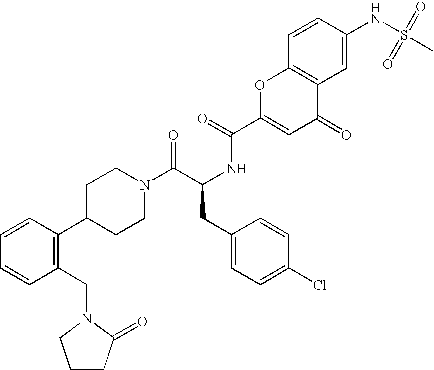

### EXAMPLE 82

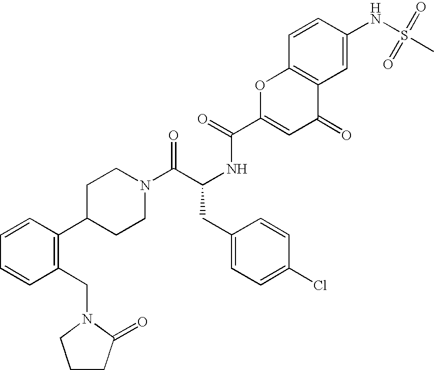

### EXAMPLE 83

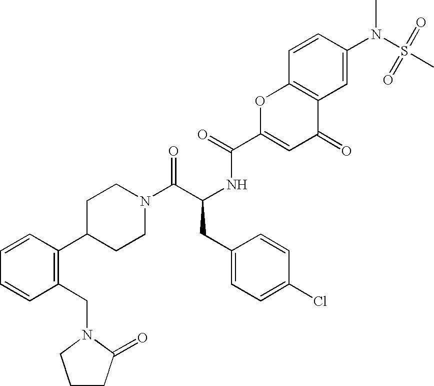

### EXAMPLE 84

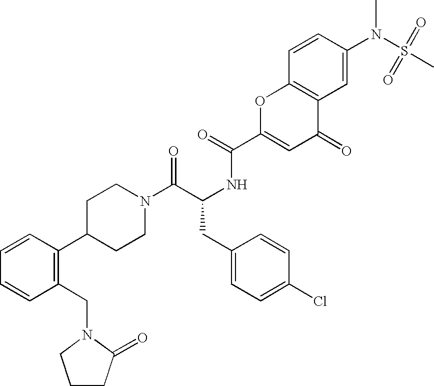

### EXAMPLE 85

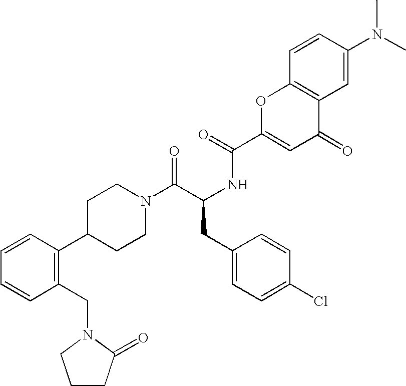

### EXAMPLE 86

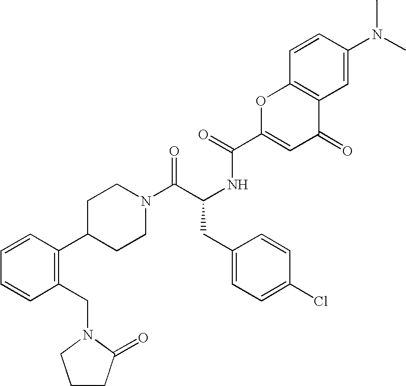

### EXAMPLE 87

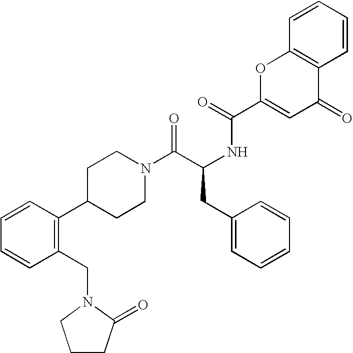

white solid

R_{f}=0.22 (DCM/methanol 95:5).

### EXAMPLE 88

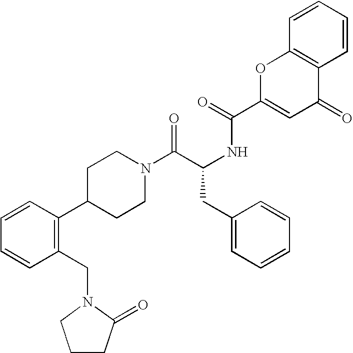

white solid

R_{f}=0.22 (DCM/methanol 95:5).

### EXAMPLE 89

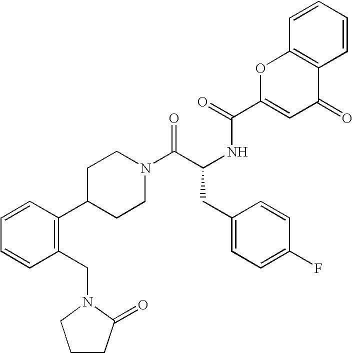

white solid

R_{f}=0.20 (ethyl acetate); Mp. 112-118° C.

### EXAMPLE 90

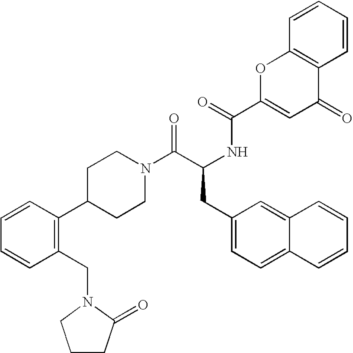

white solid

R_{f}=0.19 (ethyl acetate); Mp. 137-139° C.

### EXAMPLE 91

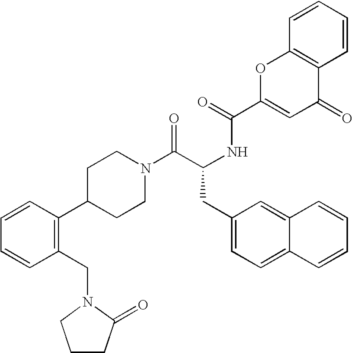

white solid

R_{f}=0.19 (ethyl acetate); Mp. 137-139° C.

### EXAMPLE 92

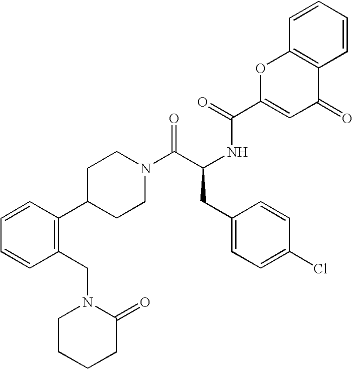

white solid

R_{f}=0.36 (DCM/methanol 95:5).

### EXAMPLE 93

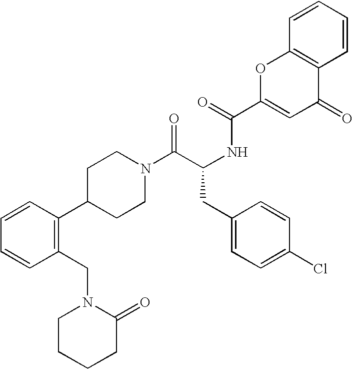

white solid

R_{f}=0.36 (DCM/methanol 95:5).

### EXAMPLE 94

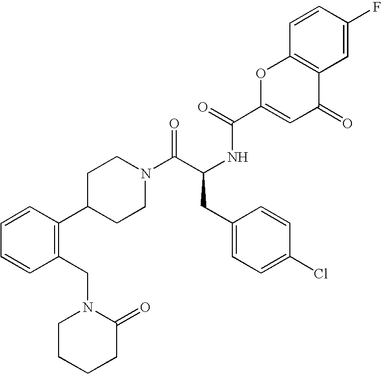

### EXAMPLE 95

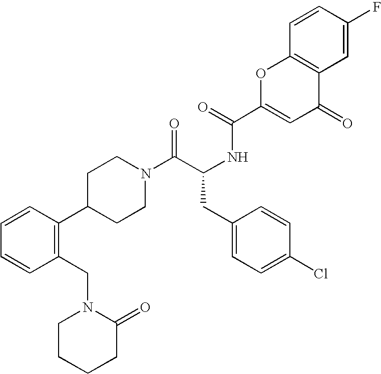

white solid

R_{f}=0.24 (DCM/methanol 95:5).

### EXAMPLE 96

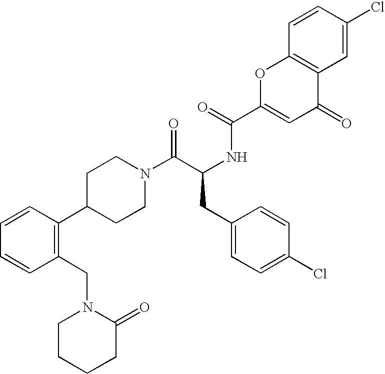

### EXAMPLE 97

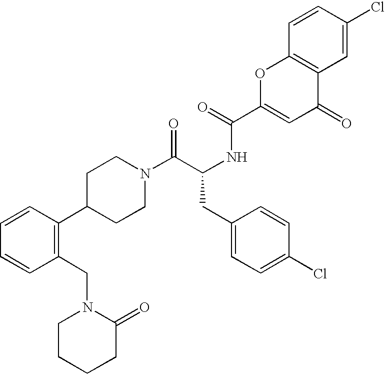

white solid

R_{f}=0.26 (DCM/methanol 95:5).

### EXAMPLE 98

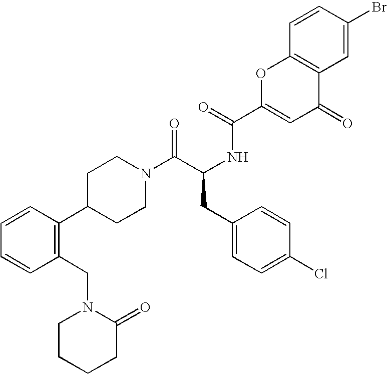

### EXAMPLE 99

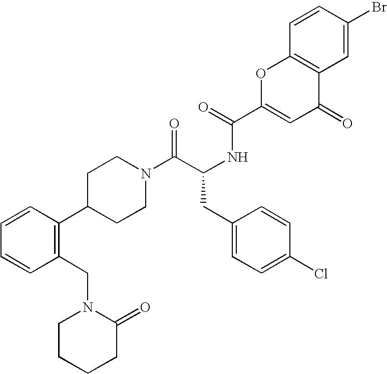

white solid

R_{f}=0.28 (DCM/methanol 95:5).

### EXAMPLE 100

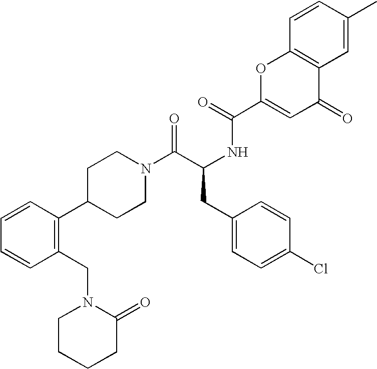

### EXAMPLE 101

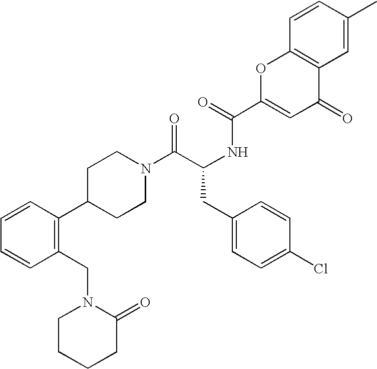

white solid

R_{f}=0.25 (DCM/methanol 95:5).

### EXAMPLE 102

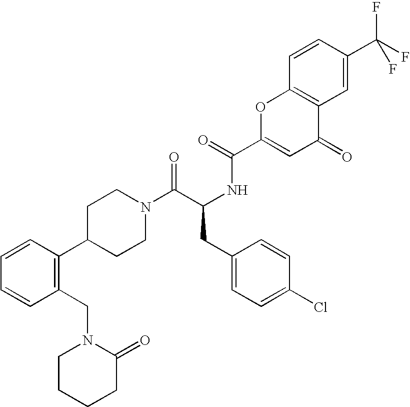

### EXAMPLE 103

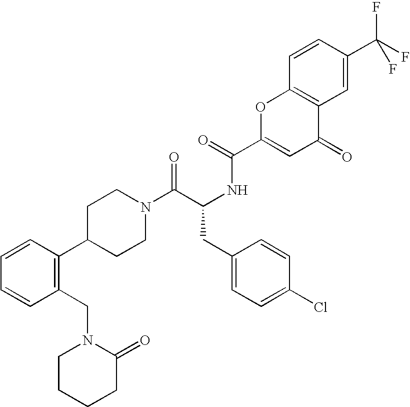

### EXAMPLE 104

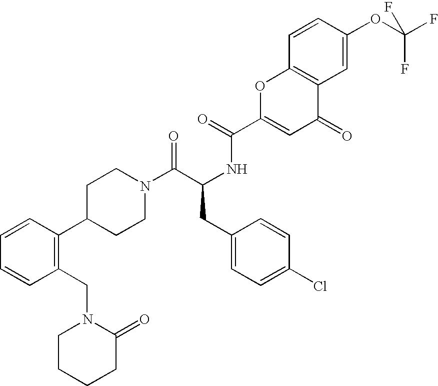

### EXAMPLE 105

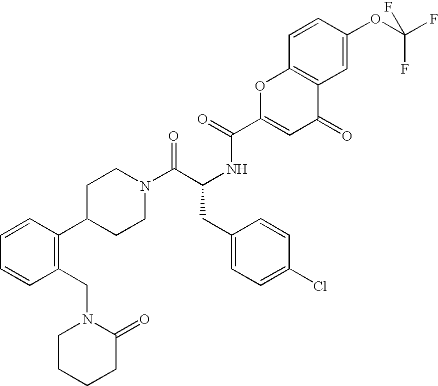

white solid

R_{f}=0.23 (DCM/methanol 95:5).

### EXAMPLE 106

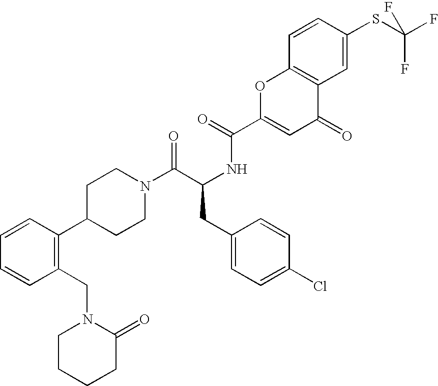

### EXAMPLE 107

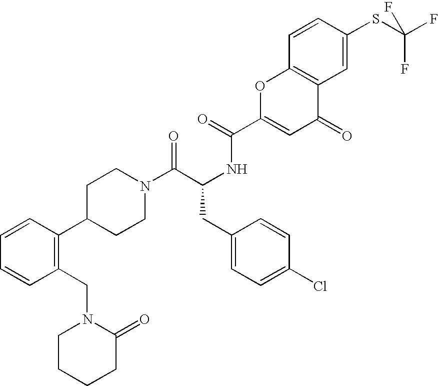

white solid

R_{f}=0.20 (DCM/methanol 95:5).

### EXAMPLE 108

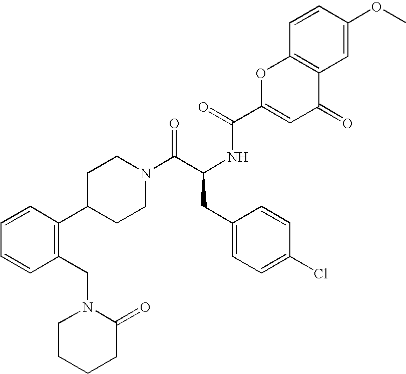

### EXAMPLE 109

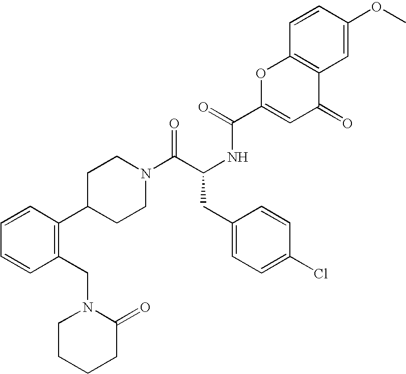

white solid

R_{f}=0.23 (DCM/methanol 95:5).

### EXAMPLE 110

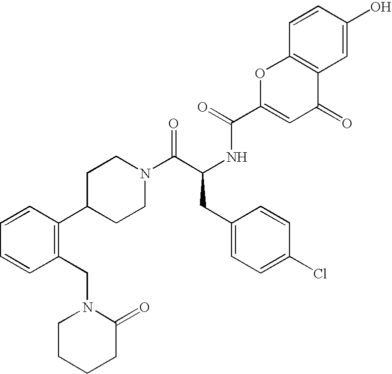

### EXAMPLE 111

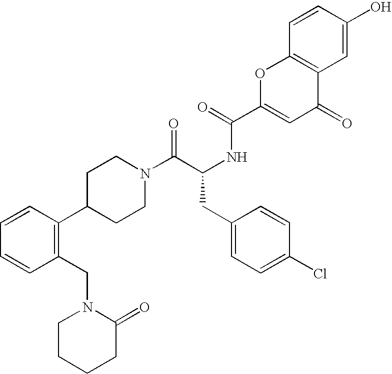

white solid

R_{f}=0.18 (DCM/methanol 95:5).

### EXAMPLE 112

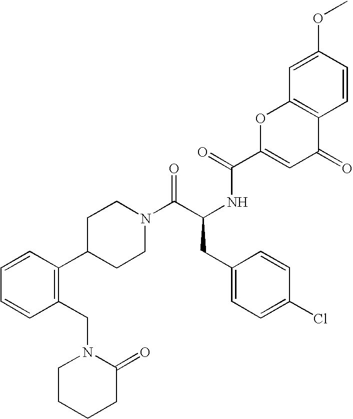

### EXAMPLE 113

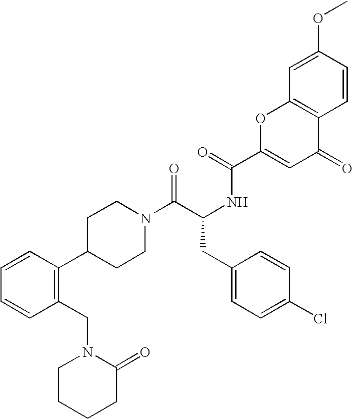

white solid

R_{f}=0.25 (DCM/methanol 95:5).

### EXAMPLE 114

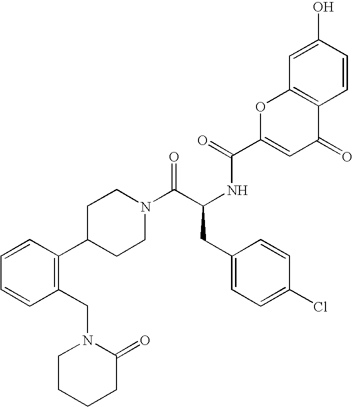

### EXAMPLE 115

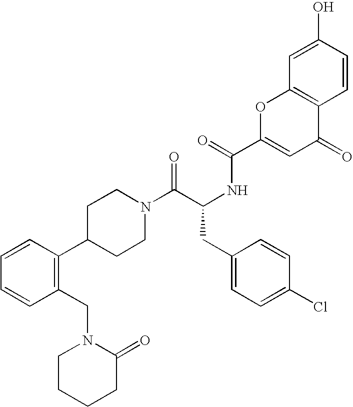

### EXAMPLE 116

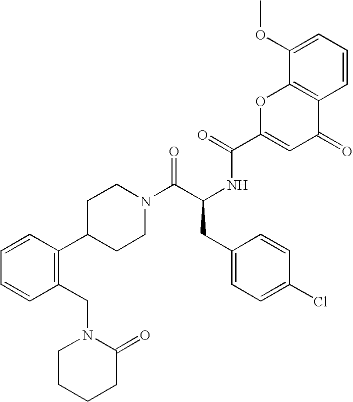

### EXAMPLE 117

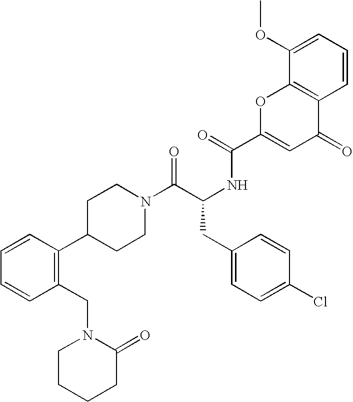

white solid

R_{f}=0.19 (DCM/methanol 95:5).

### EXAMPLE 118

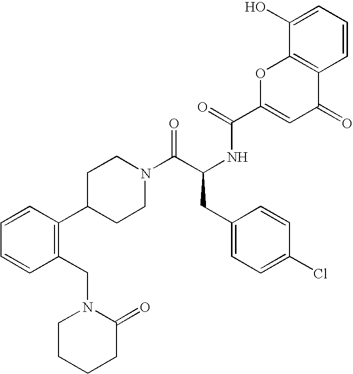

### EXAMPLE 119

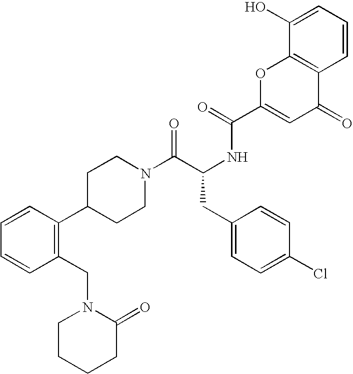

white solid

R_{f}=0.17 (DCM/methanol 95:5).

### EXAMPLE 120

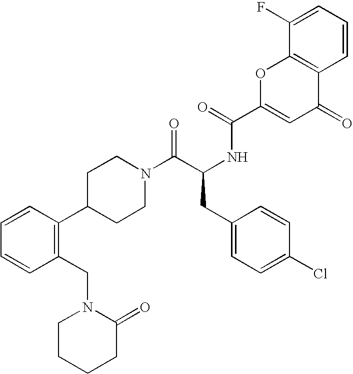

### EXAMPLE 121

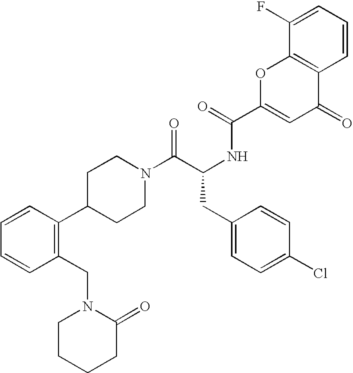

white solid

R_{f}=0.28 (DCM/methanol 95:5).

### EXAMPLE 122

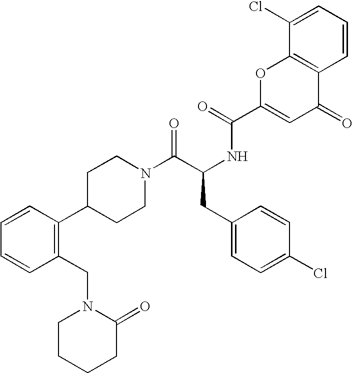

### EXAMPLE 123

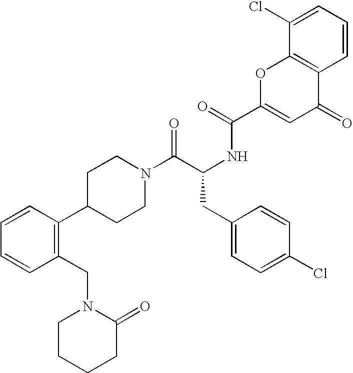

white solid R_{f}=0.30 (DCM/methanol 95:5).

### EXAMPLE 124

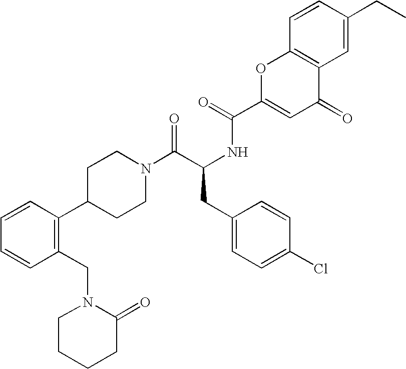

### EXAMPLE 125

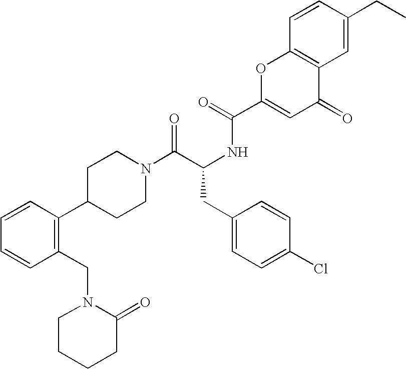

white solid R_{f}=0.36 (DCM/methanol 95:5).

### EXAMPLE 126

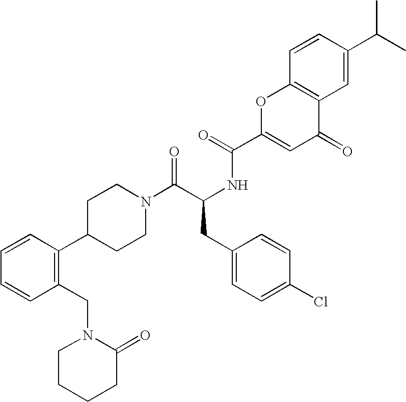

### EXAMPLE 127

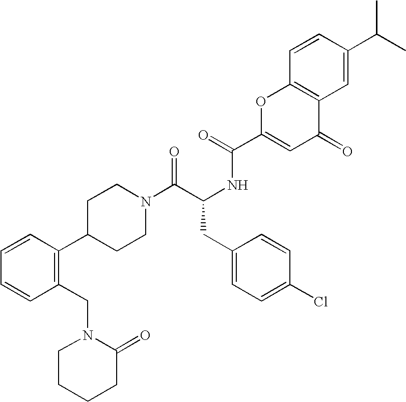

white solid

R_{f}=0.33 (DCM/methanol 95:5).

### EXAMPLE 128

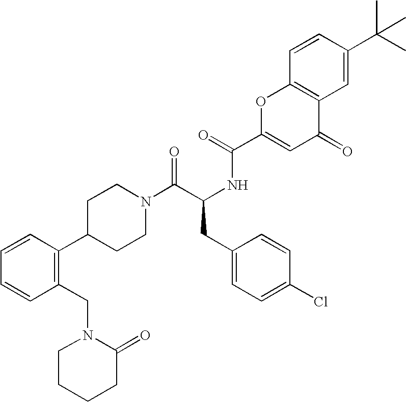

### EXAMPLE 129

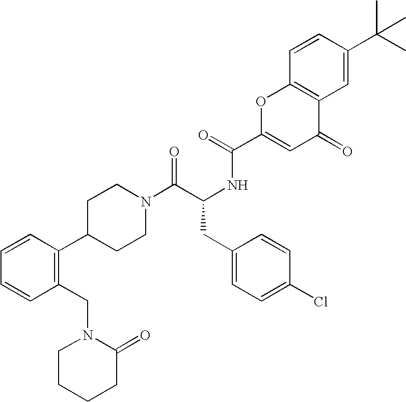

white solid

R_{f}=0.31 (DCM/methanol 95:5).

### EXAMPLE 130

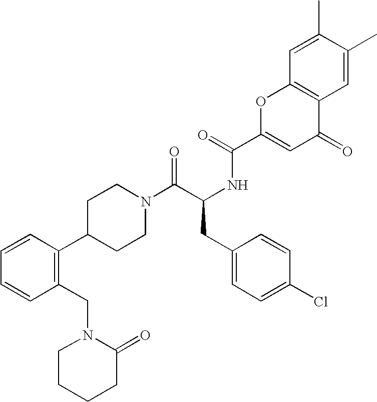

### EXAMPLE 131

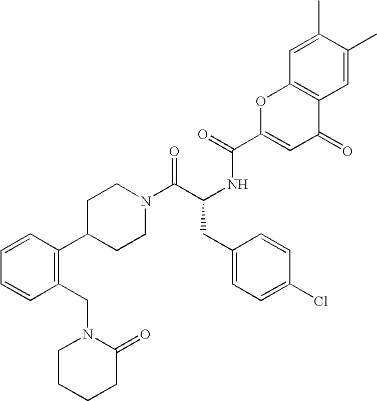

### EXAMPLE 132

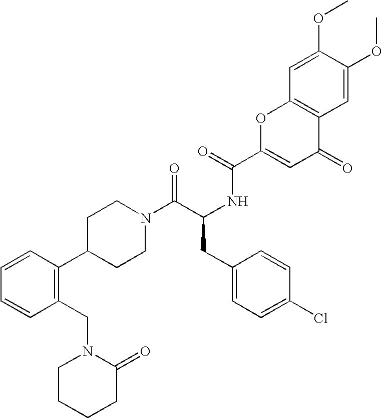

### EXAMPLE 133

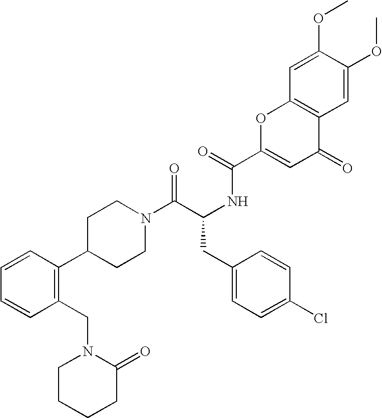

### EXAMPLE 134

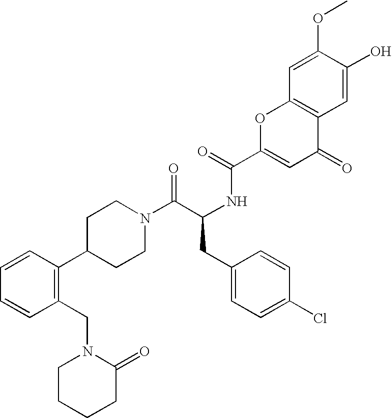

### EXAMPLE 135

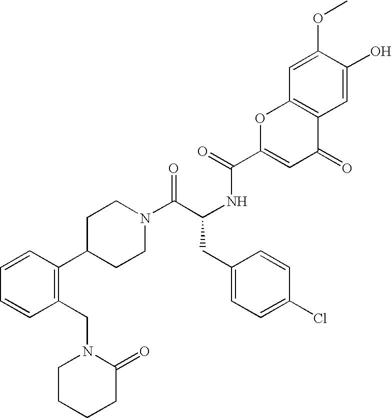

### EXAMPLE 136

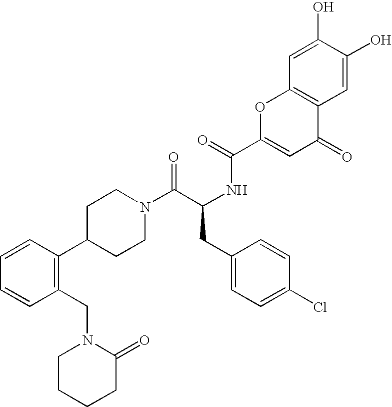

### EXAMPLE 137

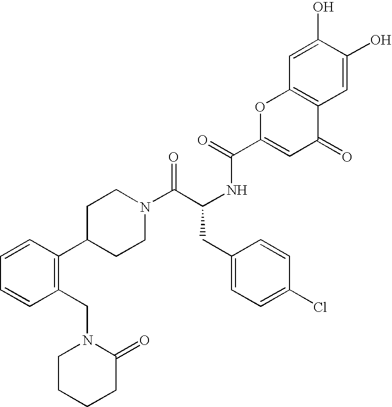

### EXAMPLE 138

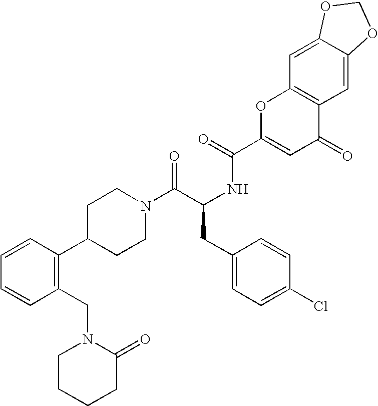

### EXAMPLE 139

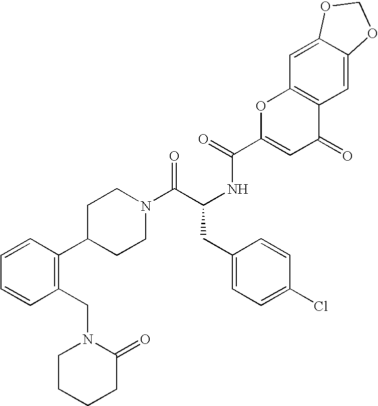

### EXAMPLE 140

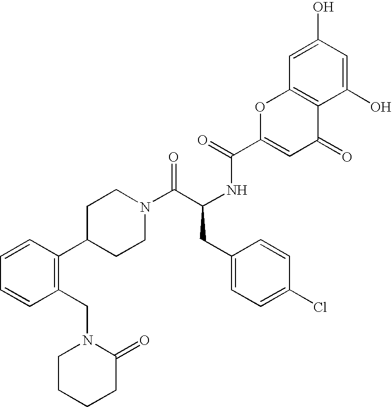

### EXAMPLE 141

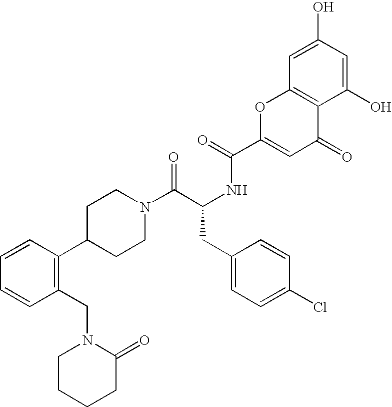

### EXAMPLE 142

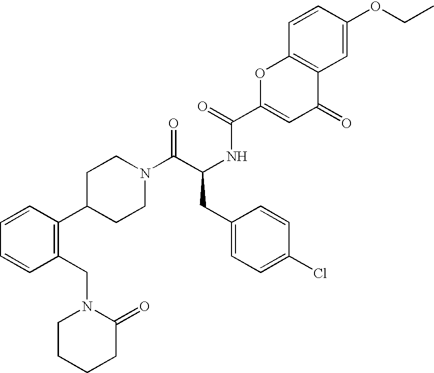

### EXAMPLE 143

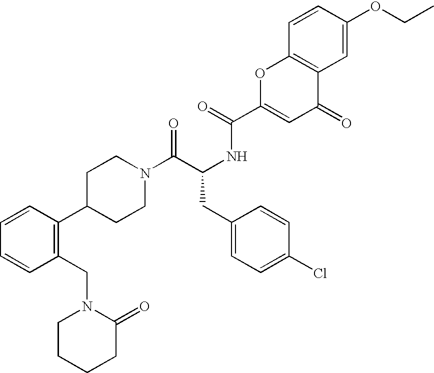

white solid

R_{f}=0.30 (DCM/methanol 95:5).

### EXAMPLE 144

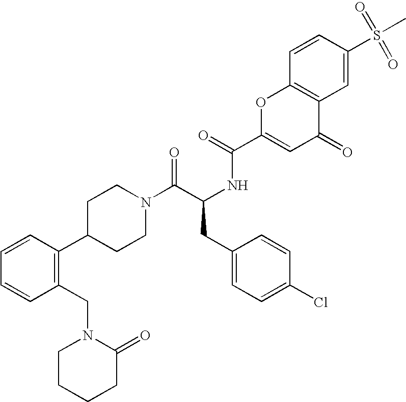

### EXAMPLE 145

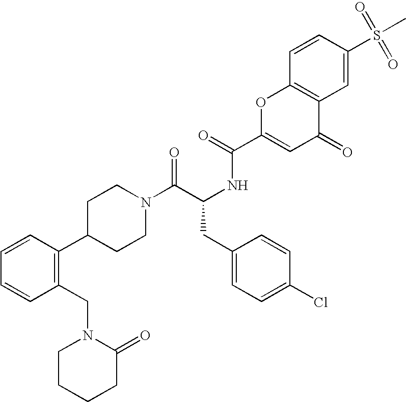

white solid

R_{f}=0.22 (DCM/methanol 95:5).

### EXAMPLE 146

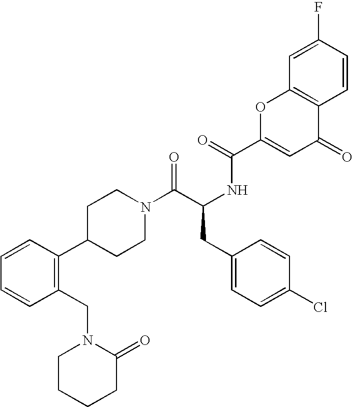

### EXAMPLE 147

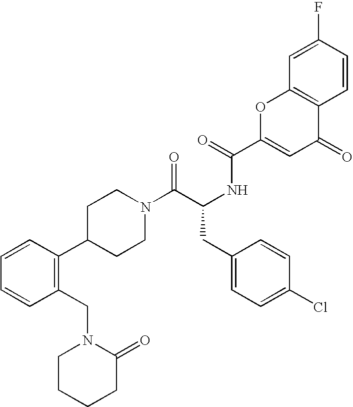

white solid

R_{f}=0.20 (DCM/methanol 95:5).

### EXAMPLE 148

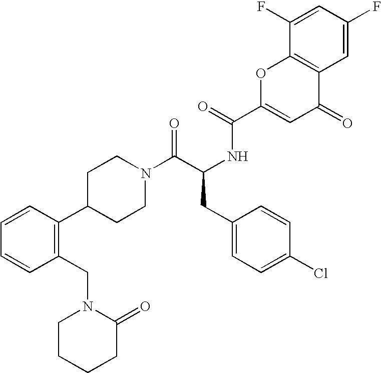

### EXAMPLE 149

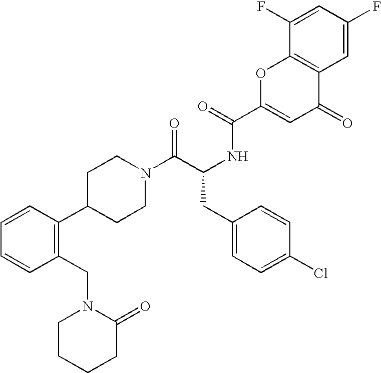

### EXAMPLE 150

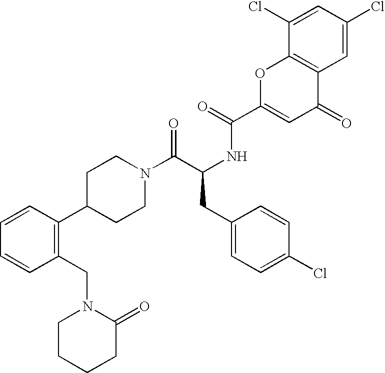

### EXAMPLE 151

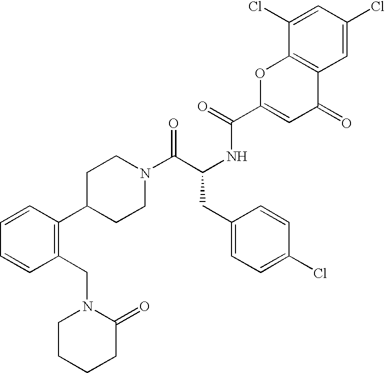

### EXAMPLE 152

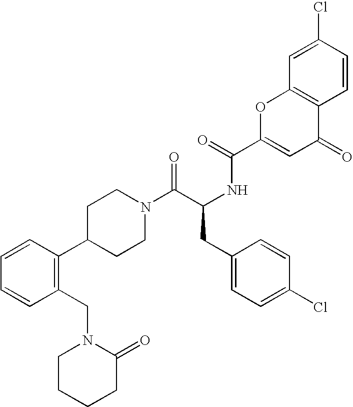

### EXAMPLE 153

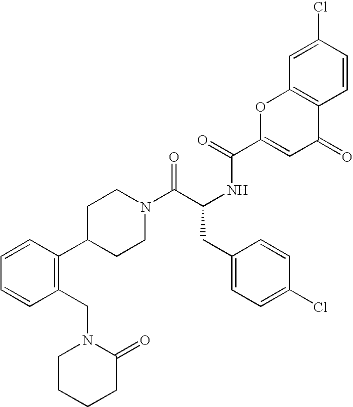

### EXAMPLE 154

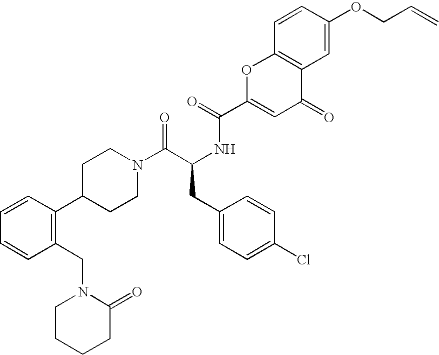

### EXAMPLE 155

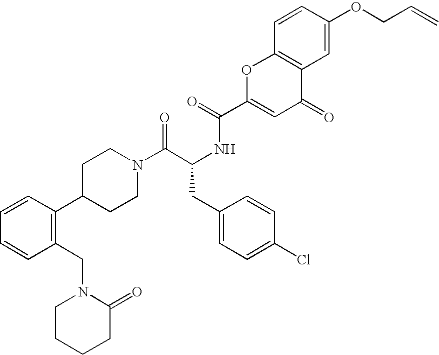

### EXAMPLE 156

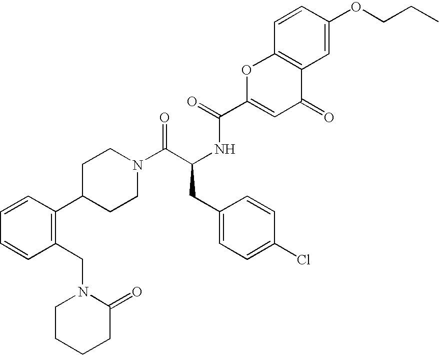

### EXAMPLE 157

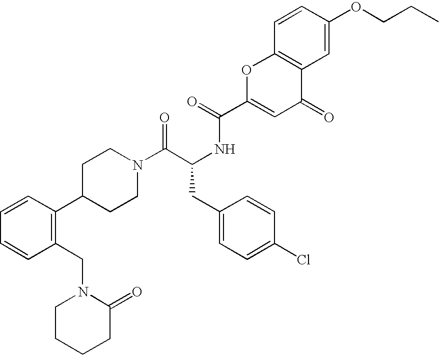

### EXAMPLE 158

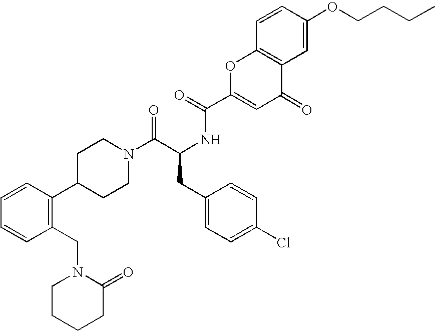

### EXAMPLE 159

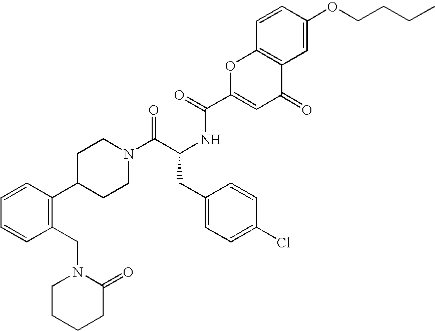

### EXAMPLE 160

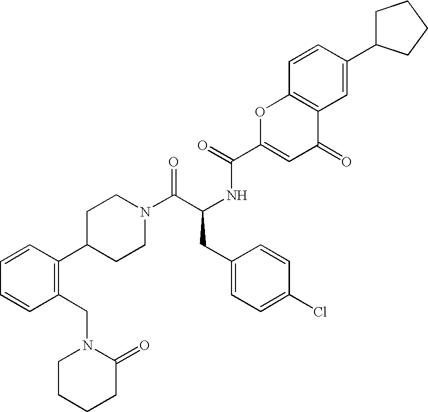

### EXAMPLE 161

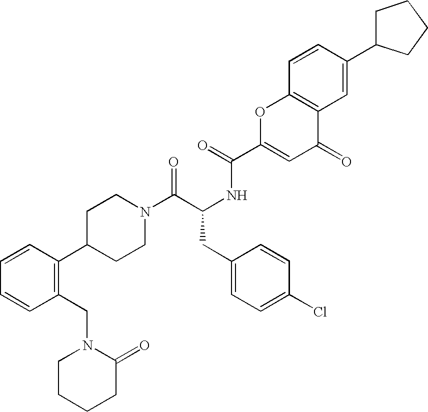

### EXAMPLE 162

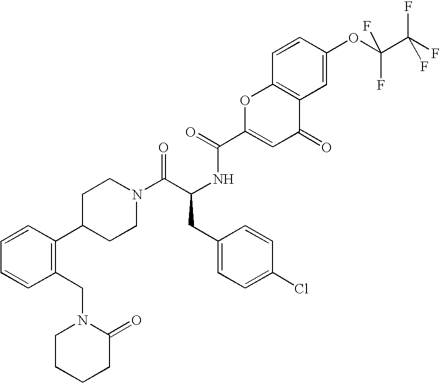

### EXAMPLE 163

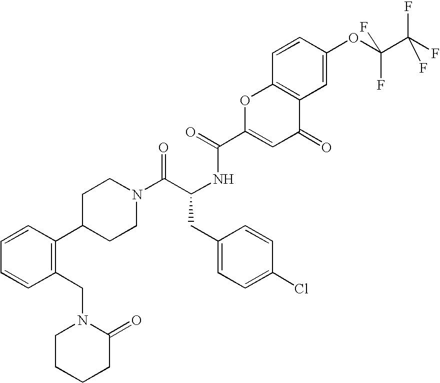

### EXAMPLE 164

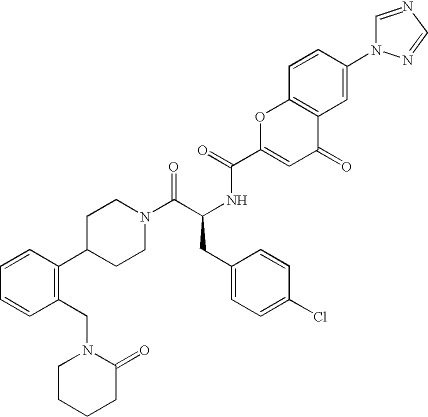

### EXAMPLE 165

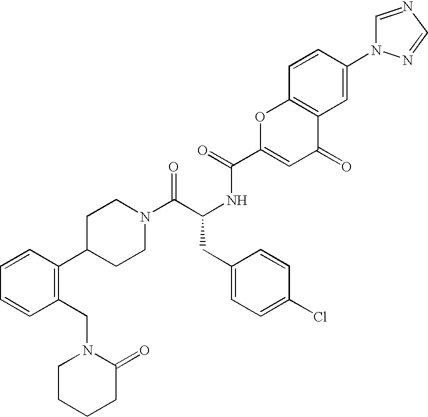

### EXAMPLE 166

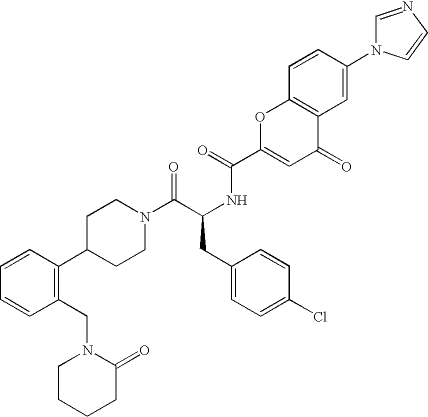

### EXAMPLE 167

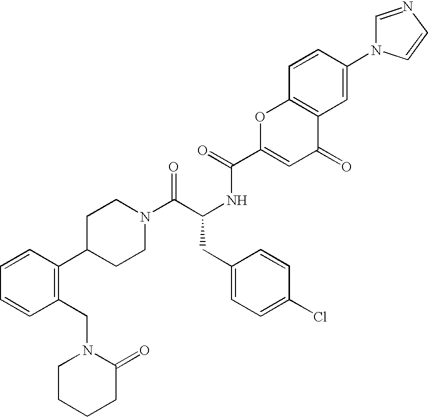

### EXAMPLE 168

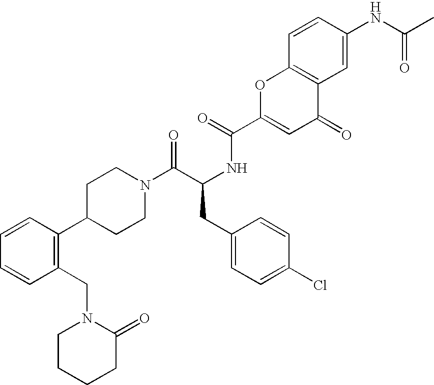

### EXAMPLE 16.9

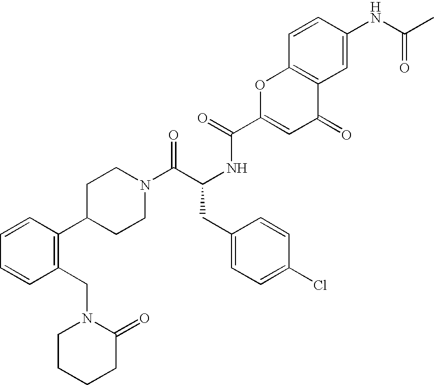

### EXAMPLE 170

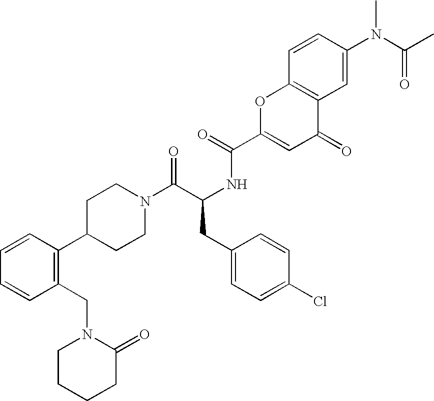

### EXAMPLE 171

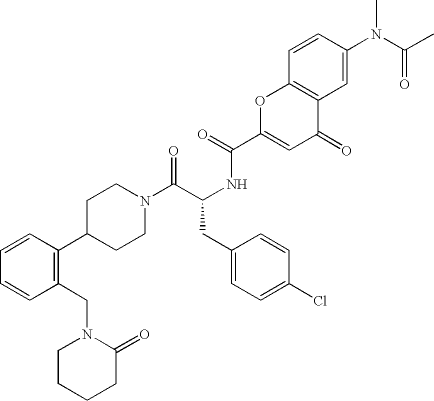

### EXAMPLE 172

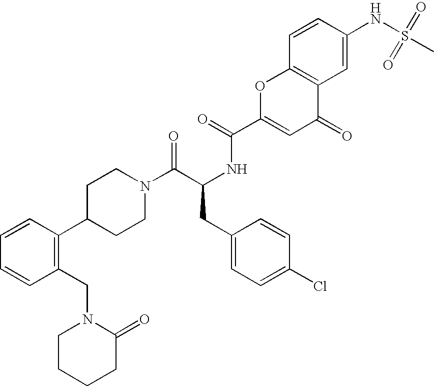

### EXAMPLE 173

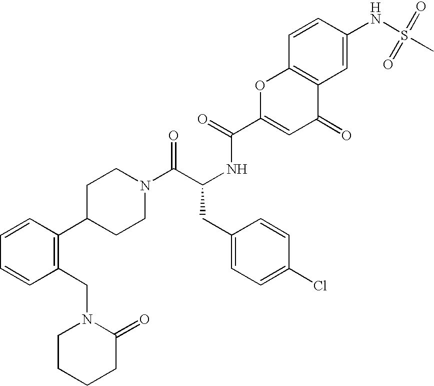

### EXAMPLE 174

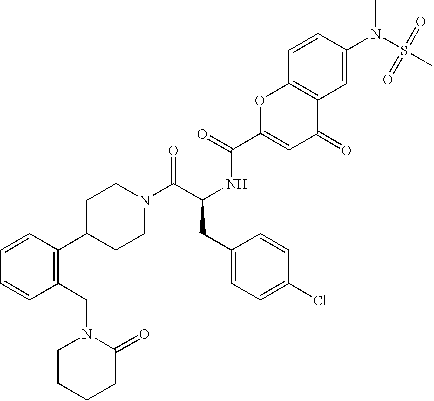

### EXAMPLE 175

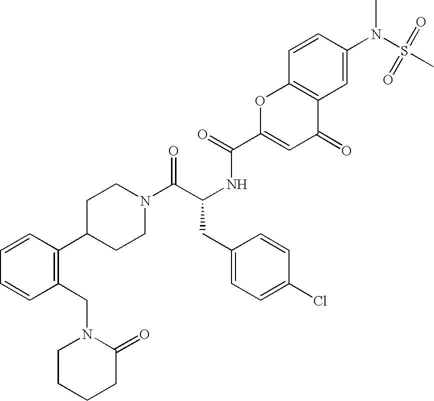

### EXAMPLE 176

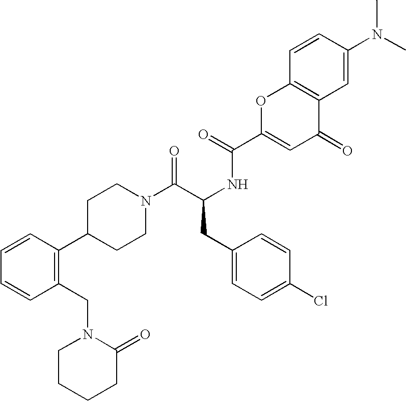

### EXAMPLE 177

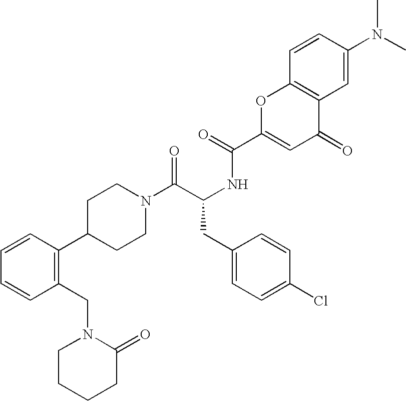

### EXAMPLE 178

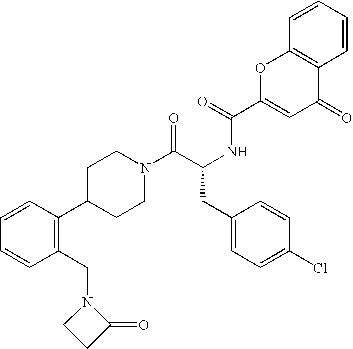

colorless oil

### EXAMPLE 179

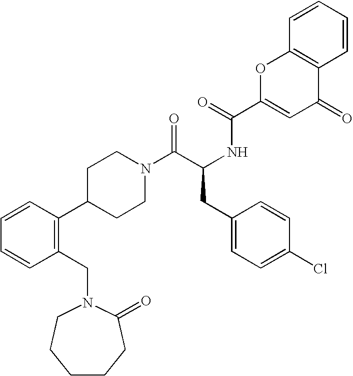

white solid

R_{f}=0.34 (DCM/methanol 95:5).

### EXAMPLE 180

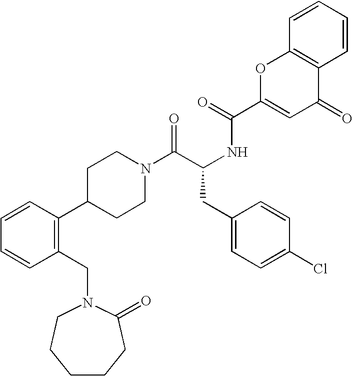

white solid

R_{f}=0.32 (DCM/methanol 95:5).

### EXAMPLE 181

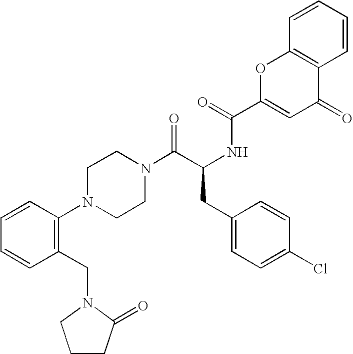

To chromone-2-carboxylic acid (16 mg) in DCM (2 ml) was added intermediate 175d) (36 mg), N-methylmorpholine (14 μl), HOBt (14 mg) and stirred for 20 min. EDC (23 mg) was added and stirring was continued for 1 h. An additional amount of N-methylmorpholine (8 μl) was added and stirred overnight. The reaction mixture was poured into water (5 ml) and the organic phase was separated. The aqueous phase was extracted two times with ethyl acetate. The combined organic phases were washed three times with 0.5 N HCl and three times with saturated sodium bicarbonate solution, dried over Na₂SO₄ and concentrated to yield the product which was purified by column chromatography.

white solid

R_{f}=0.60 (ethyl acetate/ethanol 3:1); Mp. 169-200° C.

The required intermediates can be synthesized in the following way:

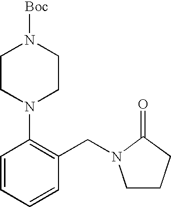

Boc-piperazine (895 mg), intermediate 1a) (1004 mg), Pd₂(dba)₃ (235 mg), BINAP (442 mg) and cesium carbonate (3 g) were mixed together in toluene (20 ml). The mixture was degassed and heated to 100° C. for 3 d. The mixture was diluted with ether (100 ml) and filtered over Celite. The filtrate was concentrated and then subjected to chromatography on silica gel to yield the title compound.

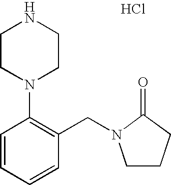

To the Boc-protected amine from 181a) (680 mg) in DCM (10 ml) was added TFA (2 ml) and stirred at room temperature for 90 min. Additional TFA (2 ml) was added and stirred for 10 min. The reaction mixture was diluted with DCM (20 ml) and carefully basified by pouring into 10% aqueous sodium carbonate solution (40 ml). The organic layer was separated and the aqueous layer was further extracted three times with DCM. The combined organics were washed with water and brine, dried over Na₂SO₄, concentrated to give a white solid.

For prolonged storage, the free base was converted into the corresponding hydrochloride. The free base was dissolved in DCM (10 ml) and app. 1 M HCl in ether (20 ml) was added. The precipitate was filtered and the residue was washed three times with ether and dried under reduced pressure to yield the desired product.

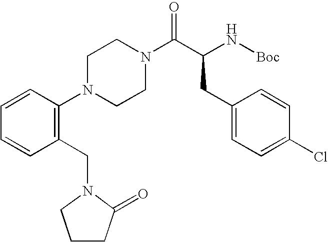

To Boc-L-4-chlorophenylalanine (82 mg) in DCM (5 ml) was added the amine hydrochloride from 1815b) (61 mg), N-methylmorpholine (42 μl), HOBt (48 mg) and stirred for 20 min. EDC (72 mg) was added and stirring was continued for 1 h. An additional amount of N-methylmorpholine (20 μl) was added and stirred overnight. The reaction mixture was poured into water (5 ml) and the organic phase was separated. The aqueous phase was extracted two times with DCM. The combined organic phases were washed with 0.5 N HCl and saturated sodium bicarbonate solution, dried over Na₂SO₄ and concentrated to yield the title compound which was purified by column chromatography.

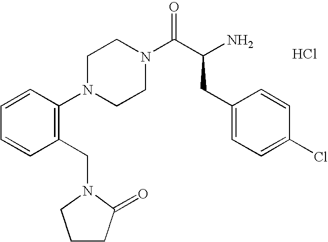

To the Boc-protected amine from 181c) (78 mg) in DCM (5 ml) was added TFA (1 ml) and stirred at room temperature for 90 min. Additional TFA (1 ml) was added and stirred for 10 min. The reaction mixture was diluted with DCM (10 ml) and carefully basified by pouring into 10% aqueous sodium carbonate solution (20 ml). The organic layer was separated and the aqueous layer was further extracted three times with DCM. The combined organics were washed with water and brine, dried over Na₂SO₄, concentrated to give a white solid.

For prolonged storage, the free base was converted into the corresponding hydrochloride. The free base was dissolved in DCM (5 ml) and app. 1 M HCl in ether (10 ml), was added. The precipitate was filtered and the residue was washed three times with ether and dried under reduced pressure to yield the desired product.

The following examples can be prepared in a similar way:

### EXAMPLE 182

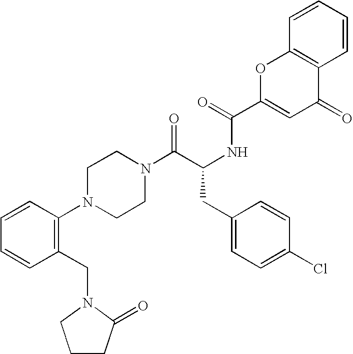

white solid

R_{f}=0.70 (ethyl acetate); Mp. 171-182° C.

### EXAMPLE 183

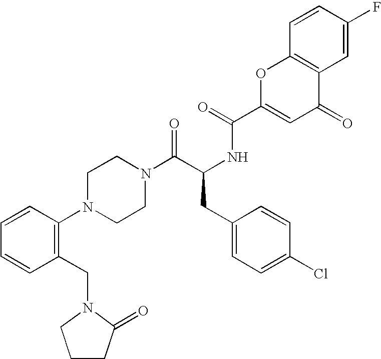

### EXAMPLE 184

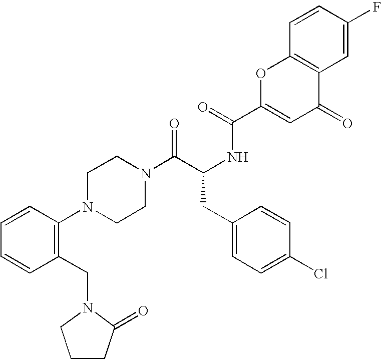

white solid

R_{f}=0.70 (ethyl acetate); Mp. 125-130° C.

### EXAMPLE 185

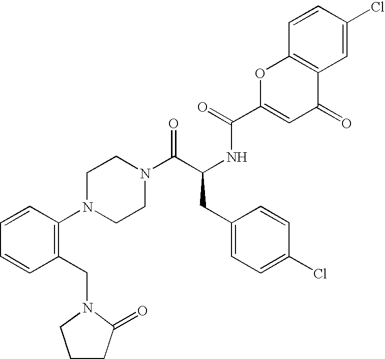

### EXAMPLE 186

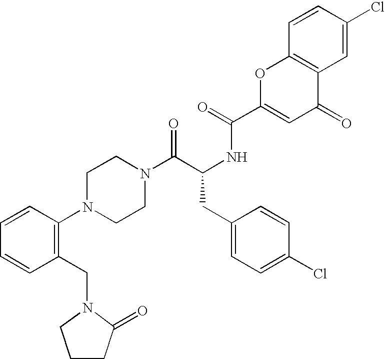

white solid

R_{f}=0.68 (ethyl acetate); Mp. 126-134° C.

### EXAMPLE 187

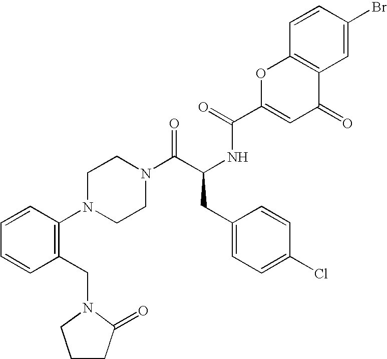

### EXAMPLE 188

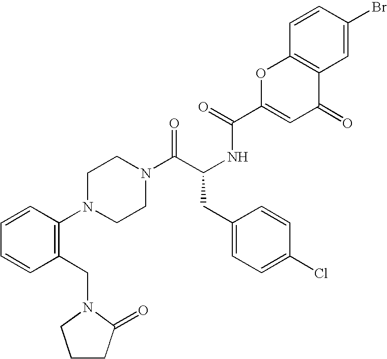

white solid

R_{f}=0.68 (ethyl acetate); Mp. 125-136° C.

### EXAMPLE 189

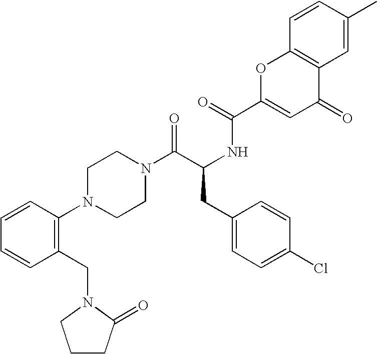

### EXAMPLE 190

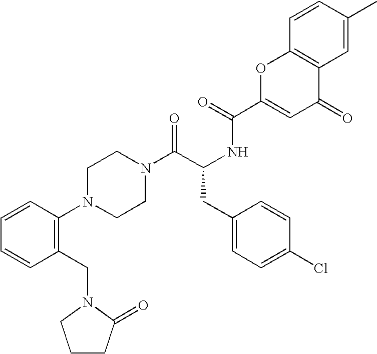

white solid

R_{f}=0.64 (ethyl acetate); Mp. 119-129° C.

### EXAMPLE 191

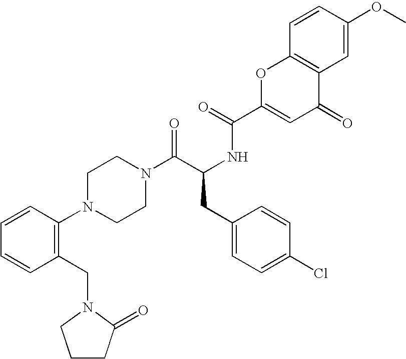

### EXAMPLE 192

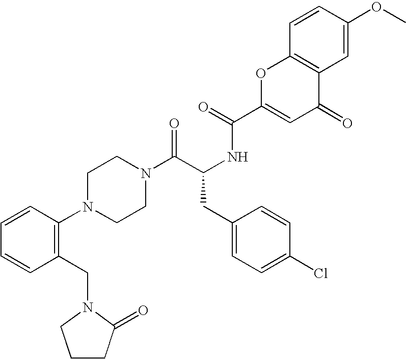

white solid

R_{f}=0.10 (ethyl acetate); Mp. 150-155° C.

### EXAMPLE 193

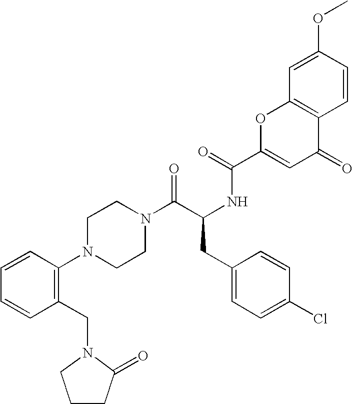

### EXAMPLE 194

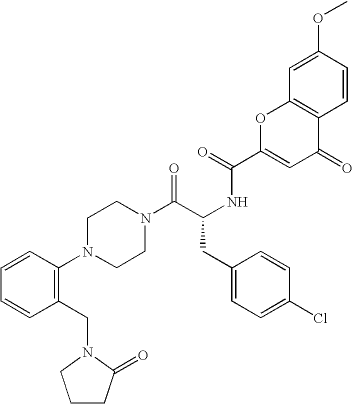

white solid

R_{f}=0.58 (ethyl acetate/ethanol 3:1); Mp. 112-121° C.

### EXAMPLE 195

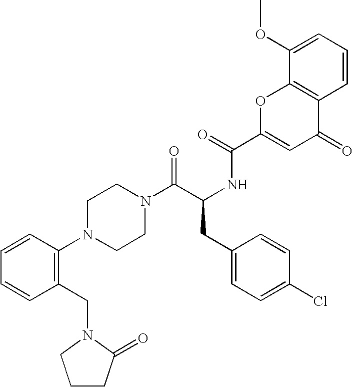

### EXAMPLE 196

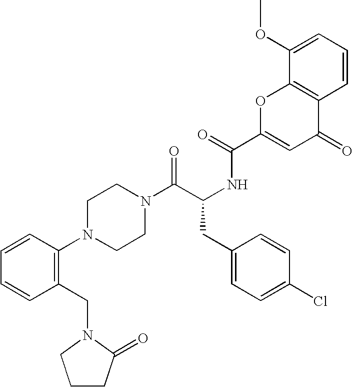

white solid

R_{f}=0.52 (ethyl acetate/ethanol 3:1); Mp. 116-124° C.

### EXAMPLE 197

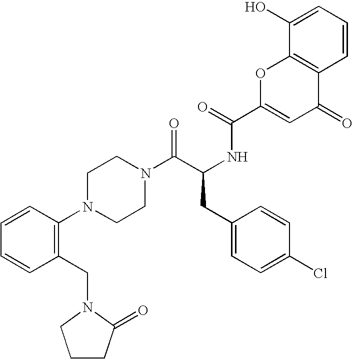

### EXAMPLE 198

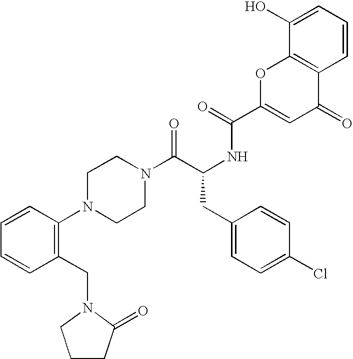

white solid

R_{f}=0.61 (ethyl acetate/ethanol 3:1); Mp. 151-1160° C.

### EXAMPLE 199

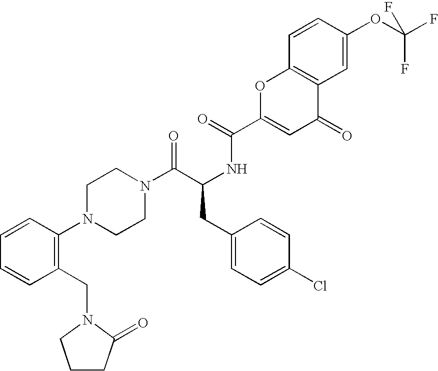

### EXAMPLE 200

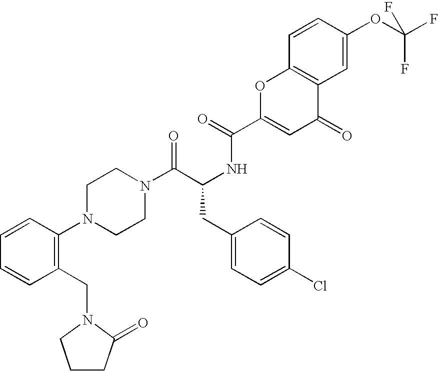

white solid

R_{f}=0.65 (ethyl acetate/ethanol 3:1); Mp. 111-119° C.

### EXAMPLE 201

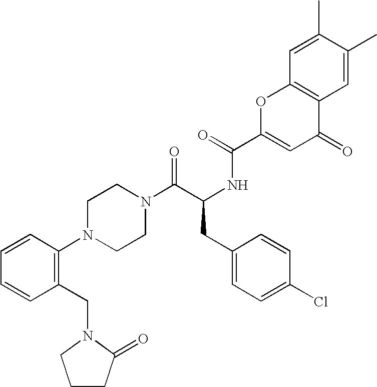

### EXAMPLE 202

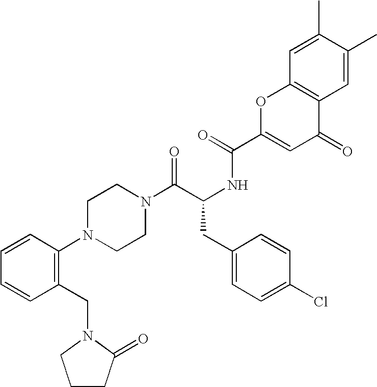

white solid

R_{f}=0.61 (ethyl acetate/ethanol 3:1); Mp. 125-134° C.

### EXAMPLE 203

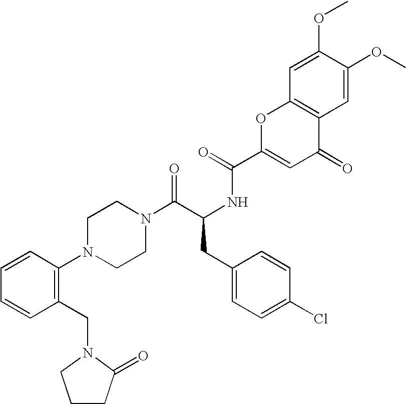

### EXAMPLE 204

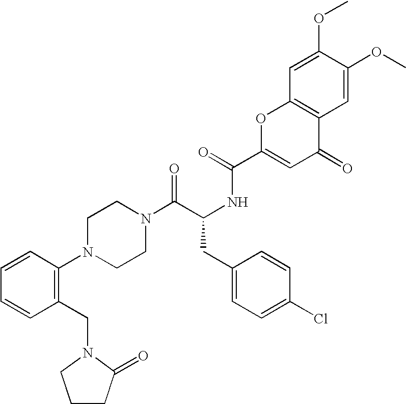

white solid

R_{f}=0.51 (ethyl acetate/ethanol 3:1); Mp. 135-144° C.

### EXAMPLE 205

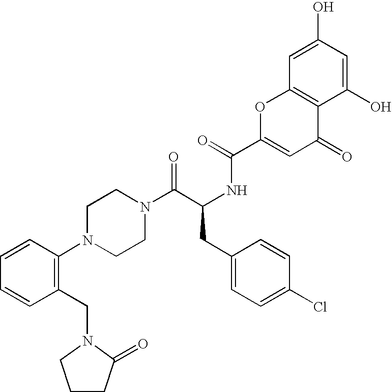

### EXAMPLE 206

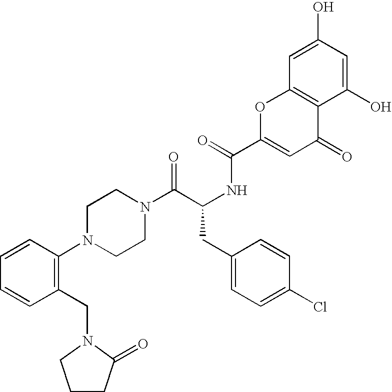

beige sold

R_{f}=0.66 (ethyl acetate/ethanol 3:1); Mp. 156-165° C.

### EXAMPLE 207

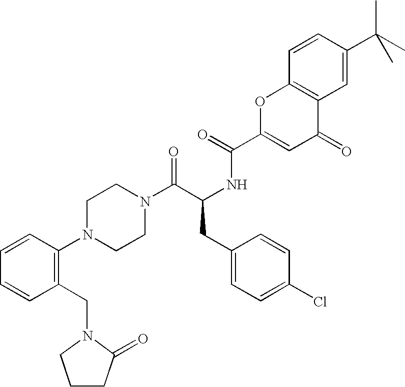

### EXAMPLE 208

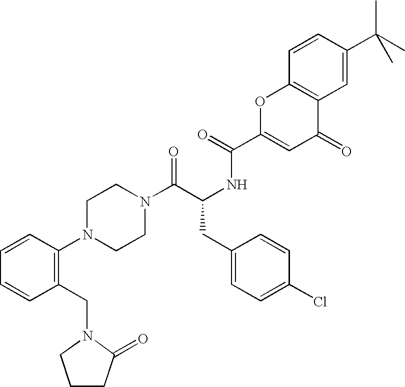

white solid

R_{f}=0.68 (ethyl acetate/ethanol 3:1); Mp. 129-141° C.

### EXAMPLE 209

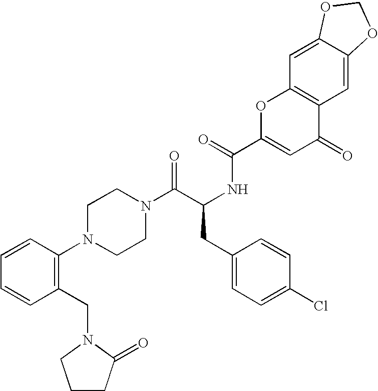

### EXAMPLE 210

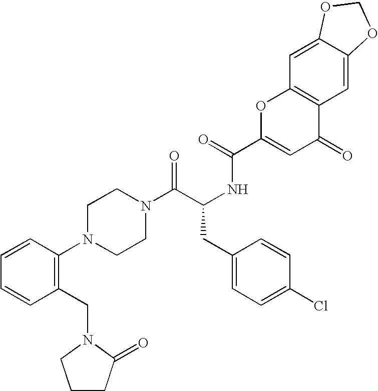

white solid

R_{f}=0.63 (ethyl acetate/ethanol 3:1); Mp. 148-152° C.

### EXAMPLE 211

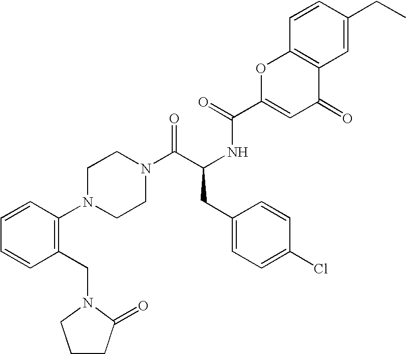

### EXAMPLE 212

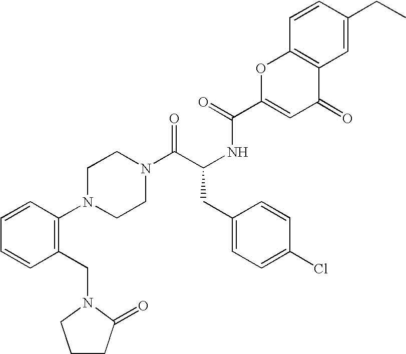

white solid

R_{f}=0.63 (ethyl acetate/ethanol 3:1); Mp. 1119-124° C.

### EXAMPLE 213

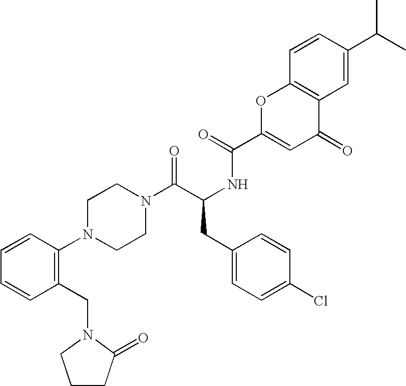

### EXAMPLE 214

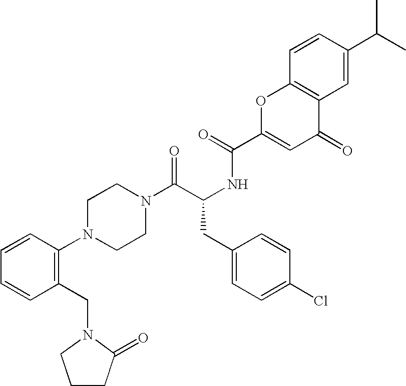

white solid

R_{f}=0.66 (ethyl acetate/ethanol 3:1); Mp. 120-126° C.

### EXAMPLE 215

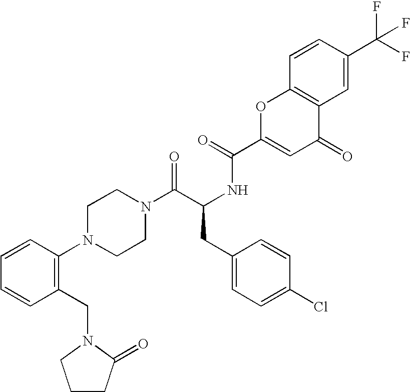

### EXAMPLE 216

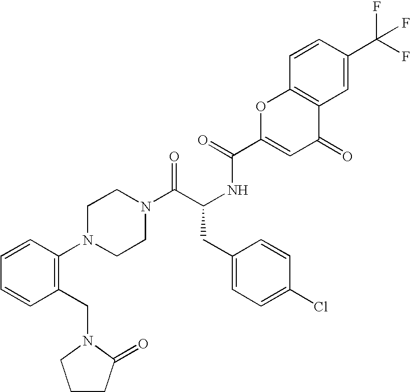

### EXAMPLE 217

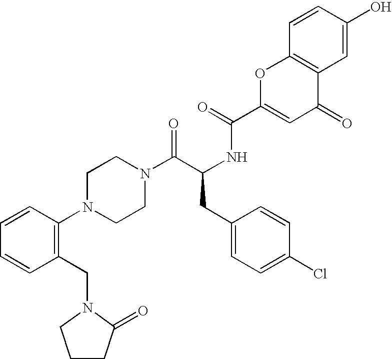

### EXAMPLE 218

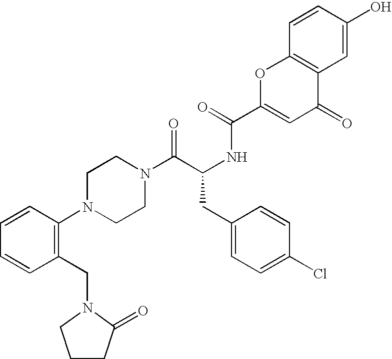

### EXAMPLE 219

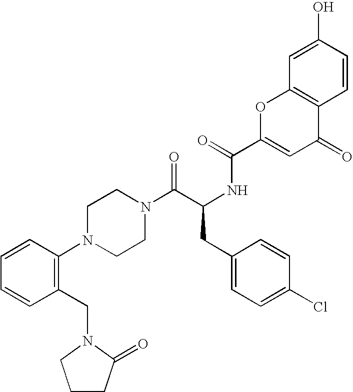

### EXAMPLE 220

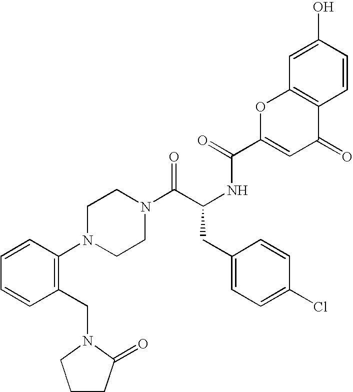

### EXAMPLE 221

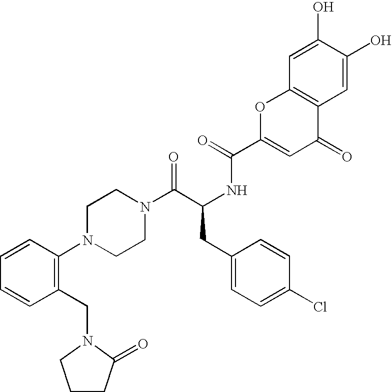

### EXAMPLE 222

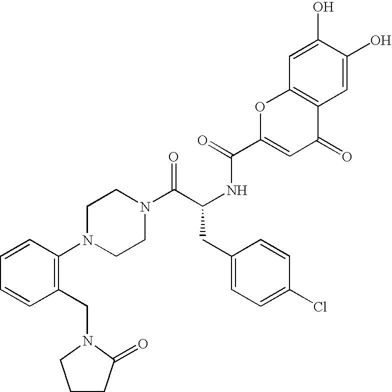

### EXAMPLE 223

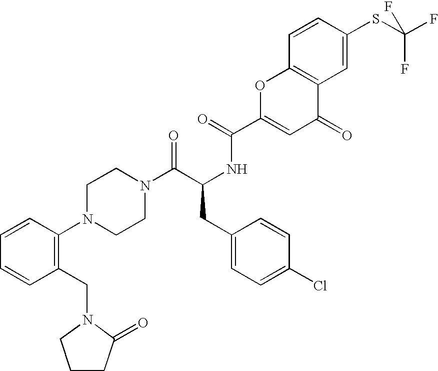

### EXAMPLE 224

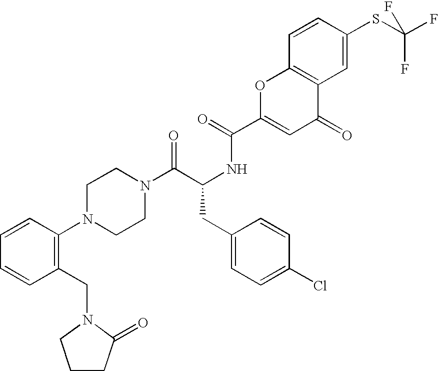

white solid

R_{f}=0.67 (ethyl acetate/ethanol); Mp. 101-105° C.

### EXAMPLE 225

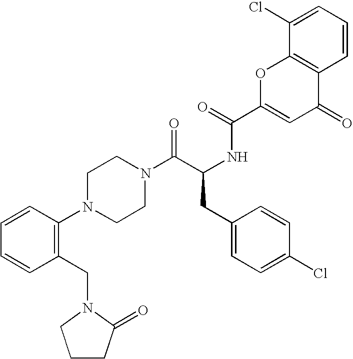

### EXAMPLE 226

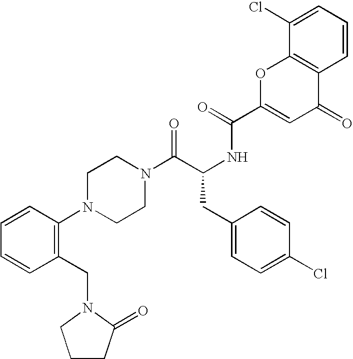

white solid

R_{f}=0.67 (ethyl acetate/ethanol 3:1); Mp. 121-125° C.

### EXAMPLE 227

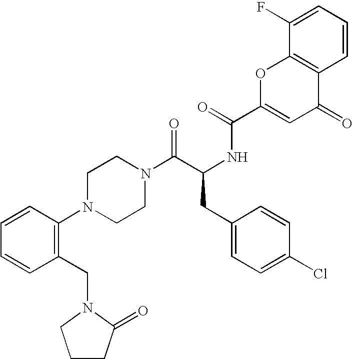

### EXAMPLE 228

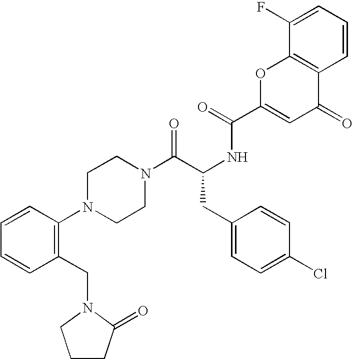

white solid

R_{f}=0.63 (ethyl acetate/ethanol 3:1); Mp. 124-127° C.

### EXAMPLE 229

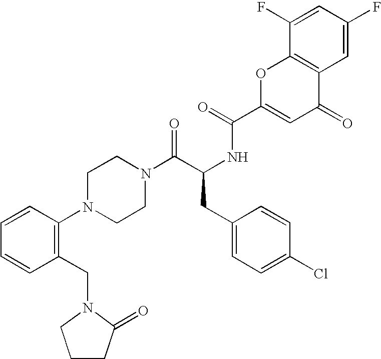

### EXAMPLE 230

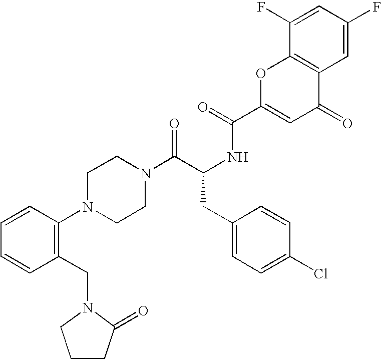

### EXAMPLE 231

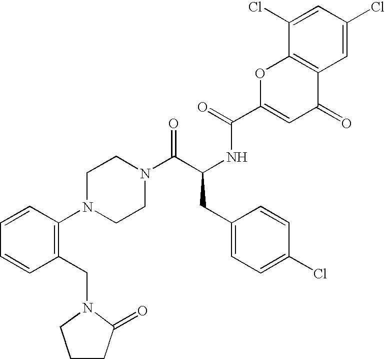

### EXAMPLE 232

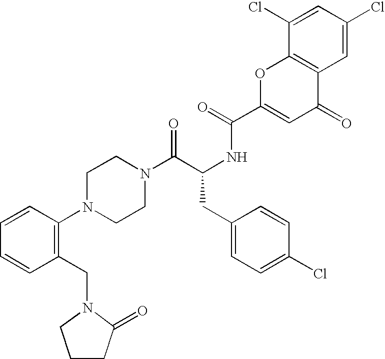

### EXAMPLE 233

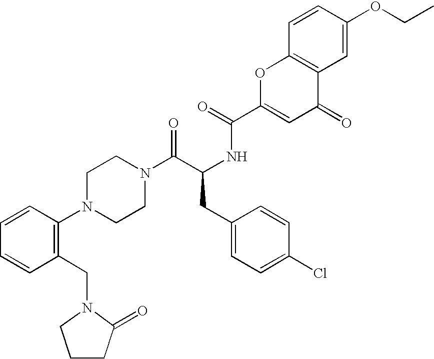

### EXAMPLE 234

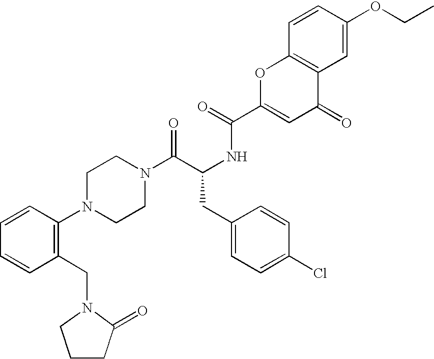

### EXAMPLE 235

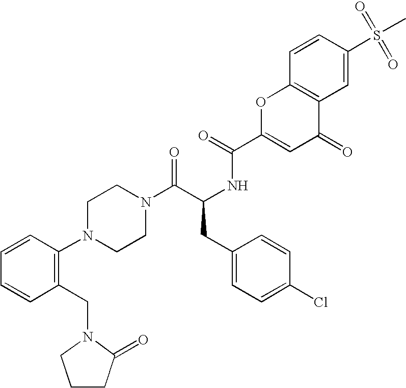

### EXAMPLE 236

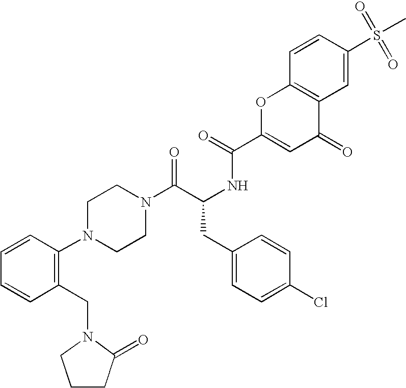

### EXAMPLE 237

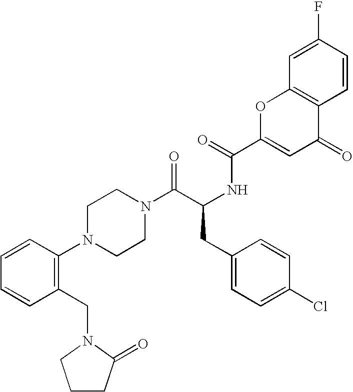

### EXAMPLE 238

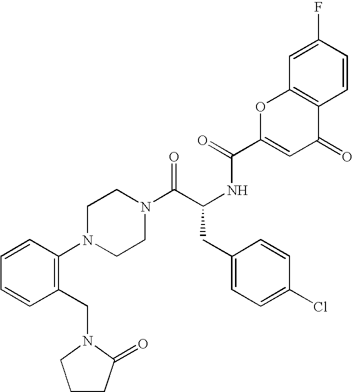

### EXAMPLE 239

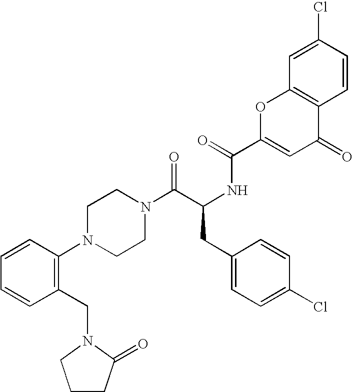

### EXAMPLE 240

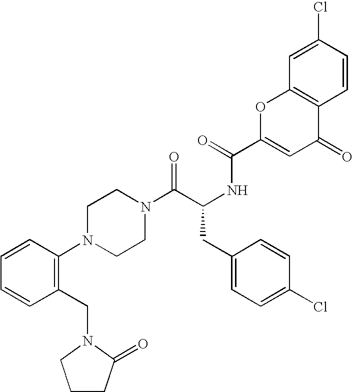

### EXAMPLE 241

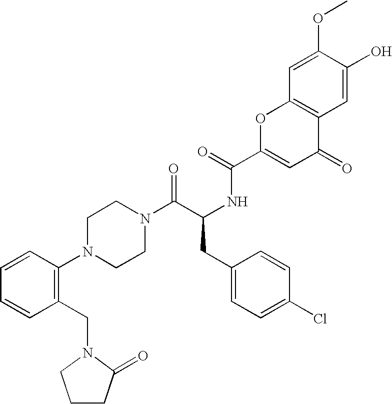

### EXAMPLE 242

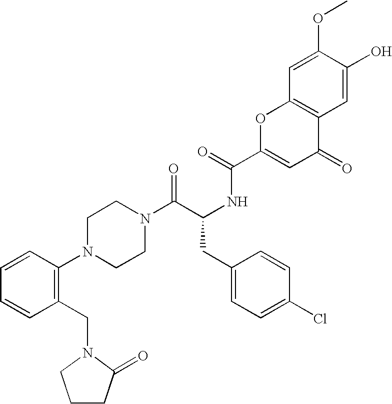

### EXAMPLE 243

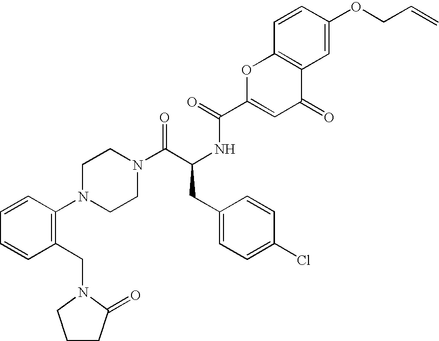

### EXAMPLE 244

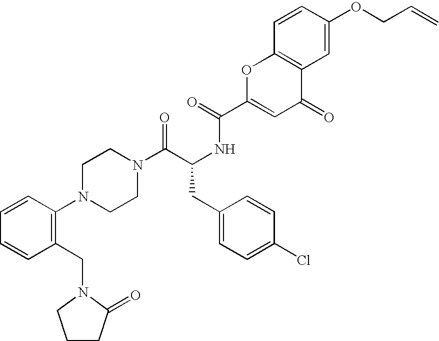

### EXAMPLE 245

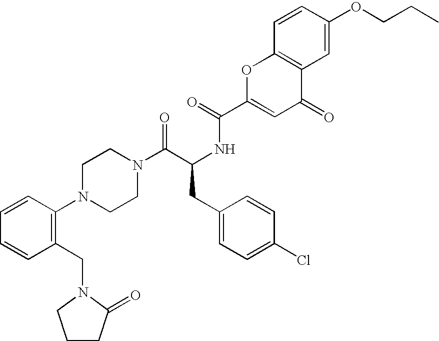

### EXAMPLE 246

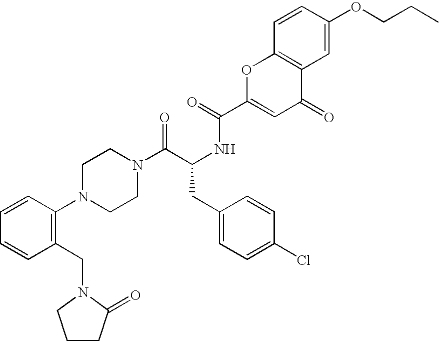

### EXAMPLE 247

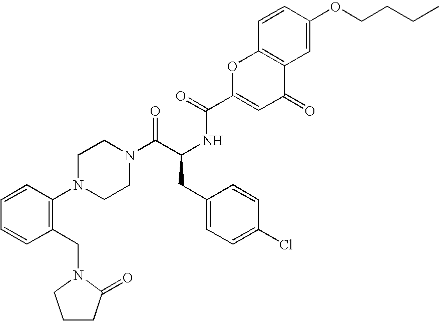

### EXAMPLE 248

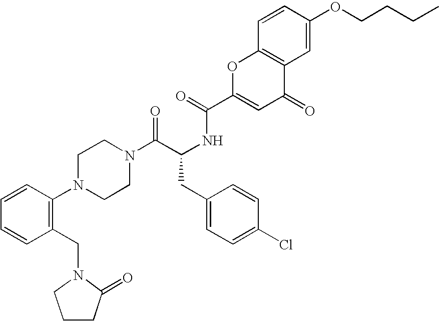

### EXAMPLE 249

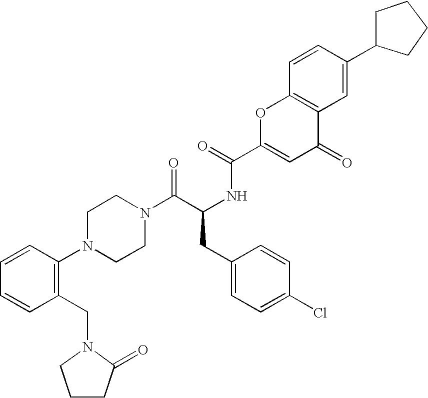

### EXAMPLE 250

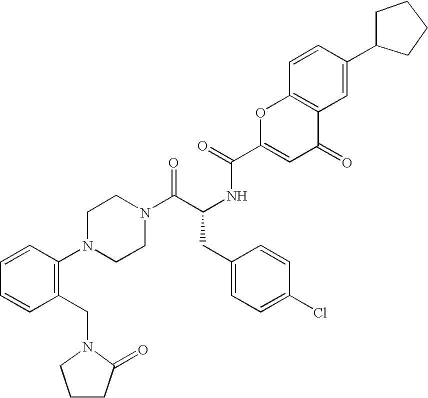

### EXAMPLE 251

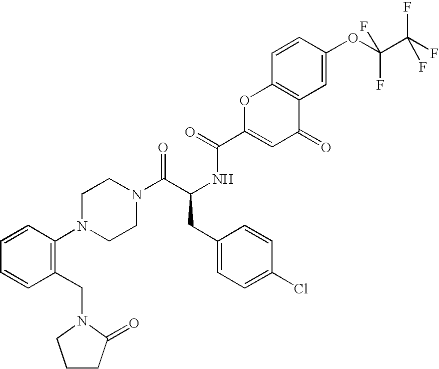

### EXAMPLE 252

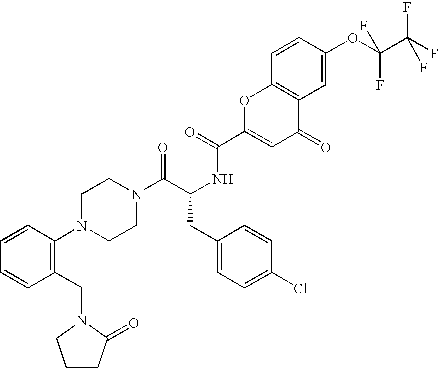

### EXAMPLE 253

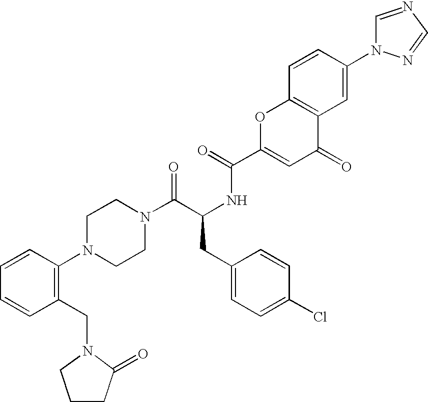

### EXAMPLE 254

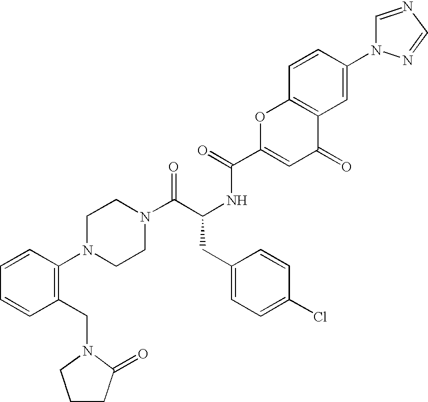

### EXAMPLE 255

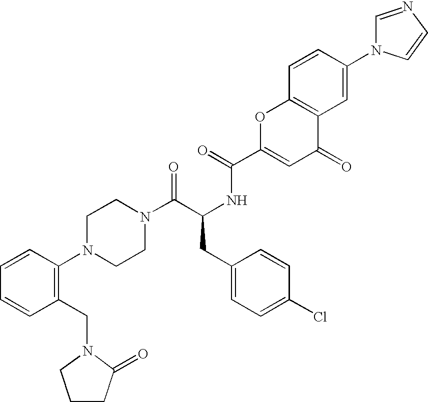

### EXAMPLE 256

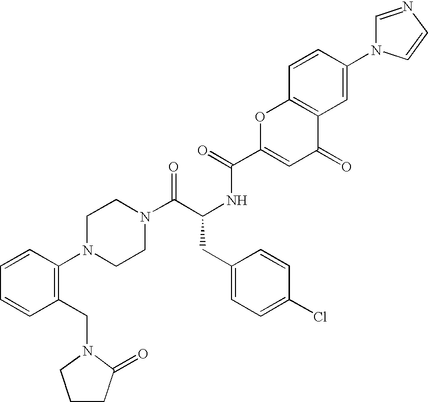

### EXAMPLE 257

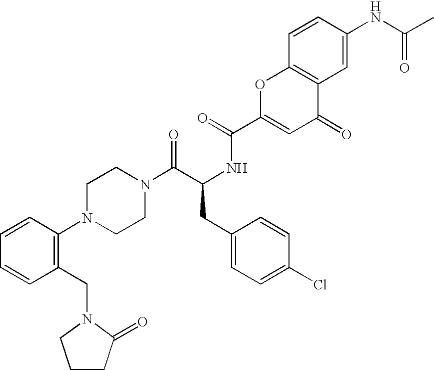

### EXAMPLE 258

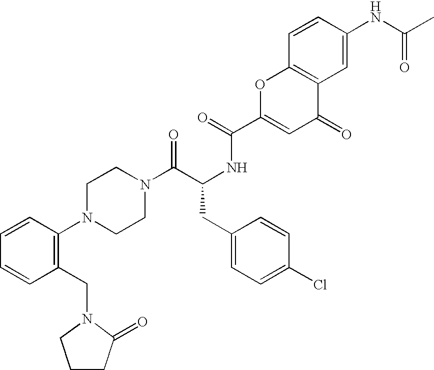

### EXAMPLE 259

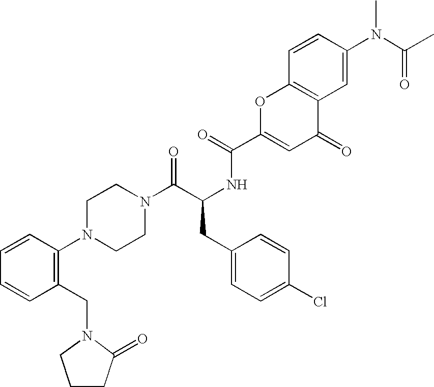

### EXAMPLE 260

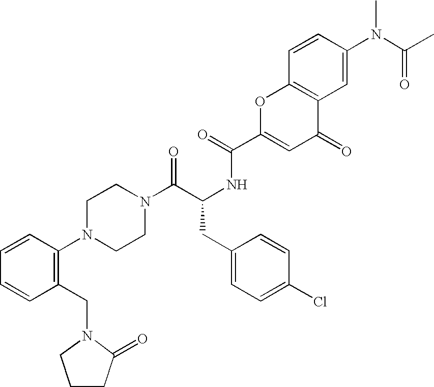

### EXAMPLE 261

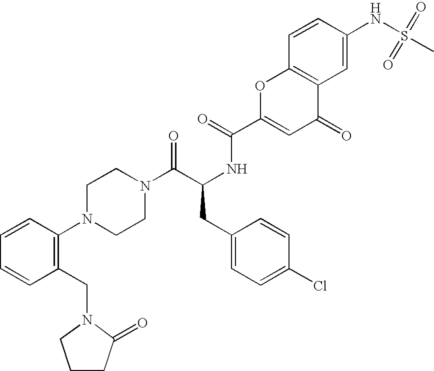

### EXAMPLE 262

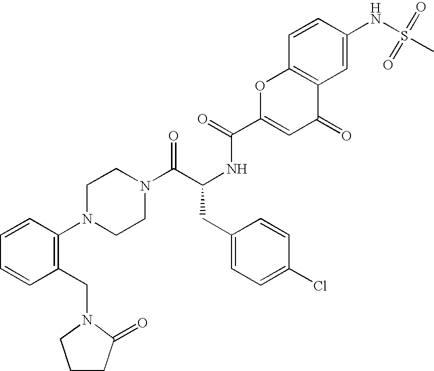

### EXAMPLE 263

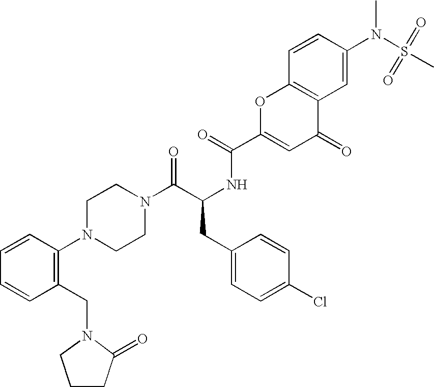

### EXAMPLE 264

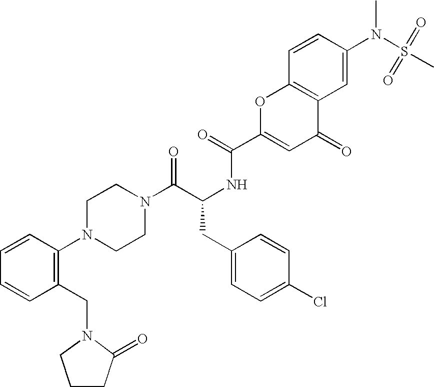

### EXAMPLE 265

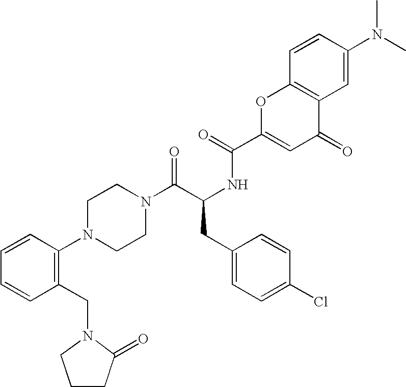

### EXAMPLE 266

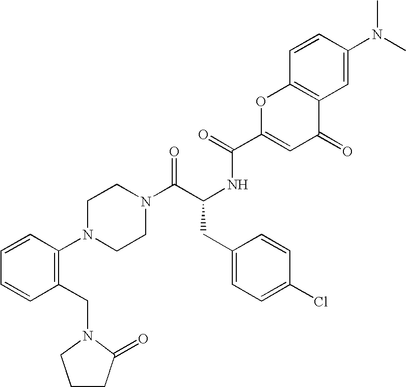

### EXAMPLE 267

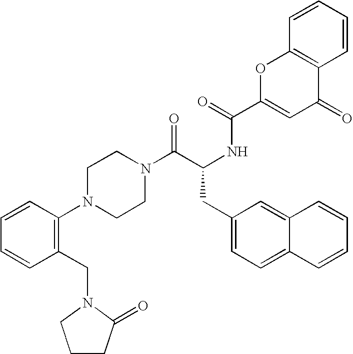

white solid

R_{f}=0.72 (ethyl acetate/ethanol 3:1); Mp. 127-144° C.

### EXAMPLE 268

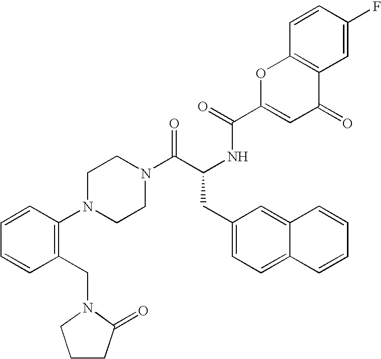

white solid

R_{f}=0.71 (ethyl acetate/ethanol 3:1); Mp. 140-149° C.

### EXAMPLE 269

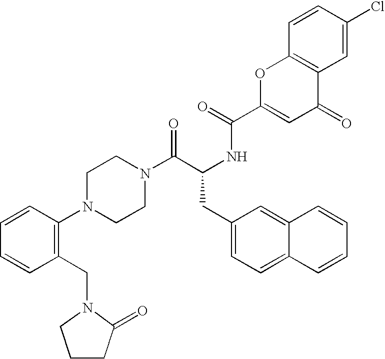 pale yellow solid

R_{f}=0.76 (ethyl acetate/ethanol 3:1); Mp. 137-145° C.

### EXAMPLE 270

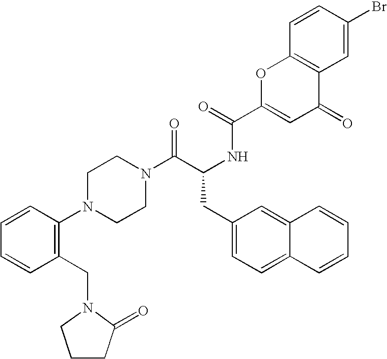

pale yellow solid R_{f}=0.71 (ethyl acetate/ethanol 3:1); Mp. 138-147° C.

### EXAMPLE 271

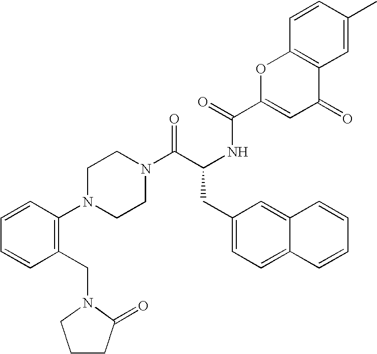

white solid

R_{f}=0.69 (ethyl acetate/ethanol 3:1); Mp. 138-144° C.

### EXAMPLE 272

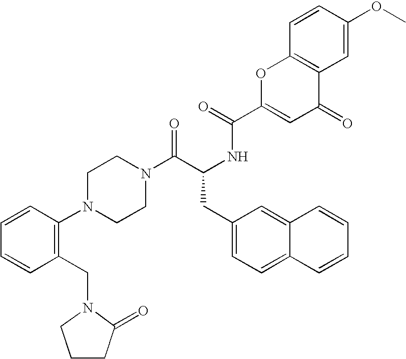

### EXAMPLE 273

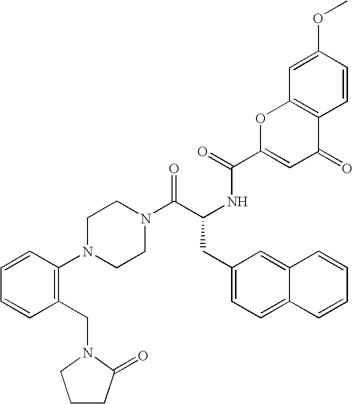

### EXAMPLE 274

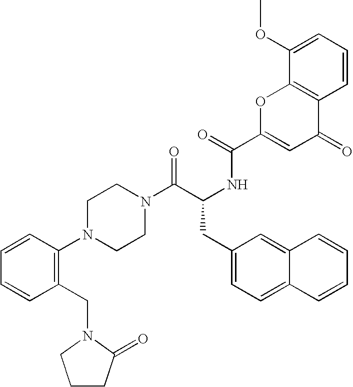

### EXAMPLE 275

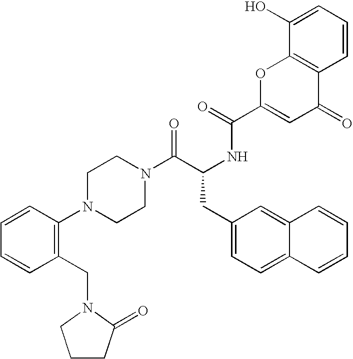

### EXAMPLE 276

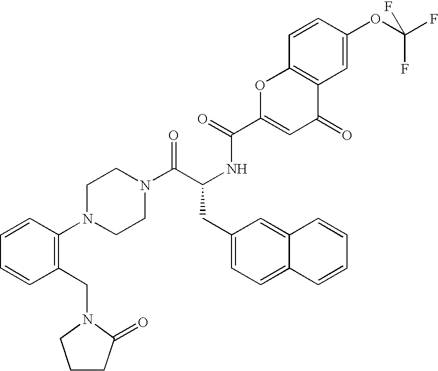

### EXAMPLE 277

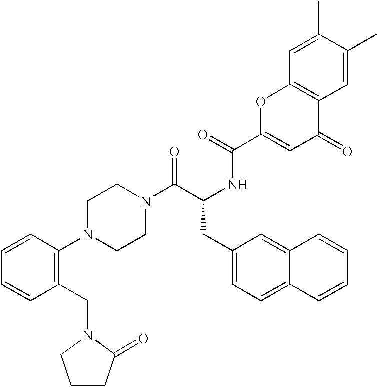

### EXAMPLE 278

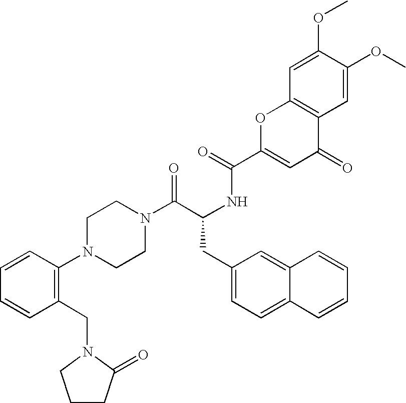

### EXAMPLE 279

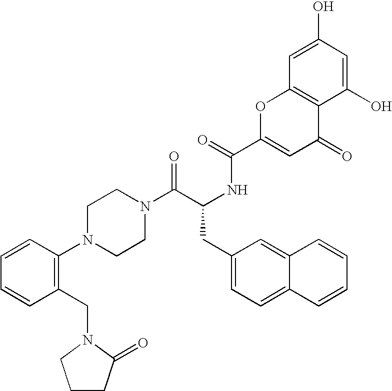

### EXAMPLE 280

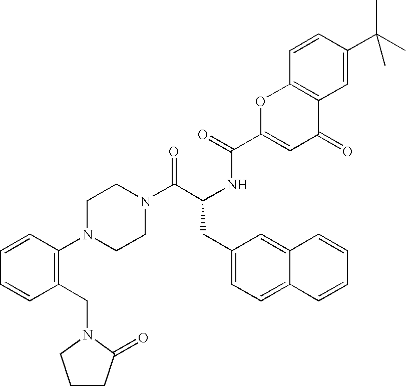

### EXAMPLE 281

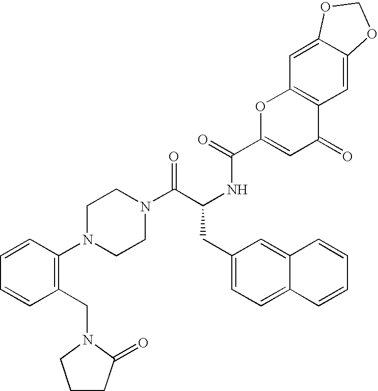

### EXAMPLE 282

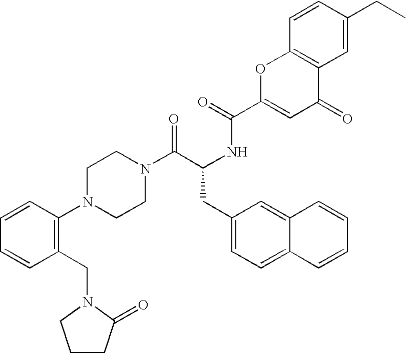

### EXAMPLE 283

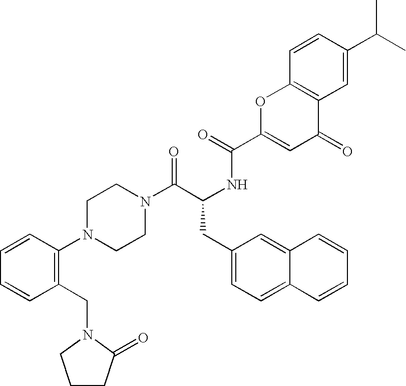

### EXAMPLE 284

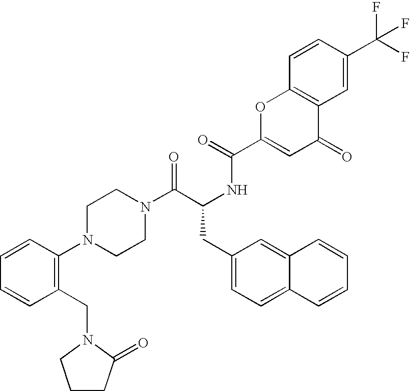

### EXAMPLE 285

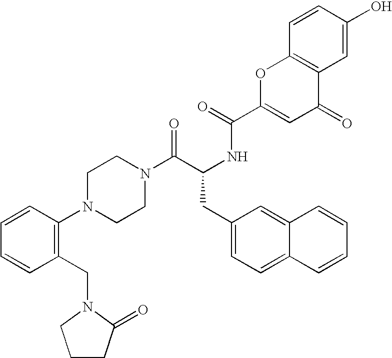

### EXAMPLE 286

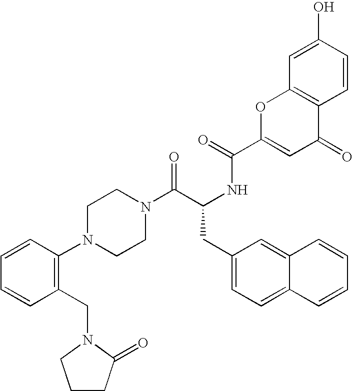

### EXAMPLE 287

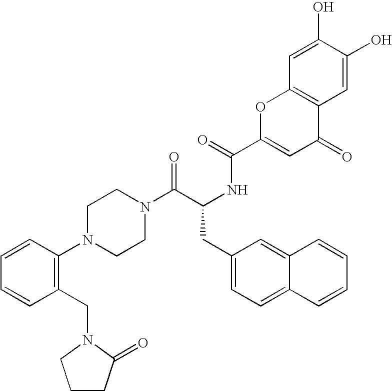

### EXAMPLE 288

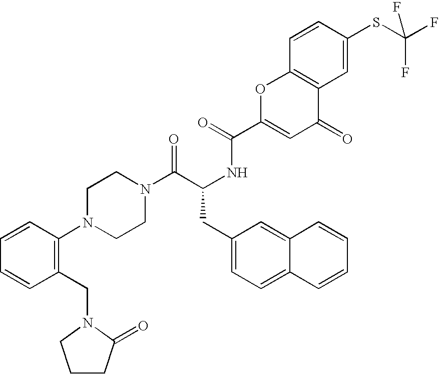

### EXAMPLE 289

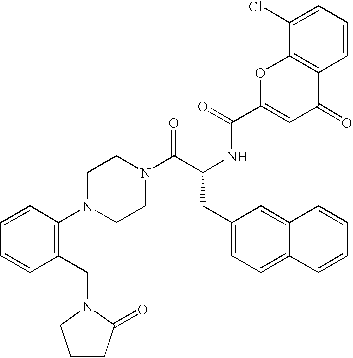

### EXAMPLE 290

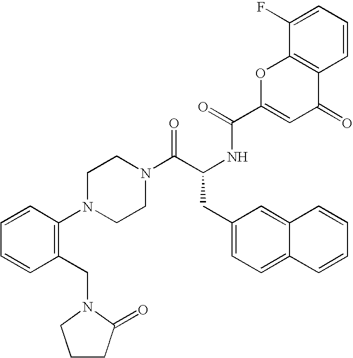

### EXAMPLE 291

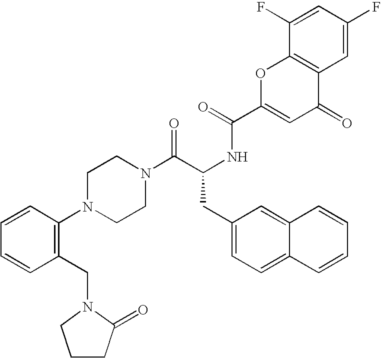

### EXAMPLE 292

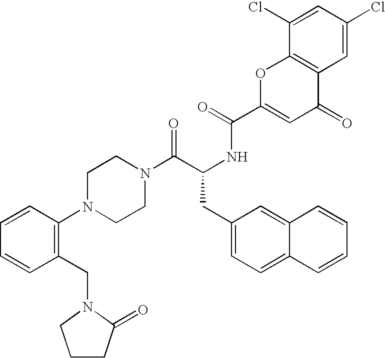

### EXAMPLE 293

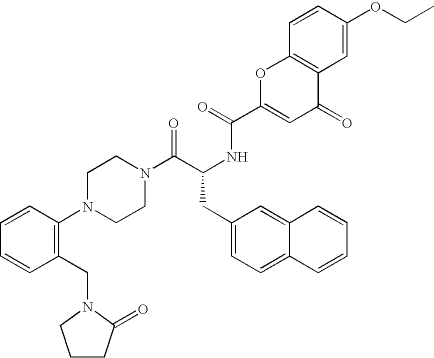

### EXAMPLE 294

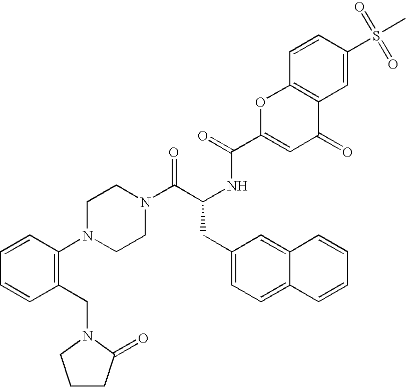

### EXAMPLE 295

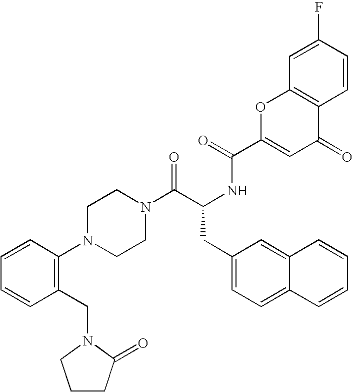

### EXAMPLE 296

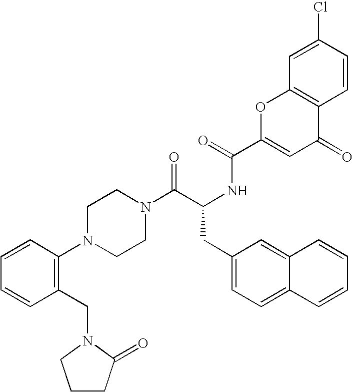

### EXAMPLE 297

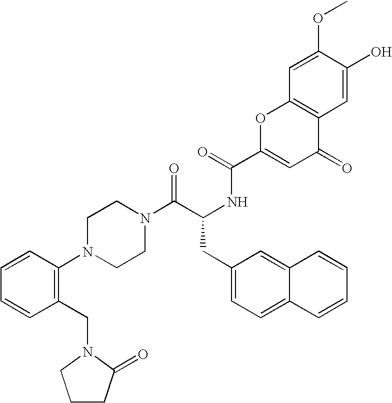

### EXAMPLE 298

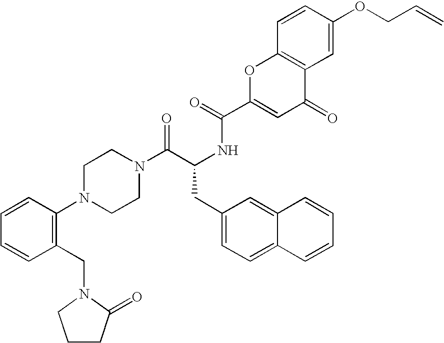

### EXAMPLE 299

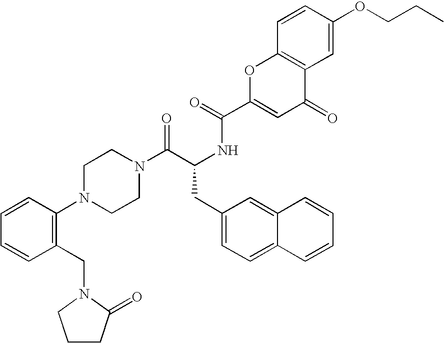

### EXAMPLE 300

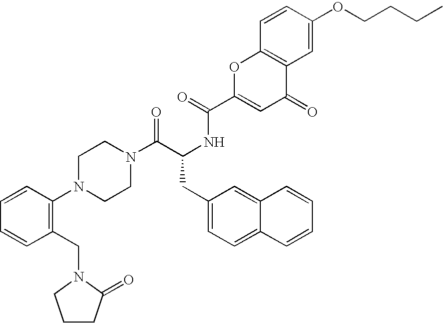

### EXAMPLE 301

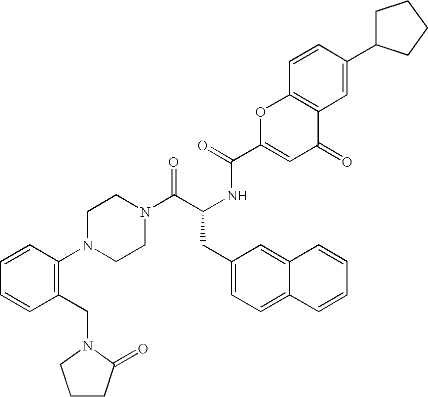

### EXAMPLE 302

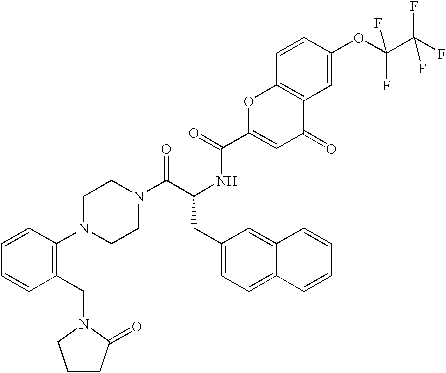

### EXAMPLE 303

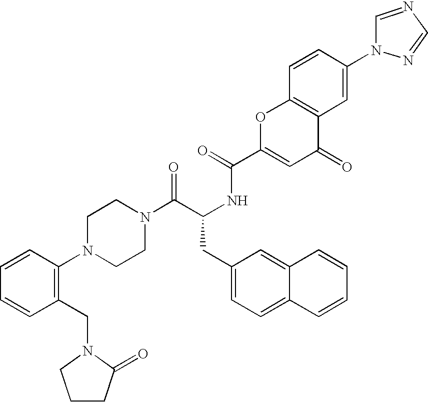

### EXAMPLE 304

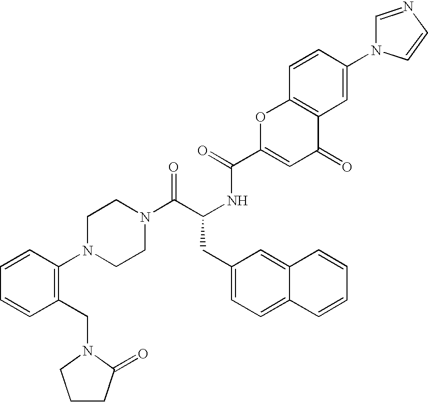

### EXAMPLE 305

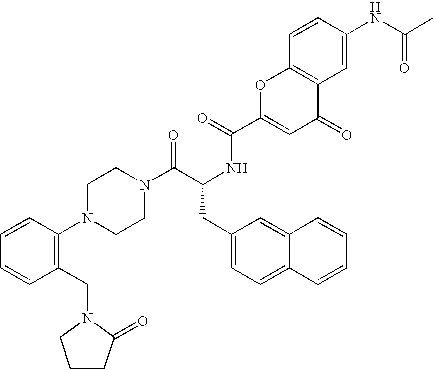

### EXAMPLE 306

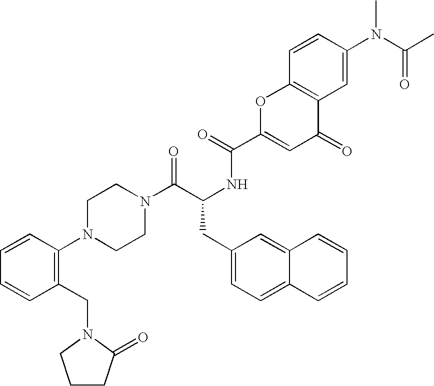

### EXAMPLE 307

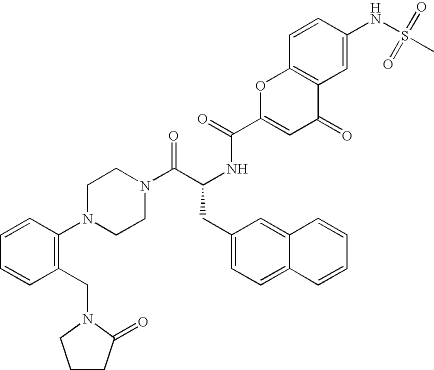

### EXAMPLE 308

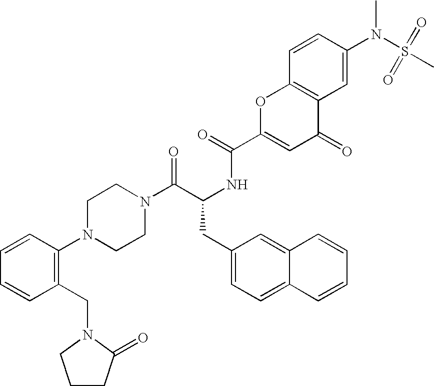

### EXAMPLE 309

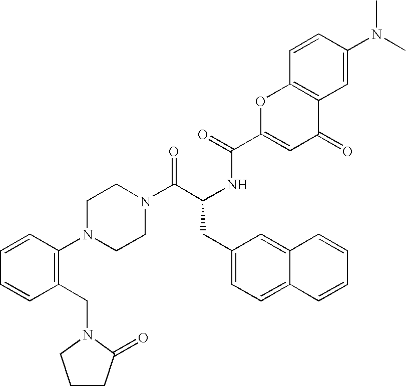

### EXAMPLE 310

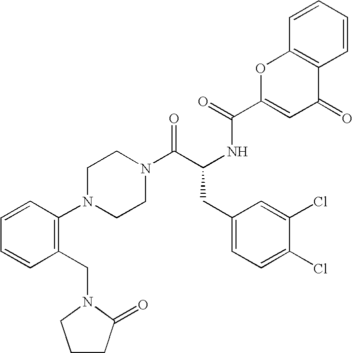

white solid

R_{f}=0.59 (ethyl acetate); Mp. 128-136° C.

### EXAMPLE 311

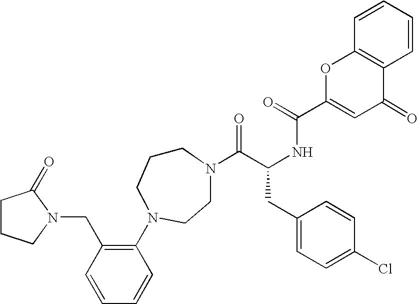

white solid

R_{f}=0.61 (ethyl acetate/ethanol 3:1); Mp. 108-122° C.

### EXAMPLE 312

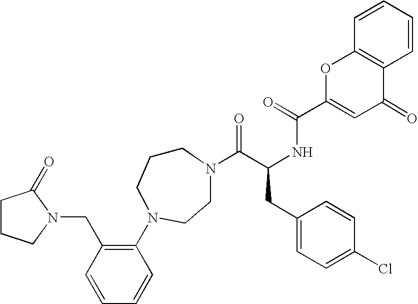

white solid

R_{f}=0.09 (ethyl acetate); Mp. 127-135° C.

### EXAMPLE 313

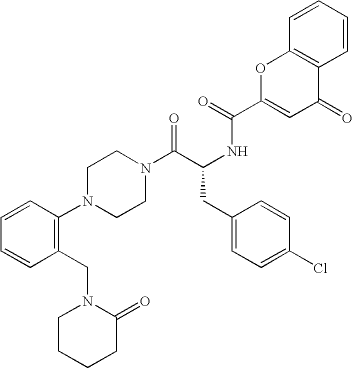

### EXAMPLE 314

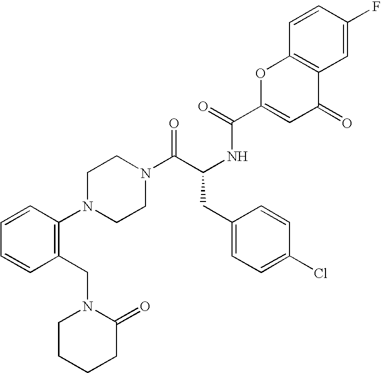

### EXAMPLE 315

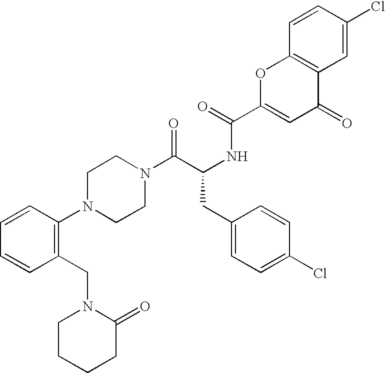

### EXAMPLE 316

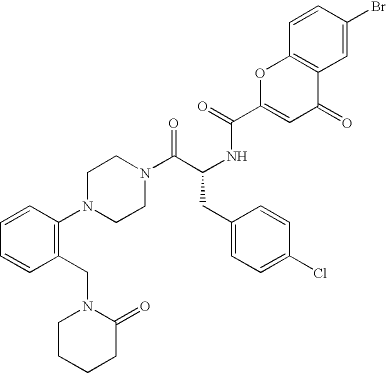

### EXAMPLE 317

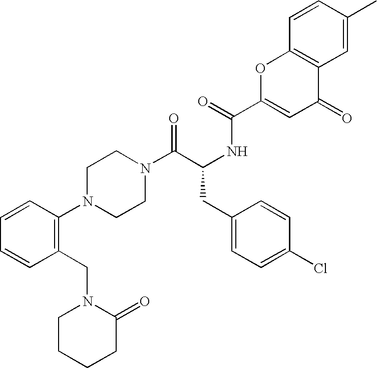

### EXAMPLE 318

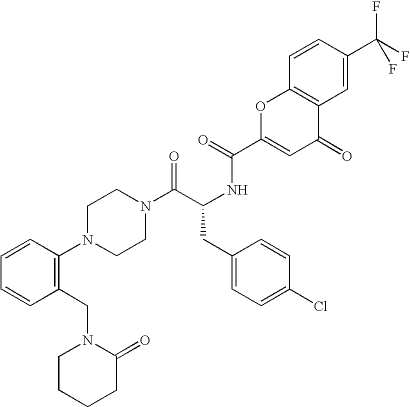

### EXAMPLE 319

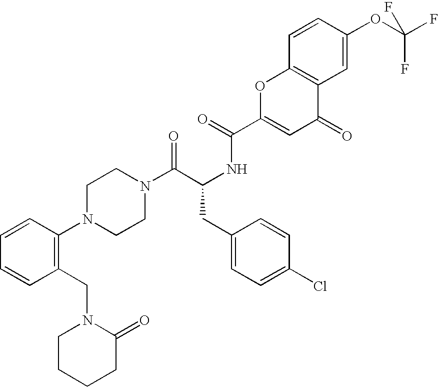

### EXAMPLE 320

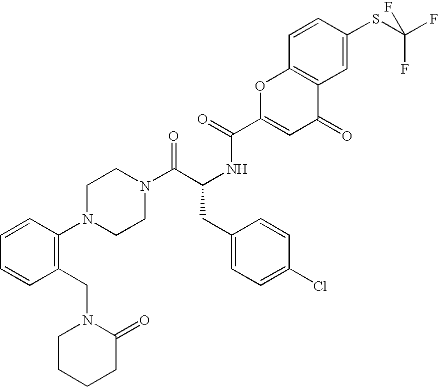

### EXAMPLE 321

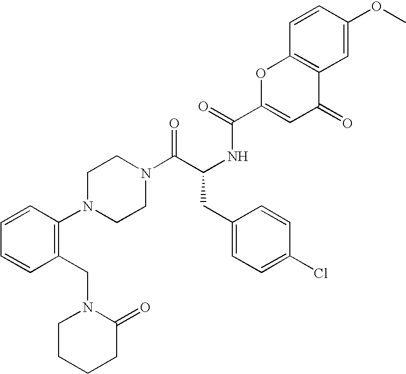

### EXAMPLE 322

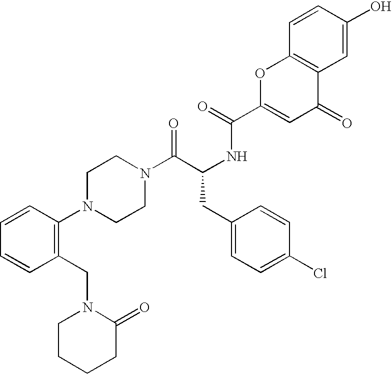

### EXAMPLE 323

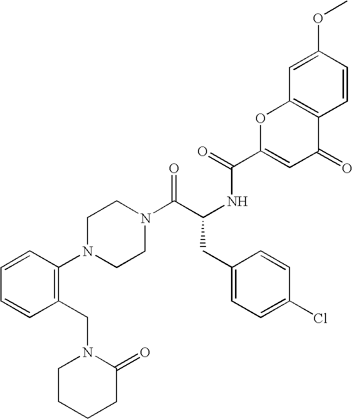

### EXAMPLE 324

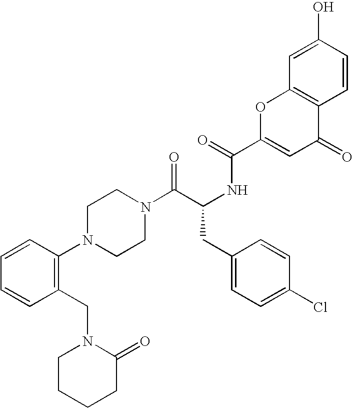

### EXAMPLE 325

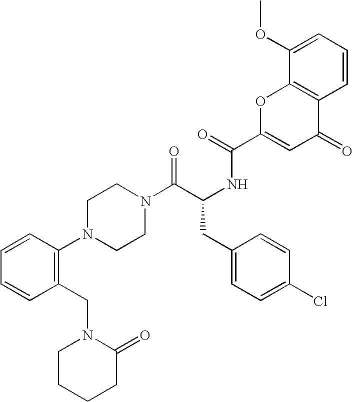

### EXAMPLE 326

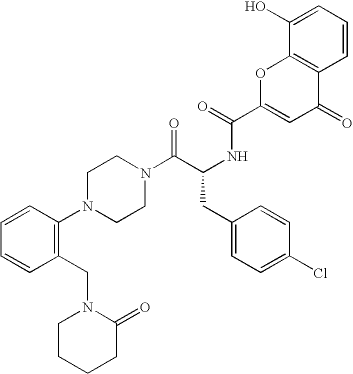

### EXAMPLE 327

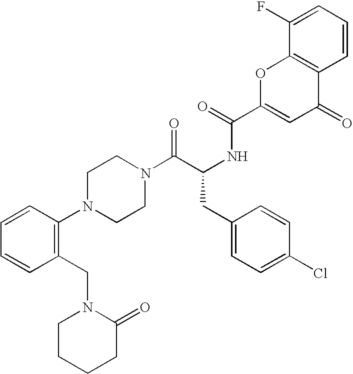

### EXAMPLE 328

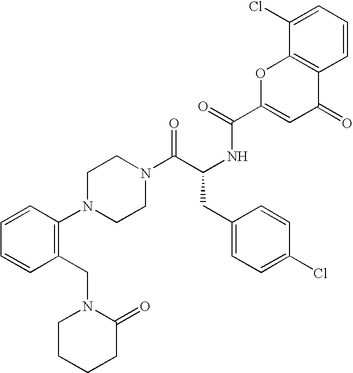

### EXAMPLE 329

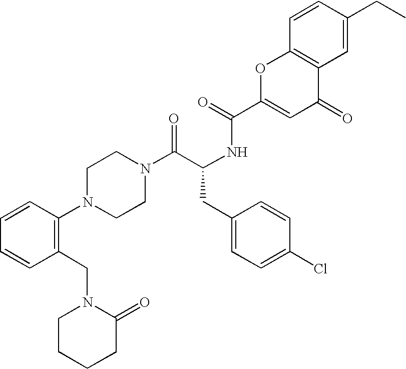

### EXAMPLE 330

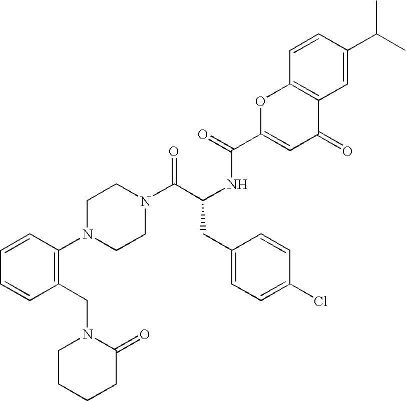

### EXAMPLE 331

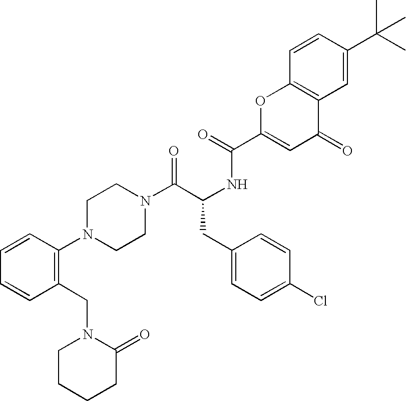

### EXAMPLE 332

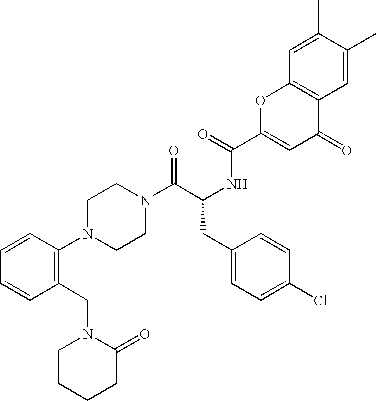

### EXAMPLE 333

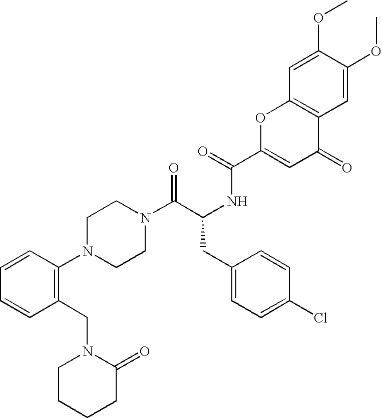

### EXAMPLE 334

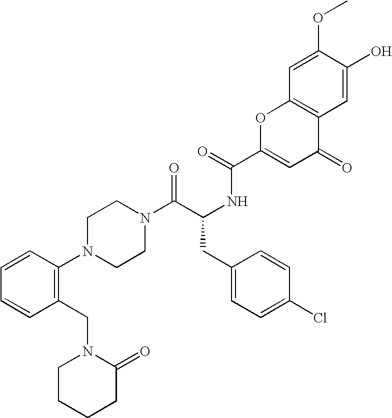

### EXAMPLE 335

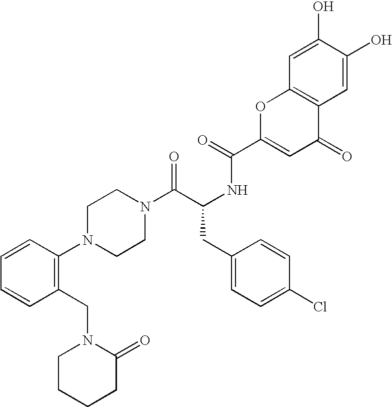

### EXAMPLE 336

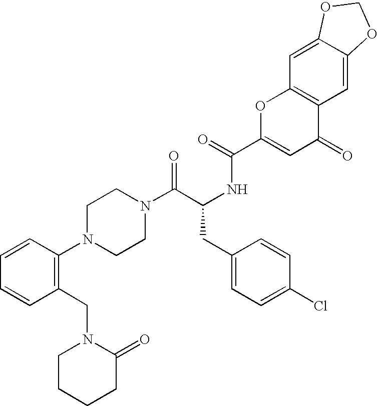

### EXAMPLE 337

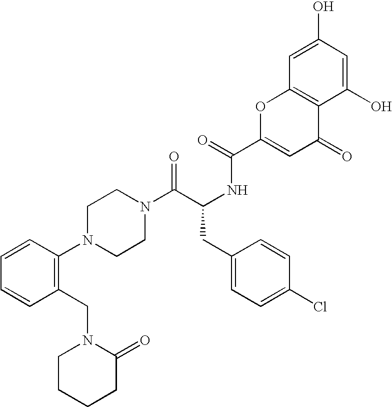

### EXAMPLE 338

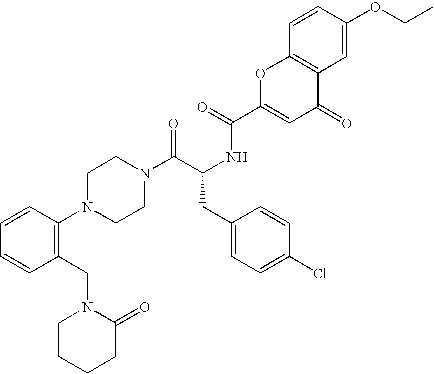

### EXAMPLE 339

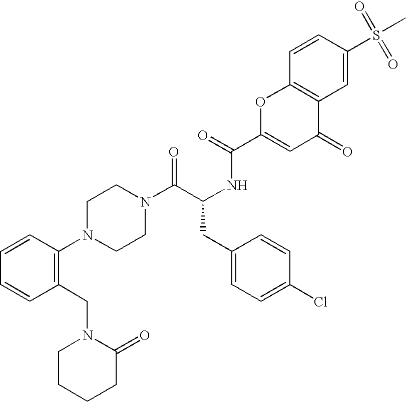

### EXAMPLE 340

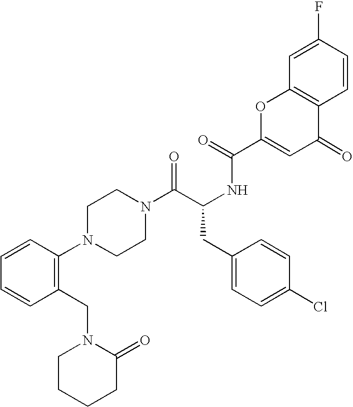

### EXAMPLE 341

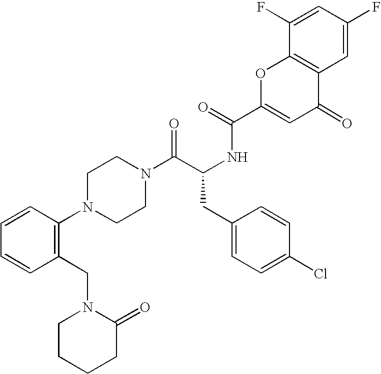

### EXAMPLE 342

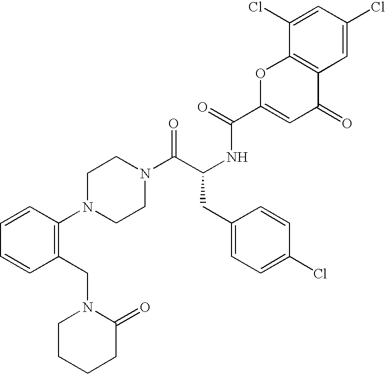

### EXAMPLE 343

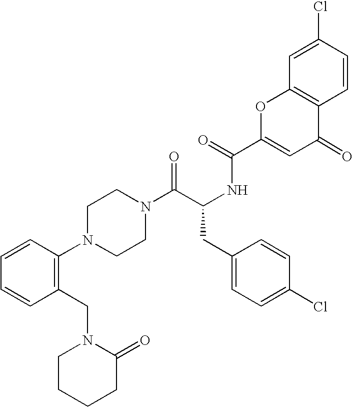

### EXAMPLE 344

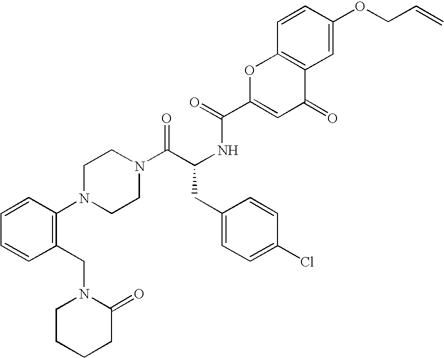

### EXAMPLE 345

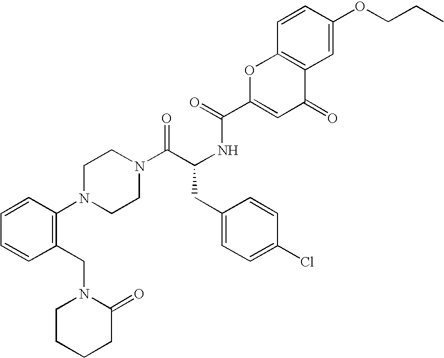

### EXAMPLE 346

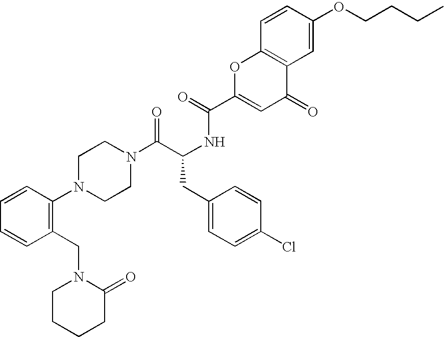

### EXAMPLE 347

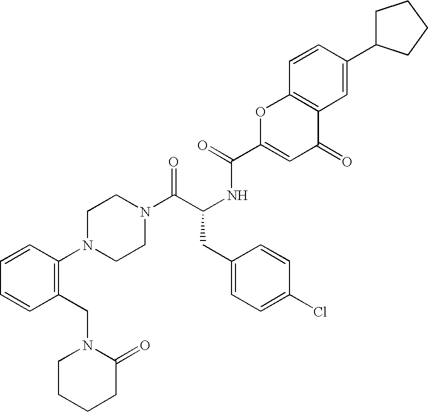

### EXAMPLE 348

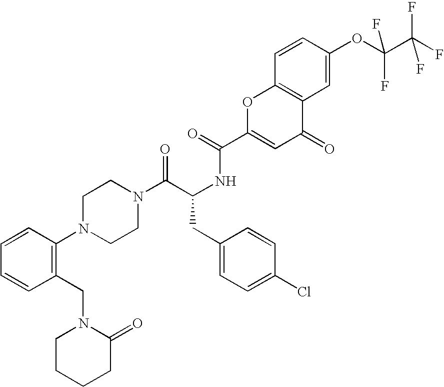

### EXAMPLE 349

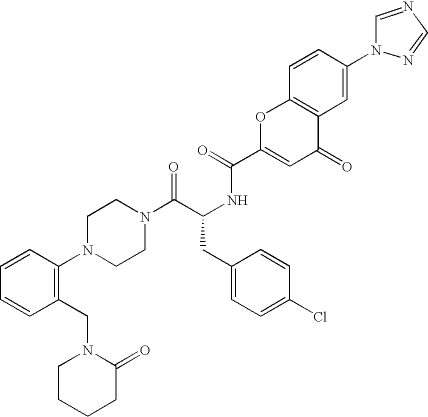

### EXAMPLE 350

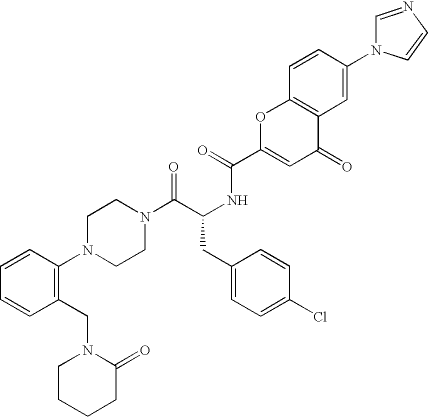

### EXAMPLE 351

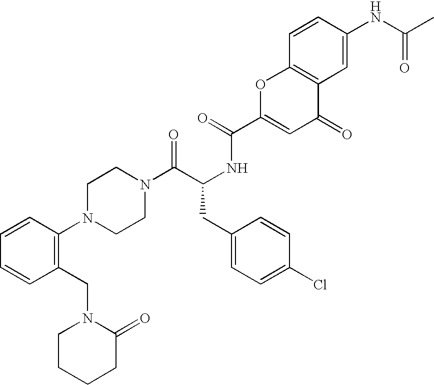

### EXAMPLE 352

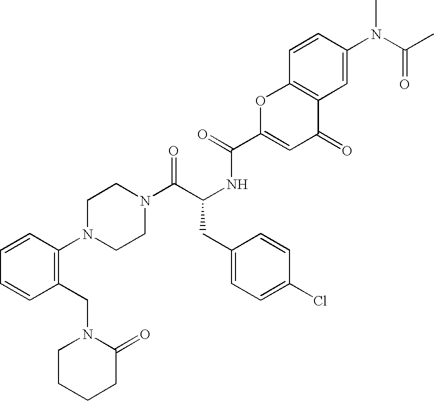

### EXAMPLE 353

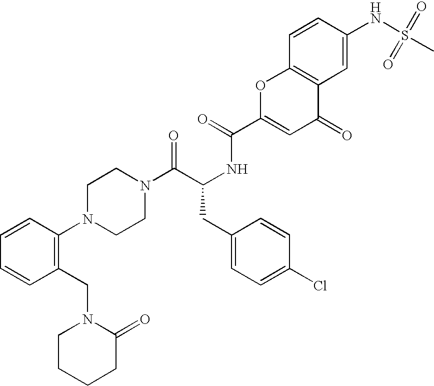

### EXAMPLE 354

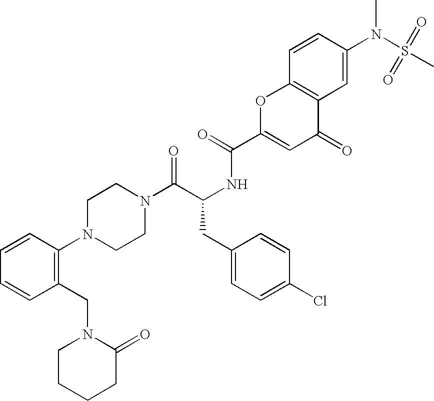

### EXAMPLE 355

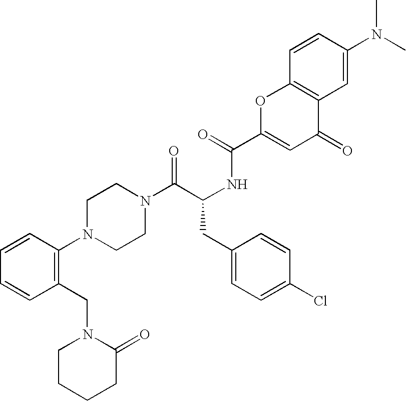 Preparation of the chromone-2-carboxylic acids:

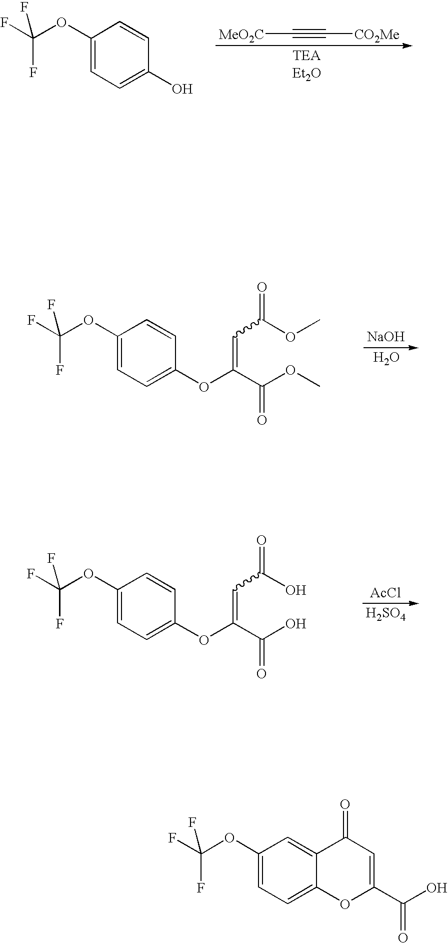

Synthesis of Chromone-2-carboxylic Acids using method 1 Chromone-2-carboxylic acid 1

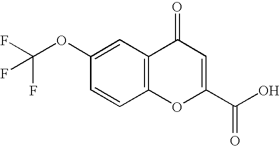

Intermediate CA1b) (5.85 g) was suspended in AcCl (110 ml) and concentrated sulfuric acid (4.40 ml) was added while stirring at RT. Then the slightly yellowish reaction mixture was heated to reflux with vigorous stirring and kept under reflux for 30 min. The reaction mixture was evaporated in vacuo to a volume of ca. 25 ml and then slowly and carefully added to well stirred H₂O (300 ml) and stirring was continued for 1 h. After brief sonication, the formed precipitate was filtered off, washed with cold H₂O (3×30 ml), and finally dried in vacuo at 40° C. overnight. The crude product was dissolved in a minimal amount of boiling H₂O (270 ml) and left to slowly cool to RT. Crystallization was completed at RT for 6 h, then the crystalline product was filtered off and washed with cold H₂O (3×10 ml). Finally the product was dried in vacuo at 40° C. overnight to yield the title compound.

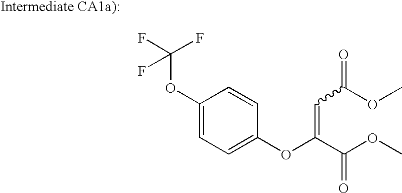

4-Trifluoromethoxyphenol (6.67 g) was dissolved in Et₂O (55 ml) and TEA (6.36 ml) was added while stirring at RT. Then dimethyl acetylendicarboxylate (5.12 ml) was added with vigorous stirring and the reaction mixture stirred at RT in the dark overnight. The reaction mixture was diluted with Et₂O (30 ml) and washed with 1 M HCl (3×65 ml), H₂O (30 ml), and brine (25 ml), dried with Na₂SO₄ and then evaporated in vacua. Finally it was dried under high vacuum for 2 h to yield the desired product.

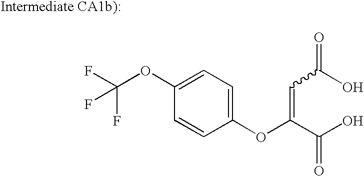

To intermediate CA1a) (9.57 g) was added a solution of NaOH (4.80 g) in water (45 ml) while stirring at RT. Then the reaction mixture was heated to reflux with vigorous stirring and kept under reflux for 3 h. The reaction mixture was extracted with Et₂O (100 ml) and then acidified to below pH 1 with conc. HCl while cooling in ice/H₂O. A white precipitate formed, which was filtered off, washed with H₂O (3×30 ml), and finally it was dried in vacuo at 40° C. overnight to give the desired compound.

The following chromone-2-carboxylic acids were prepared using method 1: 6-ethyl chromone-2-carboxylic acid, 6-isopropylchromone-2-carboxylic acid, 6-methoxychromone-2-carboxylic acid, 6-trifluoromethylchromone-2-carboxylic acid, 6-tert.-butylchromone-2-carboxylic acid, 6-chlorochromone-2-carboxylic acid, 6-trifluoromethoxychromone-2-carboxylic acid, 8-methoxychromone-2-carboxylic acid, 6-trifluoromethylsulfanylchromone-2-carboxylic acid, 8-chlorochromone-2-carboxylic acid, 8-fluorochromone-2-carboxylic acid 7-chlorochromone-2-carboxylic acid, 6-ethoxychromone-2-carboxylic acid, 6-methanesulfonylchromone-2-carboxylic acid, 8-oxo-8H-[1,3]dioxolo[4,5-g]chromene-6-carboxylic acid, 6-allyloxy-4-hydroxy-4H-chromene-2-carboxylic acid, 6-butoxy-4-hydroxy-4H-chromene-2-carboxylic acid, 6-propoxy-4-hydroxy-4H-chromene-2-carboxylic acid, 6-cyclopentyl-4-oxo-4H-chromene-2-carboxylic acid, 6-pentafluoroethoxy-4-oxo-4H-chromene-2-carboxylic acid, 4-oxo-6-[1,2,4]triazol-1-yl-4H-chromene-2-carboxylic acid, 6-imidazol-1′-yl-4-oxo-4H-chromene-2-carboxylic acid, 6-acetylamino-4-oxo-4H-chromene-2-carboxylic acid, 6-(acetyl-methyl-amino)-4-oxo-4H-chromene-2-carboxylic acid, 6-methanesulfonylamino-4-oxo-4H-chromene-2-carboxylic acid, 6-(methanesulfonyl-methyl-amino)-4-oxo-4H-chromene-2-carboxylic acid and 6-dimethylamino-4-oxo-4H-chromene-2-carboxylic acid.

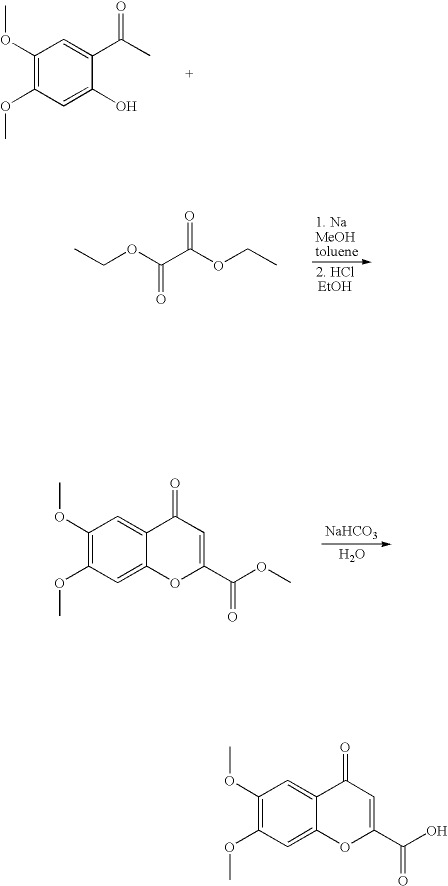

Synthesis of Chromone-2-carboxylic Acids using method 2 Chromone-2-carboxylic acid 2

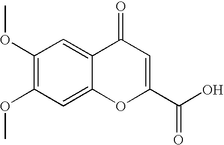

Intermediate CA2a) (2.65 g) was suspended in sat. sodium bicarbonate solution (50 ml) and heated to 80° C. for 2 h. At the end of the reaction a clear solution was obtained. After cooling to room temperature the reaction mixture was acidified with HCl. The white precipitate was filtered off, washed with water and dried in vacuo at 40° C. overnight to give the title compound.

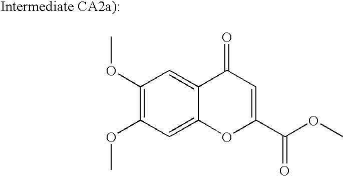

Sodium (4.0 g) was added to dry methanol (50 ml). After the conversion to the methoxide was complete the solution was cooled and a solution of 2′-hydroxy-4,5′-dimethoxyacetophenone (3.92 g) in diethyl oxalate (12 ml), methanol (50 ml) and toluene (50 ml) was added to it. The mixture was refluxed overnight. After cooling, diethyl ether (200 ml) was added. The sodium salt was filtered, washed with anhydrous ether, suspended in water and the solution acidified. The resultant precipitate was filtered and dried to yield a yellow solid.

The intermediate was dissolved in ethanol (100 ml) and heated at 100° C. for 15 min; concentrated HCl (2 ml) was added, and the solution stirred at 100° C. for 1.5 h. Immediately after addition of the acid a precipitate was formed. After cooling to room temperature the reaction mixture was diluted with water (150 ml) and the pale yellow precipitate was filtered off and washed with water. The product was dried under reduced pressure.

The following chromone-2-carboxylic acids were prepared using method 2: 6-methoxychromone-2-carboxylic acid, 7-methoxychromone-2-carboxylic acid, 6,7-dimethylchromone-2-carboxylic acid, 6,7-dimethoxychromone-2-carboxylic acid, 6-chlorochromone-2-carboxylic acid, 6,8-difluorochromone-2-carboxylic acid, 6,8-dichlorochromone-2-carboxylic acid and 7-fluorochromone-2-carboxylic acid.

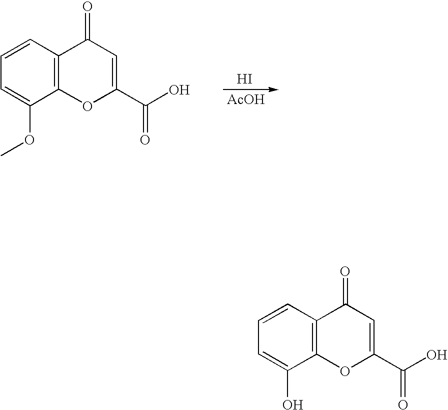

Demethylation of Methoxy Substituted Chromone-2-carboxylic Acids Chromone-2-carboxylic acid 3

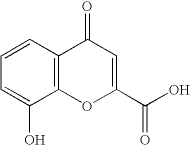

8-Methoxychromone-2-carboxylic acid (220 mg) was suspended in AcOH (2 ml) and conc. HI (2 ml) was added while stirring at RT. Then the slightly yellowish suspension was heated to 120° C. with vigorous stirring and kept at this temperature for 60 min. The warm reaction mixture was slowly and carefully added to well stirred H₂O (75 ml) and the resulting yellow solution was chilled in ice for 30 min. Crystallization was completed in the fridge for another 2 h. The formed crystalline precipitate was filtered off, washed with cold H₂O (3×3 ml), and finally dried in vacuo at 40° C. overnight.

The following chromone-2-carboxylic acids were prepared using the demethylation method:

6-hydroxychromone-2-carboxylic acid, 7-hydroxychromone-2-carboxylic acid, 8-hydroxychromone-2-carboxylic acid, 6,7-dihydroxychromone-2-carboxylic acid and 6-hydroxy-7-methoxychromone-2-carboxylic acid.

Biological Assays

A. Binding Assay

A membrane binding assay is used to identify competitive inhibitors of fluorescence labeled NDP-alpha-MSH binding to HEK293 cell membrane preparations expressing human melanocortin receptors.

The test compound or unlabeled NDP-alpha-MSH is dispensed at varying concentrations to a 384 well microtiter plate. Fluorescence labeled NDP-alpha-MSH is dispensed at a single concentration, followed by addition of membrane preparations. The plate is incubated for 5 h at room temperature.

The degree of fluorescence polarization is determined with a fluorescence polarization microplate reader.

B. Functional Assay

A functional cellular assay, based on competition between unlabeled CAMP and a fixed quantity of fluorescence labeled CAMP for a limited number of binding sites on a CAMP specific antibody, is used to discriminate melanocortin receptor agonists from antagonists by fluorescence polarization.

HEK293 cells expressing one of the human melanocortin receptors are transferred to 384 well microtiter plates, an appropriate amount of CAMP antibody is added, followed by the addition of different concentrations of the test compound to effect cAMP production. Cells are lysed and a fluorescence labeled cAMP conjugate is dispensed. The plate is read on a fluorescence polarization microplate reader and the amount of cAMP produced as a response to a test compound is compared to the production of CAMP resulting from stimulation with NDP-alpha-MSH.

To define antagonistic activity of a test compound, the compound is dispensed at different concentrations to cells stimulated by an appropriate amount of NDP-α-MSH. Inhibition of CAMP production is determined by comparing the inhibition of CAMP production of the test compound to the inhibition of cAMP production by a known inhibitor tested at the same concentrations.

Biological Data for selected Examples of the Invention:

| | hMC4-R | hMC4-R | |
|---|---|---|---|
| | binding assay | functional | % activation |
| Example | IC₅₀/nM | assay EC₅₀/μM | functional assay |
| 1 | 200 | — | no activation |
| 2 | 36 | 5.7 | 43 |
| 91 | 120 | — | no activation |
| 92 | 200 | — | no activation |
| 93 | 51 | — | no activation |
| 178 | 200 | — | no activation |
| 179 | 500 | — | no activation |
| 180 | 47 | — | no activation |
| 181 | 770 | — | no activation |
| 182 | 61 | — | 15%@10 μM |
| 267 | 100 | — | no activation |

C. In Vivo Food Intake Models
1. Spontaneous Feeding Paradigm

Food intake in rats is measured after i.p. or p.o. administration of the test compound (see e.g. Chen, A. S. et al. Transgenic Res 2000 April 9(2):145-54).

Selected Examples of the present invention were active in the rat model at 3, 10 or 30 mg/kg after i.p. and p.o. administration of the test compound, respectively, using male Wistar rats (n=4).

Example 1 at 30 mg/kg p.o. administration lead to an increase in cumulative food intake of 159% (4 hours following administration p=0.045, n=4), 131% (6 hours following administration, p=0.059, n=4) and 148% (7 hours following administration, p=0.037, n=4), respectively, compared to control male Wistar rats receiving vehicle only (n=8).

Example 17 at 10 mg/kg p.o. administration lead to an increase in cumulative food intake of 3100% (2 hours following administration p=0.029, n=4) and 540% (4 hours following administration p=0.035, n=4, respectively, compared to control male Wistar rats receiving vehicle only (n=4).

Example 182 at 3 mg/kg p.o. administration lead to an increase in cumulative food intake of 273% (2 hours following administration p=0.030, n=4), 204% (4 hours following administration p=0.040, n=4), 156% (6 hours following administration, p=0.050, n=4) and 197% (7 hours following administration, p=0.010, n=4), respectively, compared to control male Wistar rats receiving vehicle only (n=8).

2. Model of LPS- and Tumor-Induced Cachexia

Prevention or amelioration of cachexia induced by either lipopolysaccharide (LPS) administration or by tumor growth is determined upon i.p. or p.o. administration of test compounds to rats (see e.g. Marks, D. L.; Ling, N, and Cone, R. D. Cancer Res 2001 Feb. 15;61 (4): 1432-8)

a) Lipopolysaccharide-Induced Cachexia in Rats (Acute Model)

1-2 Hours prior to the onset of the dark-phase, individually housed, male Wistar rats (200-300 g) receive an ip or po application of test-compound or vehicle (2 ml/kg, 1-30 mg/kg) which is followed or preceded by an ip injection of either lipopolysaccharide (LPS) or saline (2 ml/kg, 100 μg/kg). Food intake, water intake and body weight are measured at 1-24 hour intervals and differences between experimental groups are evaluated.

b) Tumour-Induced Cachexia in Mice (Chronic Model)

Subcutaneous injection of Lewis lung carcinoma cells to male C57BL6 mice (1 million cells/100 μl/mouse) results in non-metastasizing tumor growth which in turn results in loss of lean body mass. Chronic ip or po applications of test compounds (10 ml/kg, 1-30 mg/kg for 7-21 days) are accompanied by daily measurements of food intake, water intake and body weight. Lean body mass is measured at the start, during and at the termination of the study using magnetic resonance relaxometry, and at the end of the study using a conventional chemical extraction procedure (Soxhlet's extraction). Differences between experimental groups are evaluated.

D. Rat Ex Copula Assay

Sexually mature male Caesarian Derived Sprague Dawley (CD) rats (over 60 days old) are used with the suspensory ligament surgically removed to prevent retraction of the penis, back into the penile sheath during the ex copula evaluations. Animals receive food and water ad lib and are kept on a normal light/dark cycle. Studies are conducted during the light cycle.

1. Conditioning to Supine Restraint for Ex Copula Reflex Tests

This conditioning takes about 4 days. Day 1, the animals are placed in a darkened restrainer and left for 15-30 minutes. Day 2, the animals are restrained in a supine position in the restrainer for 15-30 minutes. Day 3, the animals are restrained in the supine position with the penile sheath retracted for 15-30 minutes. Day 4, the animals are restrained in the supine position with the penile sheath retracted until penile responses are observed. Some animals require additional days of conditioning before they are completely acclimated to the procedures; non-responders are removed from further evaluation. After any handling or evaluation animals are given a treat to ensure positive reinforcement.

2. Ex Copula Reflex Tests

Rats are gently restrained in a supine position with their anterior torso placed inside a cylinderof adequate size to allow for normal head and paw grooming. For a 400-500 gram rat, the diameter of the cylinder is approximately 8 cm. The lower torso and hind limbs are restrained with a nonadhesive material (vetrap). An additional piece of vetrap with a hole in it, through which the glans penis will be passed, is fastened over the animal to maintain the preputial sheath in a retracted position. Penile responses will be observed, typically termed ex copula genital reflex tests. Typically, a series of penile erections will occur spontaneously within a few minutes after sheath retraction. The types of normal reflexogenic erectile responses include elongation, engorgement, cup and flip. An elongation is classified as an extension of the penile body. Engorgement is a dilation of the glans penis. A cup is defined as an intense erection where the distal margin of the glans penis momentarily flares open to form a cup. A flip is a dorsiflexion of the penile body.

Baseline and or vehicle evaluations are conducted to determine how and if an animal will respond. Some animals have a long duration until the first response while others are non-responders altogether. During this baseline evaluation latency to first response, number and type of responses are recorded. The testing time frame is 15 minutes after the first response.

After a minimum of 1 day between evaluations, these same animals are administered the test compound at 20 mg/kg and evaluated for penile reflexes. All evaluations are videotaped and scored later. Data are collected and analyzed using paired, 2 tailed t-tests, to compared baseline and/or vehicle evaluations to drug treated evaluations for individual animals. Groups of a minimum of 4 animals are utilized to reduce variability.

Positive reference controls are included in each study to assure the validity of the study. Animals can be dosed by a number of routes of administration depending on the nature of the study to be performed. The routes of administration includes intravenous (IV), intraperitoneal (IP), subcutaneous (SC) and intracerebral ventricular (ICV).

E. Models of Female Sexual Dysfunction

Rodent assays, relevant to female sexual receptivity, include the behavioral model of lordosis and direct observations of copulatory activity. There is also an urethrogenital reflex model in anesthetized spinally transected rats for measuring orgasm in both male and female rats. These and other established animal models of female sexual dysfunction are described in McKenna K E et al, A Model For The Study of Sexual Function In Anesthetized Male And Female Rats, Am. J. Physiol. (Regulatory Integrative Comp. Physiol 30): R1276-R1285, 1991; McKenna K E et al, Modulation By Peripheral Serotonin of The Threshold For Sexual Reflexes In Female Rats, Pharm. Bioch. Behav., 40:151-156, 1991; and Takahashi L K et al, Dual Estradiol Action In The Diencephalon And The Regulation of Sociosexual Behavior In Female Golden Hamsters, Brain Res., 359:194-207,1985.

As evident from the results presented above, representative compounds of the present invention bind to the human melanocortin-4 receptor. Representative compounds of the present invention were also tested in the functional assay and found to be non-activating or very weakly activating the melanocortin-4 receptor with high EC₅₀ values and low stimulation.

Compounds bearing an o-substituted arylpiperazine “A moiety” in combination with D-p-chlorophenylalanine as “B moiety” and D-Tic as “C moiety” are known to bind to the melanocortin-4 receptor with Kᵢ values between 24 nM and 6.6 μM (J. Med. Chem. 2004, 47, 744-755, 29 examples) and to activate melanocortin-4 receptor in the functional assay with EC₅₀ values between 14 nM and 1.3 μM (J. Med. Chem. 2004, 47, 744-755, 29 examples) and between 4 nM and 4.4 μM (Bioorg. Med. Chem. Lett. 2003, 13, 3793-3796, 23 examples). In the latter case three additional examples are reported to be weak agonists with 7- to 30-fold stimulation at 30 μM. There is no compound described which does not activate the melanocortin-4 receptor. One of the compounds described above is claimed in patent application WO03009850 (example 1). The compound is described to bind to the melanocortin-4 receptor with Kᵢ=220 nM and to activate said receptor with an EC₅₀=16 nM (J. Med. Chem. 2004, 47, 744-755, compound 39). Another source describes said compound to activate the melanocortin-4 receptor with an EC₅₀=380 nM (100% stimulation) (Bioorg. Med. Chem. Lett. 2003, 13, 3793-3796, compound 3). In our assays the compound was found to have a melanocortin-4 receptor binding IC₅₀=500 nM and to activate the melanocortin-4 receptor with an EC₅₀=3.7 μM (96% activation).

There is evidence in the literature that the stereochemistry of Tic in the “C moiety” does not have a big influence on the melanocortin-4 receptor affinity and activation at a concentration of 10 μM (J. Med. Chem. 2002, 45, 4589-4593, compounds 1 and 13). Both diastereomers activate the melanocortin-4 receptor, however, the D-Tic derived compound 1 shows improved functional activity. Applying this concept to compounds bearing the o-substituted arylpiperazine “A moiety” it can be concluded that the diastereomers of the compounds described in the literature cited above also act as agonists. Some of the L-Tic derivatives are described in WO03009850 (examples 56, 66, 91, 93, 117, 118 and 423).

In a recently published paper a series of compounds is described where the replacement of D-Tic with β-alanine derivatives leads to compounds with potent affinity for the melanocortin-4 receptor and intrinsic activities of >90% (Bioorg. Med. Chem. Left. 2003, 13, 4341-4344,). Some of the β-alanine derivatives were also used as “C moiety” in WO03009850, e.g. azetidine-3-carboxylic acid and piperidine-3-carboxylic acid.

Example 1 of the present invention binds to the melanocortin-4 receptor with an IC₅₀=200 nM and does not activate the melanocortin-4 receptor in the functional assay. Therefore, in contrast to all the compounds discussed above, example 1 is an antagonist.

The enantiomer of example 1, example 2, binds to the melanocortin-4 receptor with an IC₅₀=36 nM and only weakJy activates the receptor (43% activation) at a high concentration, therefore being an antagonist. The corresponding compound with azetidine-3-carboxylic acid as “C moiety” (WO003009847, example 153) binds to the melanocortin-4 receptor with an IC₅₀=40 nM and activates said receptor with an EC₅₀=1.0 μM (107% activation), therefore being a full agonist.

Example 91 of the present invention binds to the melanocortin-4 receptor with an IC₅₀=120 nM and does not activate the melanocortin-4 receptor in the functional assay. Therefore example 91 is an antagonist.

Example 92 of the present invention binds to the melanocortin-4 receptor with an IC₅₀=200 nM and does not activate the melanocortin-4 receptor in the functional assay. Therefore example 92 is an antagonist.

Example 93 of the present invention binds to the melanocortin-4 receptor with an IC₅₀=51 nM and does not activate the melanocortin-4 receptor in the functional assay. Therefore example 93 is an antagonist.

Example 178 of the present invention binds to the melanocortin-4 receptor with an IC₅₀=200 nM and does not activate the melanocortin-4 receptor in the functional assay. Therefore example 178 is an antagonist.

Example 179 of the present invention binds to the melanocortin-4 receptor with an IC50=500 nM and does not activate the melanocortin-4 receptor in the functional assay. Therefore example 179 is an antagonist.

Example 180 of the present invention binds to the melanocortin-4 receptor with an IC₅₀=47 nM and does not activate the melanocortin-4 receptor in the functional assay. Therefore example 180 is an antagonist.

Example 181 of the present invention binds to the melanocortin-4 receptor with an IC₅₀=770 nM and does not activate the melanocortin-4 receptor in the functional assay. Therefore example 181 is an antagonist.

The enantiomer of example 181, example 182, binds to the melanocortin-4 receptor with an IC₅₀=61 nM and induces little or no activation at 10 μM. The corresponding compound with azetidine-3-carboxylic acid as “C moiety” (WO03009850, examples 166) binds to the melanocortin-4 receptor with an IC₅₀=110 nM and activates said receptor with an EC₅₀=2.9 μM (90% activation). Therefore example 182 is an antagonist whereas the prior art compound with azetidine-3-carboxylic acid is an agonist.

Example 267 of the present invention binds to the melanocortin-4 receptor with an IC₅₀=100 nM and does not activate the melanocortin-4 receptor in the functional assay. Therefore example 267 is an antagonist.

As illustrated by the biological results (see above) representative compounds of the present invention are also active as antagonists when tested in vivo.

Examples 1, 17 and 182 are active in the spontaneous feeding paradigm. The test animals show a significant increase in food intake at dose of 3 to 30 mg/kg p.o.

Examples of a Pharmaceutical Composition

As a specific embodiment of an oral composition of a compound of the present invention, 20 mg of Example 17 is formulated with sufficient finely divided lactose to provide a total amount of 580 to 590 mg to fill a size 0 hard gelatin capsule.

As another specific embodiment of an oral composition of a compound of the present invention, 15 mg of Example 182 is formulated with sufficient finely divided lactose to provide a total amount of 580 to 590 mg to fill a size 0 hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth above may be applicable as a consequence of the specific pharmacological responses observed and may vary depending upon the particular active compound selected, as well as from the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound of structural formula (I): 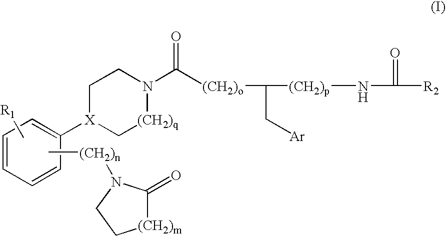
or a pharmaceutically acceptable salt or solvate thereof, wherein
Ar is:
aryl or heteroaryl which may both be substituted or unsubstituted;
R₁ is independently:
hydrogen,
hydroxy,
cyano,
nitro,
halo,
alkyl,
alkoxy or
haloalkyl;
R₂ is: 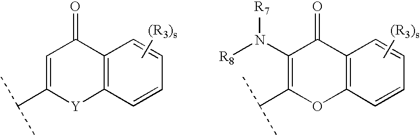
each R₃ is independently:
hydrogen,
halo,
alkyl,
haloalkyl,
hydroxy,
alkoxy,
S-alkyl,
SO₂-alkyl,
O-alkenyl,
S-alkenyl,
NR₇C(O)R₇,
NR₇SO₂R₇,
N(R₇)₂
(D)-cycloalkyl,
(D)-aryl,
(D)-heteroaryl or
(D)-heterocyclyl (wherein heterocyclyl excludes a heterocyclyl containing a single nitrogen), and
wherein aryl, heteroaryl, heterocyclyl, alkyl and/or cycloalkyl may be substituted or unsubstituted, and two adjacent R₃ may form a 4- to 7-membered ring;
R₇ and R₈ are each independently:
hydrogen,
alkyl or
cycloalkyl, or
R₇ and R₈ together with the nitrogen to which they are attached form a 5- to 8-membered ring,
wherein alkyl and cycloalkyl are both unsubstituted or substituted;
D is a bond or alkyl;
X is CH or N;
Y is O or NR₇;
n is 1-4;
m is 0-3;
o is 0-2;
p is 0-2;
q is 1 or 2;
s is 0-4.

2. The compound of claim 1, wherein
Ar is:
aryl which may be substituted with one to three substituents independently selected from the group consisting of cyano, nitro, perfluoroalkoxy, halo, alkyl, (D)-cycloalkyl, alkoxy and/or haloalkyl;
R₁ is independently:
hydrogen,
hydroxy,
halo,
alkyl,
alkoxy or
haloalkyl;
R₂ is: 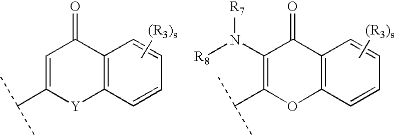
each R₃ is independently:
hydrogen,
halo,
alkyl,
haloalkyl,
hydroxy,
alkoxy,
S-alkyl or
SO₂-alkyl,
O-alkenyl or
S-alkenyl;
R₇ and R₈ are each independently:
hydrogen,
alkyl or
cycloalkyl, or
R₇ and R₈ together with the nitrogen to which they are attached form a 5- to 7-membered ring optionally containing an additional heteroatom selected from O, S and NR₄;
D is a bond or CH₂;
X is CH or N;
Y is NR₇ or O;
n is 1 or 2;
m is 1-3;
o is 0 or 1;
p is 0 or 1;
q is 1;
s is 1-3.

3. The compound of claim 1, wherein
Ar is:
phenyl or naphthyl which may be substituted with one or two substituents independently selected from the group consisting of perfluoroalkoxy, halo, alkyl, alkoxy and haloalkyl;
R₁ is independently:
hydrogen,
alkoxy,
halo or
alkyl;
R₂ is: 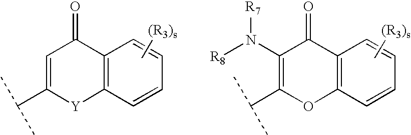
each R₃ is independently:
hydrogen,
hydroxy,
alkoxy,
SO₂-alkyl or
iso-propyl;
R₇ and R₈ are each independently:
hydrogen or
alkyl, or
R₇ and R₈ together with the nitrogen to which they are attached form a 6-membered ring optionally containing an additional oxygen atom;
X is CH or N;
Y is N-alkyl or O;
n is 1;
m is 1-3;
o is 0 or 1;
p is 0 or 1;
q is 1.

4. The compound of claim 1, wherein
Ar is:
phenyl or naphthyl which may be substituted with halo;
R₁ is hydrogen;
R₂ is: 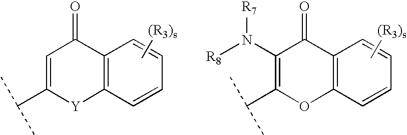
each R₃ is independently:
hydrogen,
hydroxy,
alkoxy,
SO₂-alkyl or
iso-propyl;
R₇ and R₈ are each independently:
hydrogen or
alkyl, or
R₇ and R₈ together with the nitrogen to which they are attached form a 5- to 6-membered ring optionally containing an additional oxygen atom;
X is CH or N;
n is 1;
m is 1 or 2;
o is 0;
p is 0;
q is 1
s is 1-2.

5. A medicament comprising the compound of claim 1.

6. A method of treating or preventing disorders, diseases or conditions responsive to the modulation of the melanocortin-4 receptor in a mammal, where modulation means activation in the case of MC4-R agonists or inactivation in the case of MC4-R antagonists, the method comprising administering to a human or mammal an effective amount of the compound of claim 1.

7. A method of treating or preventing cancer cachexia, the method comprising administering to a human or mammal an effective amount of the MC4-R antagonists according to claim 6.

8. A method of treating or preventing muscle wasting the method comprising administering to a human or mammal an effective amount of the MC4-R antagonists according to claim 6.

9. A method of treating or preventing anorexia, the method comprising administering to a human or mammal an effective amount of the MC4-R antagonists according to claim 6.

10. A method of treating or preventing anxiety and/or depression, the method comprising administering to a human or mammal an effective amount of the MC4-R antagonists according to claim 6.

11. A method of treating or preventing obesity the method comprising administering to a human or mammal an effective amount of the MC4-R antagonists according to claim 6.

12. A method of treating or preventing diabetes mellitus, the method comprising administering to a human or mammal an effective amount of the MC4-R antagonists according to claim 6.

13. A method of treating or preventing male or female sexual dysfunction the method comprising administering to a human or mammal an effective amount of the MC4-R antagonists according to claim 6.

14. A method of treating or preventing erectile dysfunction, the method comprising administering to a human or mammal an effective amount of the MC4-R antagonists according to claim 6.

15. A pharmaceutical composition which comprises a compound of claim 1 and a pharmaceutically acceptable carrier.
